(19) United States
(12) Patent Application Publication
(10) Pub. No.: US 2003/0087944 A1
(43) Pub. Date: May 8, 2003

(54) Method for the treatment of renal dysfunction with spla2 inhibitors

(76) Inventors: William Louis Macias, Indianapolis, IN (US); Vincent Phillip Meador, Greenfield, IN (US)

Correspondence Address: ELI LILLY AND COMPANY, PATENT DIVISION, P.O. BOX 6288, INDIANAPOLIS, IN, 46206-6288, US

(21) Appl. No.: 10/203,436

(22) PCT Filed: Jan. 16, 2001
(86) PCT No.: PCT/US01/00007

(51) Int. Cl. A61K 31/403; A61K 31/415; A61K 31/405; A61K 31/40; A61K 31/165

(52) U.S. Cl. 514/406; 514/411; 514/408; 514/419; 514/617

(57) ABSTRACT

A method is disclosed for the treatment of of the symptoms associated with renal dysfunction by administering to an animal in need thereof a therapeutically effective amount of a sPLA₂ inhibitor, such as a 1H-indole-3-glyoxylamide.

### FIELD OF THE INVENTION

The present invention is directed to a method for treating renal dysfunction. More specifically, the present invention is directed to a method for treating the symptoms of renal dysfunction by administering a therapeutically effective amount of a sPLA₂ inhibitor.

### BACKGROUND OF THE INVENTION

Renal failure can be either acute or chronic. Acute renal failure (ARF) is a very common renal disease affecting about 5% of all hospitalized patients. ARF carries a high mortality of more than 50% and there has been no significant change in the mortality rate over the past four decades. The effects of acute renal failure may be reversible with prompt and proper intervention and treatment.

Chronic renal failure on the other hand is often not reversible. Common initiating causes of chronic renal failure (CRF) include glomerulonephritis, diabetes mellitus, and hypertension. CRF is characterized by progressive reduction in nephron mass, which eventually impacts the operation of virtually every major organ in the body. The clinical syndrome that results from profound loss of renal function is called uremia. The term encompasses more than just a presumed retention of urea and other toxins but includes the impairment of a host of metabolic and endocrine functions normally subserved by the kidney. Symptoms of uremia include for example, hypothermia-caused possibly by retention of urea and other toxins, impaired glucose metabolism, uremic toxicity caused by an inability to eliminate end products of protein metabolism, fluid and electrolyte imbalance, gastrointestinal disturbances e.g. nausea, anorexia, uremic factors, and peptic ulcer. Other symptoms of CRF include arterial hypertension, congestive heart failure, dermatological disturbances, e.g. itching, neurologic disturbances e.g. impaired mentation, headache, seizures, and immunological disturbances such as for example, leukopenia, lymphocytopenia, bleeding diathesis. (See generally, Harrisson's Principles of Internal Medicine, Isselbacher, K. J. et. al., editors, pages 1259-1284, 13th edition, 1994, McGraw-Hill Publishers, NY, New York.) Because there is neither clear understanding of the cause nor the mechanism of action of ARF, CRF or any of the related renal dysfunctional problems, numerous scientific publications have posited theories.

One theory examines the controversial involvement of sPLA₂-induced cell membrane damage (Edelstein, C. L.;

Ling, H.; Schrier, R. W.; International Society of Nephrology, 51, 1997, 1341-1351). Edelstein et al. propose a dual mode of action for sPLA₂ and sPLA₂ inhibitors because both cell death and cellular protection are seemingly involved.

U.S. Pat. No. 5,654,326 which is incorporated herein by reference, describes 1H-indole-3-glyoxylamide sPLA₂ inhibitors for use in inhibiting sPLA₂ mediated release of fatty acids in conditions such as septic shock. Major therapies for renal dysfunction include the following:

1. Dialysis treatment to remove harmful toxins

2. Drugs to restore salt and water balance, e.g., diuretics

3. Antibiotics

4. Inhaled beta-adrenergic agonists

However, previous treatment attempts have focused on pharmaceutical agents that treated the cause of the renal dysfunction. For example, previous methods treated the problem causing renal dysfunction and not, for example, the build up of fluids or cell membrane damage.

Also of importance are the problems relative to renal organ transplant. Transplantation of kidneys and associated tissues creates additional sepsis and tissue rejection concerns. M. Baur et al. have reported increased PLA levels in patients undergoing major surgery (Baur, M.; Schmid, T. O.; Landauer, B.; Klin. Wochenschr., Vol. 67(3), 1989. Baur et al. further observed that serum PLA levels decrease spontaneously in recovering postsurgical patients.

Accordingly, there is a substantial need for an effective treatment for the many symptoms secondary to renal dysfunction including sepsis, inflammation, cell membrane damage, secondary to organ failure and tissue rejection following kidney transplant.

### SUMMARY OF THE INVENTION

The present invention is directed to a method of alleviating the symptoms of an animal afflicted with renal dysfunction e.g., acute or chronic renal failure, by administering a therapeutically effective amount of a sPLA₂ inhibitor compound.

The present invention also relates to the use of a sPLA₂ inhibitor in combination with a therapeutically effective agent and or medical procedure selected from the group consisting of, dialysis treatment to remove harmful toxins; drugs to restore salt and water balance; and/or other treatment of acute and chronic renal failure.

The invention also relates to the combination of a sPLA₂ inhibitor with ANF atrial naturetic factor for the delay or prevention of acute renal failure in a mammal.

This invention also relates to the use of a sPLA₂ inhibitor in combination with erythropoetin to stimulate red cell production in a mammal.

The present invention is also the use of a sPLA₂ inhibitor in combination with OKT3™ (a monoclonal antibody used to prevent graft rejection by T3 antigens produced by human T cells), to reduce the symptoms associated with administration of OKT3.

### DETAILED DESCRIPTION OF THE INVENTION

Definitions:

General Definitions:

As used herein “Renal dysfunction” means a disease state or condition wherein the renal tissues fail to perform their normal functions according to competent medical authority. Renal dysfunction shall also include long term or chronic renal dysfunction also known as renal failure. Renal dysfunction also includes acute renal failure and disease states that lead to renal failure. Examples of disease states leading to renal failure include for example, acute nephritis, nephrotic syndrome, pyuria, auria, oliguria, uremia, bilateral arterial occlusion, acute tubular necrosis, acute uric acid nephropathy, hypovolemia, acute bilateral upper tract obstruction, hypocalcemic nephropathy, hemolytic uremic syndrome, acute urinary retention, scleroderma, hypersensitivity nephropathy, malignant nephrosclerosis, essential and mixed cryoimmunoglobulinemia, and azotemia.

The term “uremia” means the clinical syndrome of nausea, itching, weight loss, sleep disturbances, altered mentation, and memory loss associated with renal dysfunction.

The terms “sPLA₂ inhibitor” and “sPLA₂ inhibitor compound” as used herein are synonymous.

The term “therapeutically effective amount” is a quantity of sPLA₂ inhibitor sufficient to ameliorate the symptoms secondary to renal dysfunction in an animal. The term “therapeutically effective interval” is a period of time beginning when one of either the sPLA₂ inhibitor or the co-agent is administered or practiced on the patient in need thereof, and ending at the limit of the therapeutic effectiveness of either or both.

The terms “parenteral” or “parenteral administration” mean administration by a route such as subcutaneous, intramuscular, intraorbital, intracapsular, intraspinal, intrasternal, transdermal, transmucosal, transbuccal, transrectal, transvaginal, transnasal or intravenous.

The term “animal” means any member of the animal kingdom including mammals, reptiles, fishes and fowls.

The term “active compound” means one or more sPLA₂ inhibitors used in the method of the invention.

The term “in combination with” denotes the co-administration of a sPLA₂ inhibitor and a co-agent therapy or procedure. The term further means simultaneous co-administration either as a single formulation or as separate formulations or sequential administration of a sPLA₂ inhibitor and co-agent or other therapeutically effective procedure.

The term “co-agent” is a theraputically effective medication or procedure administered in combination with a sPLA₂ inhibitor either as a single dose unit or as separate dose units simultaneously or sequentially within a therapeutic interval.

Preferred Aspects of the Invention

A preferred aspect of the invention is the use of a sPLA₂ inhibitor compound of the invention for the prevention or treatment of uremia. Preclinical studies showed that monkeys and rabbits in renal failure did not develop signs of uremia i.e., the kidneys were failing but the animals were not as clinically ill as they should have been.

Another preferred aspect of the present invention is the use of a sPLA₂ inhibitor in combination with an anti-rejection drug, e.g., OKT3™ for the treatment of chronic or acute inflammation associated with kidney transplant therapy.

Also preferred is the use of sPLA₂ inhibitor in combination with erythropoietin to stimulate the production of red cells in renal failure patients.

I. sPLA₂ Inhibitors Useful in the Method of the Invention

Secretary phopholipase A₂ (sPLA₂) inhibitors in general are useful in the practice of the method of this invention. Exemplary of classes of suitable sPLA₂ inhibitors useful in the method of the invention for treatment of renal dysfunction includes members selected from the group comprising: 1H-indole-3-glyoxylamide, 1H-indole-3-hydrazide, 1H-indole-3-acetamide, 1H-indole-1-glyoxylamide, 1H-indole-1-hydrazide, 1H-indole-1-acetamide, indolizine-1-acetamide, indolizine-1-acetic acid hydrazide, indolizine-1-glyoxylamide, indene-1-acetamide, indene-1-acetic acid hydrazide, indene-1-glyoxylamide, carbazole, tetrahydrocarbazole, pyrazole, phenyl glyoxamide, pyrrole, naphthyl glyoxamide, naphthyl acetamide, phenyl acetamide, pyrrolo[1,2-a]pyrazine, 9H-carbazole, 9-benzylcarbazole and mixtures thereof.

Each of the above sPLA₂ inhibitor types is discussed in the following sections (a) through (n) wherein details of their molecular configuration are given along with methods for their preparation.

A) The 1H—Indole-3-Glyoxylamide Inhibitors

The 1H-indole-3-glyoxylamide sPLA₂ inhibitors and method of making them are described in U.S. Pat. No. 5,654,326, the disclosure of which is incorporated herein by reference. These 1H-indole-3-glyoxylamide compounds are also described in European Patent Application No. 95302166.4, Publication No. 0675110 (publ., Oct. 4, 1995).

Definitions For 1H-Indole-3-Glyoxylamide Compounds:

The words, “acid linker” refers to a divalent linking group of the 1H-indole-3-glyoxylamide compounds is symbolized as, -(Lₐ)-, which has the function of joining the 4 or 5 position of the indole nucleus to an acidic group in the general relationship:

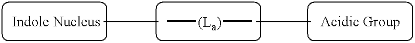

The words, “acid linker length”, refer to the number of atoms (excluding hydrogen) in the shortest chain of the linking group -(Lₐ)- that connects the 4 or 5 position of the indole nucleus with the acidic group.

### DETAILED DESCRIPTION OF THE INVENTION

The method of the invention includes a method for treatment of an animal afflicted with renal dysfunction.

This method includes administering to said animal a therapeutically effective amount of a 1H-indole-3-glyoxylamide represented by the formula (I), or a pharmaceutically acceptable salt or aliphatic ester prodrug derivative thereof;

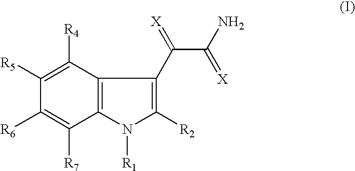

where;

X is oxygen,

R¹ is selected from the group consisting of —C₇-C₂₀ alkyl,

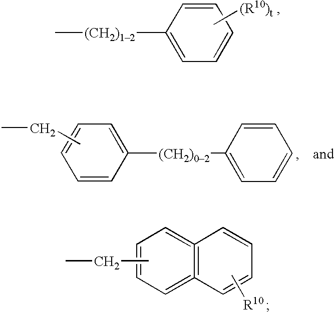

where

R¹⁰ is selected from the group consisting of halo, C₁-C₁₀ alkyl, C₁-C₁₀ alkoxy, —S—(C₁-C₁₀ alkyl) and halo(C₁-C₁₀)alkyl, and t is an integer from 0 to 5 both inclusive;

R₂ is selected from the group consisting of hydrogen, halo, cyclopropyl, methyl, ethyl, and propyl;

R₄ and R₅ are independently selected from the group consisting of hydrogen, a non-interfering substituent and the group, -(Lₐ)-(acidic group); where, at least one of R₄ and R₅ is the group, -(Lₐ)-(acidic group) and wherein the (acidic group) is selected from the group consisting of —CO₂H, —SO₃H, or —P(O) (OH)₂; where,

-(Lₐ)- is an acid linker with the proviso that;

the acid linker group, -(Lₐ)-, for R₄ is selected from the group consisting of

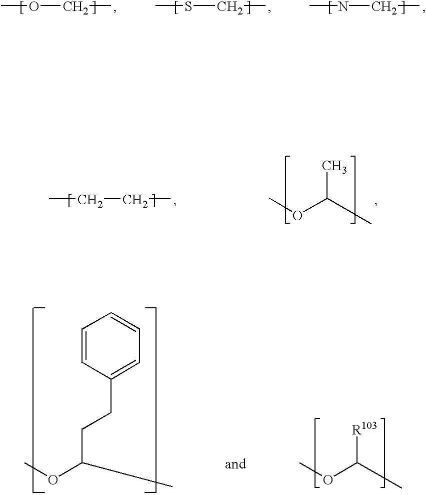

where R¹⁰³ is a non-interfering substituent,

and where,

the acid linker, -(Lₐ)-, for R₅ is selected from the group consisting of

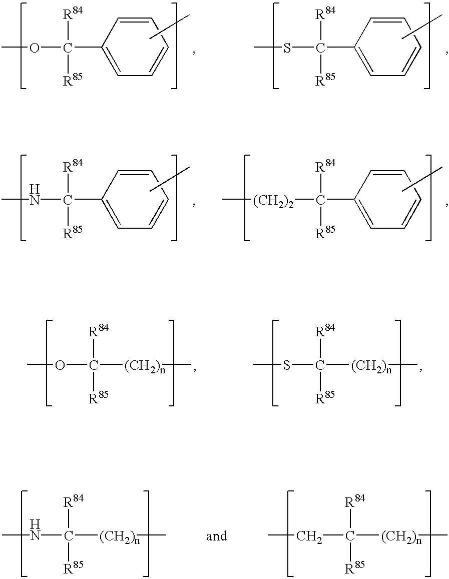

where R⁸⁴ and R⁸⁵ are each independently selected from hydrogen, C₁-C₁₀ alkyl, aryl, C₁-C₁₀ alkaryl, C₁-C₁₀ arylkyl, carboxy, carbalkoxy, and halo and,

R₆ and R₇ are each independently selected from hydrogen and non-interfering substituents, where non-interfering substituents are selected from the group consisting of C₁-C₆ alkyl, C₂-C₆ alkenyl, C₂-C₆ alkynyl, C₇-C₁₂ arylenalkyl, C₇-C₁₂ alkaryl, C₃-C₈ cycloalkyl, C₃-C₈ cycloalkenyl, phenyl, tolulyl, xylenyl, biphenyl, C₁-C6 alkoxy, C₂-C₆ alkenyloxy, C₂-C₆ alkynyloxy, C₂-C₁₂ alkoxyalkyl, C₂-C₁₂ alkoxyalkyloxy, C₂-C₁₂ alkylcarbonyl, C₂-C₁₂ alkylcarbonylamino, C₂-C₁₂ alkoxyamino, C₂-C₁₂ alkoxyaminocarbonyl, C₂-C₁₂ alkylamino, C₁-C₆ alkylthio, C₂-C₁₂ alkylthiocarbonyl, C₁-C₆ alkylsulfinyl, C₁-C₆ alkylsulfonyl, C₂-C₆ haloalkoxy, C₁-C₆ haloalkylsulfonyl, C₂-C₆ haloalkyl, C₁-C₆ hydroxyalkyl, —C(O)O(C₁-C₆ alkyl), —(CH₂)ₙ—O—(C₁-C₆ alkyl), benzyloxy, phenoxy, phenylthio, —(CONHSO₂R), —CHO, amino, amidino, bromo, carbamyl, carboxyl, carbalkoxy, —(CH₂)ₙ—CO₂H, chloro, cyano, cyanoguanidinyl, fluoro, guanidino, hydrazide, hydrazino, hydrazido, hydroxy, hydroxyamino, iodo, nitro, phosphono, —SO₃H, thioacetal, thiocarbonyl, and C₁-C₆ carbonyl and where n is between 1 and 8.

The method of the invention also includes treatment of an animal afflicted by renal dysfunction. This method includes administering to said animal a therapeutically effective amount of a 9H-carbazole compound represented by the formula (II), or a pharmaceutically acceptable salt or aliphatic ester prodrug derivative thereof;

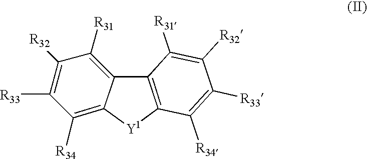

where Y₁ is selected from the group consisting of O, NH, NR¹ and S;

R¹ is selected from the group consisting of —C₇-C₂₀ alkyl,

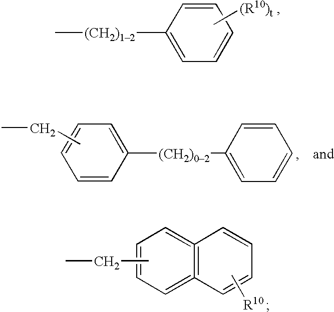

where

R¹⁰ is selected from the group consisting of halo, C₁-C₁₀ alkyl, C₁-C₁₀ alkoxy, —S—(C₁-C₁₀ alkyl) and halo(C₁-C₁₀)alkyl, and t is an integer from 0 to 5 both inclusive;

where R₃₁, R₃₂, R₃₃, R₃₁′, R₃₂′, R₃₃′, R₃₄ and R₃₄, are independently selected from the group consisting of hydrogen, CONR¹⁰¹R¹⁰², alkyl, alkylaryl, aryl, alkylheteroaryl, haloalkyl, alkylCONR¹⁰¹ R¹⁰², a non-interfering substituent and the group,

-(Lₐ)-(acidic group);

where -(Lₐ)- is an acid linker selected from the group consisting of

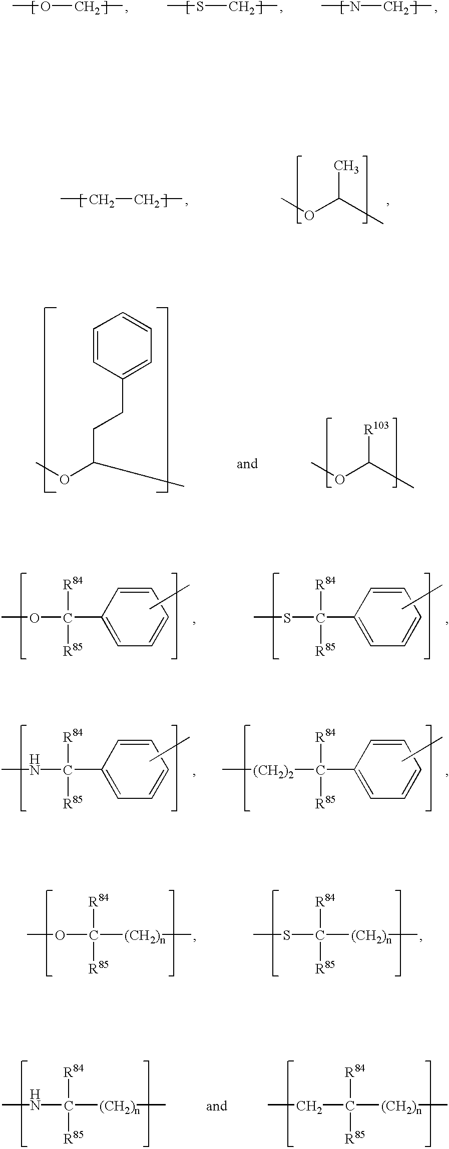

where R⁸⁴ and R⁸⁵ are each independently selected from the group consisting of hydrogen, C₁-C₁₀ alkyl, aryl, C₁-C₁₀ alkaryl, -C₁₀ aralkyl, carboxy, carbalkoxy, and halo; 2 and,

where the (acidic group) is selected from the group consisting of —CO₂H, —SO₃H, —CO₂NR⁰¹R¹⁰² and —P(O)(OH)₂ and,

where R¹⁰¹ and R¹⁰² are independently selected from the group consisting of hydrogen, alkyl, aryl, heteroaryl and haloalkyl and,

where non-interfering substituents are selected from the group consisting of C₁-C₆ alkyl, C₂-C₆ alkenyl, C₂-C₆ alkynyl, C₇-C₁₂ arylalkyl, C₇-C₁₂ alkylaryl, C₃-C₈ cycloalkyl, C₃-C₈ cycloalkyl, phenyl, tolulyl, xylyl, biphenyl, C₁-C₆ alkoxy, C₂-C₆ alkyloxy, C₂-C₆ alkynyloxy, C₂-C₁₂ alkoxyalkyl, C₂-C₁₂ alkoxyalkyloxy, C₂-C₁₂ alkylcarbonyl, C₂-C₁₂ alkylcarbonylamino, C₂-C₁₂ alkoxyamino, C₂-C₁₂ alkoxyaminocarbonyl, C₂-C₁₂ alkylamino, C₁-C₆ alkylthio, C₂-C₁₂ alkylthiocarbonyl, C₁-C₆ alkylsulfinyl, C₁-C₆ alkylsulfonyl, C₂-C₆ haloalkoxy, C₁-C₆ haloalkylsulfonyl, C₂-C₆ haloalkyl, C₁-C₆ hydroxyalkyl, —C(O)O(C₁-C₆ alkyl), —(CH₂)ₙ—O—(C₁-C₆ alkyl), benzyloxy, phenoxy, phenylthio, —(CONHSO₂(R)), —CHO, amino, amidino, bromo, carbamyl, carboxyl, carbalkoxy, —(CH₂)ₙ—CO₂H, chloro, cyano, cyanoguanidinyl, fluoro, guanidino, hydrazide, hydrazino, hydrazido, hydroxy, hydroxyamino, iodo, nitro, phosphono, —SO₃H, thioacetal, thiocarbonyl, and C₁-C₆ carbonyl and where n is between about 1 and 8 and,

R is selected from the group consisting of hydrogen and alkyl and, where at least one of R₃₁, R₃₂, R₃₃ or R₃₄ is the group -(Lₐ)-(acidic group).

The method of the invention also includes treatment of an animal afflicted with renal dysfunction. This method includes administering to said animal in need of such treatment, a therapeutically effective amount of a 1H-indole-3-glyoxylamide compound or a 9H-carbazole or a pharmaceutically acceptable salt, solvate, or a prodrug derivative thereof selected from the group consisting of compounds (A) through (AL):

(A) [[3-(2-amino-1,2-dioxoethyl)-2-methyl-1-(phenylmethyl)-1H indol-4-yl]oxy]acetic acid,

(B) dl-2-[[3-(2-amino-1,2-dioxoethyl)-2-methyl-1-(phenylmethyl)-1H indol-4-yl]oxylpropanoic acid,

(C) [[3-(2-amino-1,2-dioxoethyl)-1-([1,1′-biphenyl]-2-ylmethyl)-2 methyl-1H-indol-4-yl]oxy]acetic acid,

(D) [[3-(2-amino-1,2-dioxoethyl)-1-([1,1′-biphenyl]-3-ylmethyl)-2-methyl-1H-indol-4-yl]oxy]acetic acid,

(E) [[3-(2-amino-1,2-dioxoethyl)-1-([1,1′-biphenyl]-4-ylmethyl)-2-methyl-1H-indol-4-yl]oxy]acetic acid,

(F) [[3-(2-amino-1,2-dioxoethyl)-1-[(2,6-dichlorophenyl)methyl]-2-methyl-1H-indol-4-yl]oxy]acetic acid

(G) [[3-(2-amino-1,2-dioxoethyl)-1-[4(-fluorophenyl)methyl]-2-methyl-1H-indol-4-yl]oxy]acetic acid,

(H) [[3-(2-amino-1,2-dioxoethyl)-2-methyl-1-[(1-naphthalenyl)methyl]-1H-indol-4-yl]oxy]acetic acid,

(I) [[3-(2-amino-1,2-dioxoethyl)-2-ethyl-1-(phenylmethyl)-1H-indol-4-yl]oxy]acetic acid,

(J) [[3-(2-amino-1,2-dioxoethyl)-1-[(3-chlorophenyl)methyl]-2-ethyl-1H-indol-4-yl]oxy]acetic acid,

(K) [[3-(2-amino-1,2-dioxoethyl)-1-([1,1′-biphenyl]-2-ylmethyl)-2-ethyl-1H-indol-4-yl]oxy]acetic acid,

(L) [[3-(2-amino-1,2-dioxoethyl)-1-([1,1′-biphenyl]-2-ylmethyl)-2-propyl-1H-indol-4-yl]oxy]acetic acid,

(M) [[3-(2-amino-1,2-dioxoethyl)-2-cyclopropyl-1-(phenylmethyl)-1H-indol-4-yl]oxy]acetic acid,

(N) [[3-(2-amino-1,2-dioxoethyl)-1-([1,1′-biphenyl]-2-ylmethyl)-2-cyclopropyl-1H-indol-4-yl]oxy]acetic acid,

(O) 4-[[3-(2-amino-1,2-dioxoethyl)-2-ethyl-1-(phenylmethyl)-1H-indol-5-yl]oxy]butanoic acid,

(P) 9H-carbazole,

(Q) 9-benzylcarbazole,

(AG) 1-(9H-benzylcarbazol-1-halo-4-yloxy-5-alkylamido) alkylacetate,

(AH) 1-(9H-benzylcarbazol-4-yloxy-5-alkylamido) alkylacetate,

(AI) 1-(9H-benzylcarbazol-1-halo-4-yloxy-5-alkylamido) acetic acid,

(AJ) 1-(9H-benzylcarbazol-4-yloxy-5-alkylamido) acetic acid and

(AK) mixtures of (AG) through (AJ) and

(AL) mixtures of (A) through (AK) combined with an additional treatment composition.

Particularly useful prodrugs of the compounds of formula (II) and named compounds (A) thru (AL) are the simple aromatic and aliphatic esters, such as the methyl ester.

The invention is a method for treatment of an animal afflicted with renal dysfunction. This method includes administering to said animal in need of such treatment a therapeutically effective amount of a composition selected from the group comprising:

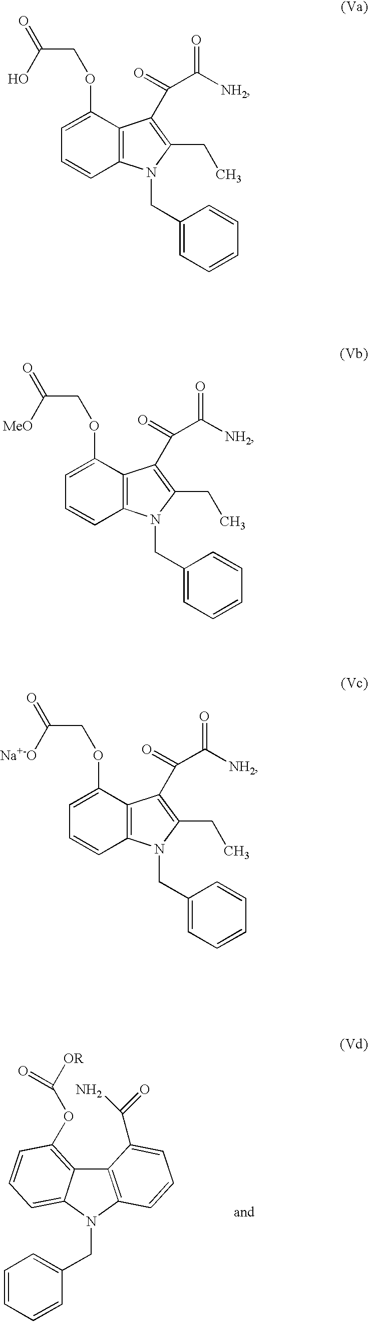

where R is independently selected from the group consisting of hydrogen, alkyl, aryl and heteroaryl.

Preparation of sPLA₂ Inhibitors

The methyl ester of (Va)

a) The 1H-indole-3-glyoxylamide sPLA₂ inhibitors and method of making them are described in U.S. Pat. No. 5,654,326, the entire disclosure of which is incorporated herein by reference. Another method of making 1H-indole-3-glyoxylamide sPLA₂ inhibitors is described in U.S. patent application Ser. No. 09/105,381, filed Jun. 26, 1998 and titled, “Process for Preparing 4-substituted 1-H-Indole-3-glyoxyamides” the entire disclosure of which is incorporated herein by reference. U.S. patent application Ser. No. 09/105,381 discloses the following process having steps (a) thru (i):

preparing a compound of the formula I or a pharmaceutically acceptable salt or prodrug derivative thereof

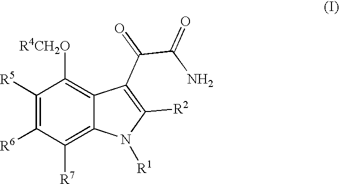

wherein:

R¹ is selected from the group consisting of —C₇-C₂₀ alkyl,

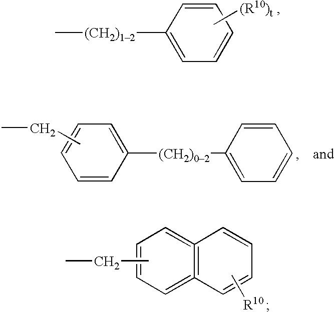

where

R¹⁰ is selected from the group consisting of halo, C₁-C₁₀ alkyl, C₁-C₁₀ alkoxy, —S—(C₁-C₁₀ alkyl) and halo(C₁-C₁₀)alkyl, and t is an integer from 0 to 5 both inclusive;

R² is selected from the group consisting of hydrogen, halo, C₁-C₃ alkyl, C₃-C₄ cycloalkyl, C₃-C₄ cycloalkenyl, —O—(C₁-C₂ alkyl), —S—(C₁-C₂ alkyl), aryl, aryloxy and HET;

R⁴ is selected from the group consisting of —CO₂H, —SO₃H and —P(O)(OH)₂ or salt and prodrug derivatives thereof; and

R⁵, R⁶ and R⁷ are each independently selected from the group consisting of hydrogen, (C₁-C₆)alkyl, (C₁-C₆)alkoxy, halo(C₁-C₆)alkoxy, halo(C₂-C₆)alkyl, bromo, chloro, fluoro, iodo and aryl;

which process comprises the steps of:

a) halogenating a compound of formula X

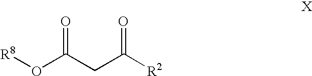

where R⁸ is (C₁-C₆)alkyl, aryl or HET;

with SO₂Cl₂ to form a compound of formula IX

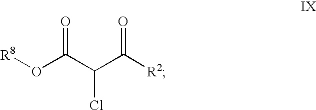

b) Hydrolyzing and decarboxylating a compound of formula IX

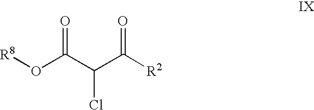

to form a compound of formula VIII

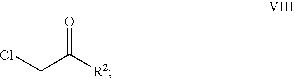

c) alkylating a compound of formula VII

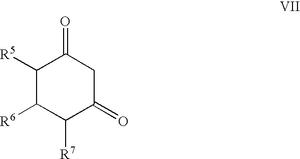

with a compound of formula VIII

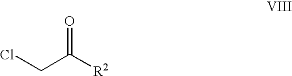

to form a compound of formula VI

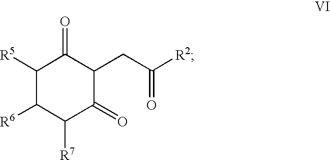

d) aminating and dehydrating a compound of formula VI

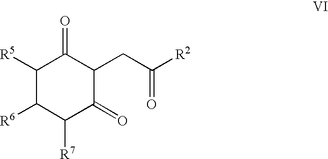

with an amine of the formula R¹NH₂ in the presence of a solvent that forms and azeotrope with water to form a compound of formula V;

e) oxidizing a compound of formula V

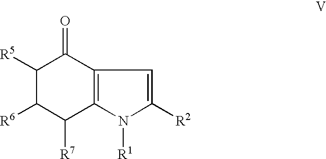

by refluxing in a polar hydrocarbon solvent having a boiling point of at least 150° C. and a dielectric constant of at least 10 in the presence of a catalyst to form a compound of formula IV

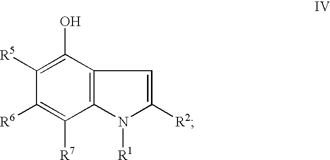

f) alkylating a compound of the formula IV

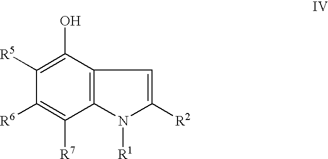

with an alkylating agent of the formula XCH₂R^{4a} where X is a leaving group and R^{4a} is —CO₂R^{4b}, —SO₃R^{4b}, —P(O) (OR^{4b})₂, or —P(O) (OR^{4b})H, where R^{4b} is an acid protecting group to form a compound of formula III

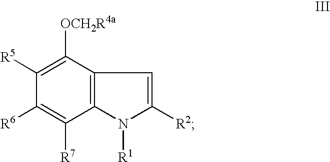

g) reacting a compound of formula III

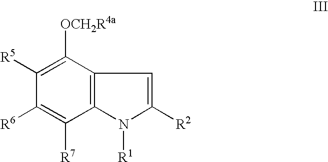

with oxalyl chloride and ammonia to form a compound of formula II

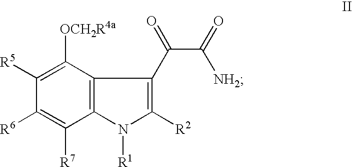

h) optionally hydrolyzing a compound of formula II

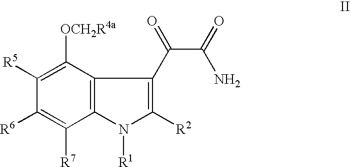

to form a compound of formula I; and

i) optionally salifying a compound of formula I.

The synthesis methodology for making the 1H-indole-3-glyoxylamide sPLA₂ inhibitor starting material may be by any suitable means available to one skilled in the chemical arts. However, such methodology is not part of the present invention which is a method of use, specifically, a method of treating mammal afflicted with or susceptible to renal dysfunction.

The method of the invention is for treatment of a mammal, including a human, afflicted with renal dysfunction, said method comprising administering to said human a therapeutically effective amount of the compound represented by formula (Ia), or a pharmaceutically acceptable salt or prodrug derivative thereof;

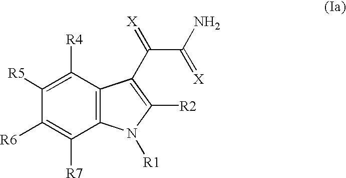

wherein

Both X are oxygen;

R¹ is selected from the group consisting of

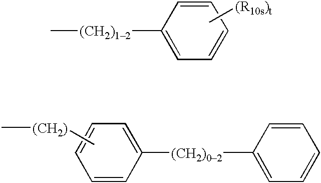

Where R¹⁰ is a radical independently selected from halo, C₁-C₁₀ alkyl, C₁-C₁₀ alkoxy, —S—(C₁-C₁₀ alkyl), and C₁-C₁₀ haloalkyl and t is a number from 0 to 5;

R₂ is selected from the group; halo, cyclopropyl, methyl, ethyl, and propyl;

R₄ and R₅ are independently selected from hydrogen, a non-interfering substituent, or the group, -(Lₐ)-(acidic group); wherein -(Lₐ)- is an acid linker;

provided, the acid linker group, -(Lₐ)-, for R₄ is selected from the group consisting of;

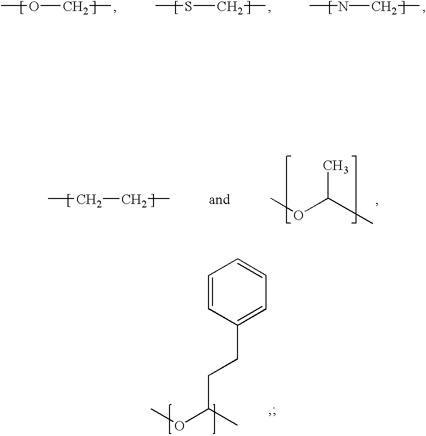

and provided, the acid linker, -(Lₐ)-, for R₅ is selected from group consisting of;

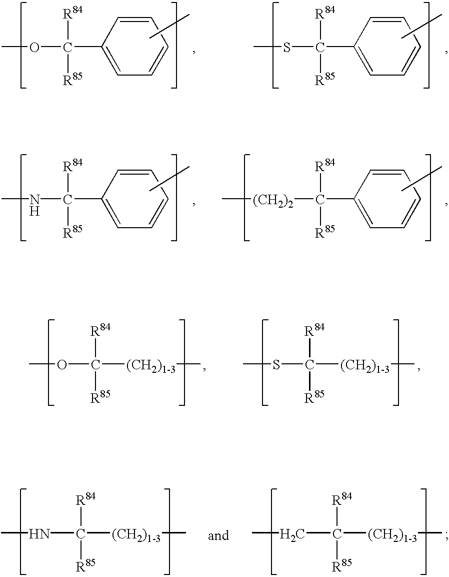

wherein R₈₄ and R₈₅ are each independently selected from hydrogen, C₁-C₁₀ alkyl, aryl, C₁-C₁₀ alkaryl, C₁-C₁₀aralkyl, carboxy, carbalkoxy, and halo; and

provided, that at least one of R₄ and R₅ must be the group, -(Lₐ)-(acidic group) and wherein the (acidic group) on the group -(Lₐ)-(acidic group) of R₄ or R₅ is selected from —CO₂H, —SO₃H, or —P(O)(OH)₂;

R₆ and R₇ are each independently selected from hydrogen and non-interfering substituents, with the non-interfering substituents being selected from the group consisting of the following: C₁-C₆ alkyl, C₂-C₆ alkenyl, C₂-C₆ alkynyl, C₇-C₁₂ aralkyl, C₇-C₁₂ alkaryl, C₃-C₈ cycloalkyl, C₃-C₈ cycloalkenyl, phenyl, tolulyl, xylenyl, biphenyl, C₁-C₆ alkoxy, C₂-C₆ alkenyloxy, C₂-C₆ alkynyloxy, C₂-C₁₂ alkoxyalkyl, C₂-C₁₂ alkoxyalkyloxy, C₂-C₁₂ alkylcarbonyl, C₂-C₁₂ alkylcarbonylamino, C₂-C₁₂ alkoxyamino, C₂-C₁₂ alkoxyaminocarbonyl, C₂-C₁₂ alkylaimino, C₁-C₆ alkylthio, C₂-C₁₂ alkylthiocarbonyl, C₁-C₆ alkylsulfinyl, C₁-C₆ alkylsulfonyl, C₂-C₆ haloalkoxy, C₁-C₆ haloalkylsulfonyl, C₂-C₆ haloalkyl, C₁-C₆ hydroxyalkyl, —C(O)O(C₁-C₆ alkyl), —(CH₂)ₙ, —(C₁-C₆ alkyl), benzyloxy, phenoxy, phenylthio, —(CONHSO₂R), —CHO, amino, amidino, bromo, carbamyl, carboxyl, carbalkoxy, —(CH₂)ₙ—CO₂H, chloro, cyano, cyanoguanidinyl, fluoro, guanidino, hydrazide, hydrazino, hydrazido, hydroxy, hydroxyamino, iodo, nitro, phosphono, —SO₃H, thioacetal, thiocarbonyl, and C₁-C₆ carbonyl; where n is from 1 to 8.

Preferred for practicing the method of the invention are 1H-indole-3-glyoxylamide compounds and all corresponding pharmaceutically acceptable salts, solvates and prodrug derivatives thereof which are useful in the method of the invention include the following:

(A) [[3-(2-Amino-1,2-dioxoethyl)-2-methyl-1-(phenylmethyl)-1H-indol-4-yl]oxy]acetic acid,

(B) dl-2-[[3-(2-Amino-1,2-dioxoethyl)-2-methyl-1-(phenylmethyl)-1H-indol-4-yl]oxy]propanoic acid,

(C) [[3-(2-Amino-1,2-dioxoethyl)-1-([1,1′-biphenyl]-2-ylmethyl)-2-methyl-1H-indol-4-yl]oxy]acetic acid,

(D) [[3-(2-Amino-1,2-dioxoethyl)-1-([1,1′-biphenyl]-3-ylmethyl)-2-methyl-1H-indol-4-yl]oxy]acetic acid,

(E) [[3-(2-Amino-1,2-dioxoethyl)-1-([1,1′-biphenyl]-4-ylmethyl)-2-methyl-1H-indol-4-yl]oxy]acetic acid,

(F) [[3-(2-Amino-1,2-dioxoethyl)-1-[(2,6-dichlorophenyl)methyl]-2-methyl-1H-indol-4-yl]oxy]acetic acid

(G) [[3-(2-Amino-1,2-dioxoethyl)-1-[4(-fluorophenyl)methyl]-2-methyl-1H-indol-4-yl]oxy]acetic acid,

(H) [[3-(2-Amino-1,2-dioxoethyl)-2-methyl-1-[(1-naphthalenyl)methyl]-1H-indol-4-yl]oxy]acetic acid, (I) [[3-(2-Amino-1,2-dioxoethyl)-2-ethyl-1-(phenylmethyl)-1H-indol-4-yl]oxy]acetic acid,

(J) [[3-(2-Amino-1,2-dioxoethyl)-1-[(3-chlorophenyl)methyl]-2-ethyl-1H-indol-4-yl]oxy]acetic acid,

(K) [[3-(2-Amino-1,2-dioxoethyl)-1-([1,1′-biphenyl]-2-ylmethyl)-2-ethyl-1H-indol-4-yl]oxy]acetic acid,

(L) [[3-(2-amino-1,2-dioxoethyl)-1-([1,1′-biphenyl]-2-ylmethyl)-2-propyl-1H-indol-4-yl]oxy]acetic acid,

(M) [[3-(2-Amino-1,2-dioxoethyl)-2-cyclopropyl-1-(phenylmethyl)-1H-indol-4-yl]oxy]acetic acid,

(N) [[3-(2-Amino-1,2-dioxoethyl)-1-([1,1′-biphenyl]-2-ylmethyl)-2-cyclopropyl-1H-indol-4-yl]oxy]acetic acid,

(O) 4-[[3-(2-Amino-1,2-dioxoethyl)-2-ethyl-1-(phenylmethyl)-1H-indol-5-yl]oxy]butanoic acid,

(P) mixtures of (A) through (P) in any combination.

Particularly useful prodrugs of the compounds of formula (I) and named compounds (A) thru (O) are the simple aromatic and aliphatic esters, such as the methyl ester, ethyl ester, n-propyl ester, isopropyl ester, n-butyl ester, sec-butyl, tert-butyl ester, N,N-diethylglycolamido ester, and morpholino-N-ethyl ester. Methods of making ester prodrugs are disclosed in U.S. Pat. No. 5,654,326. Additional methods of prodrug synthesis are disclosed in U.S. Provisional Patent Application Serial No. 60/063,280 filed Oct. 27, 1997 (titled, N,N-diethylglycolamido ester Prodrugs of Indole sPLA₂ Inhibitors), the entire disclosure of which is incorporated herein by reference; U.S. Provisional Patent Application Serial No. 60/063,646 filed Oct. 27, 1997 (titled, Morpholino-N-ethyl Ester Prodrugs of Indole sPLA₂ Inhibitors), the entire disclosure of which is incorporated herein by reference; and U.S. Provisional Patent Application Serial No. 60/063,284 filed Oct. 27, 1997 (titled, Isopropyl Ester Prodrugs of Indole sPLA₂ Inhibitors), the entire disclosure of which is incorporated herein by reference.

Most preferred in the practice of the method of the invention are the acid, sodium salt, methyl ester, and morpholino-N-ethyl ester forms of [[3-(2-amino-1,2-dioxoethyl)-2-ethyl-1-(phenylmethyl)-1H-indol-4-yl]oxy]acetic acid as represented by the following formulae:

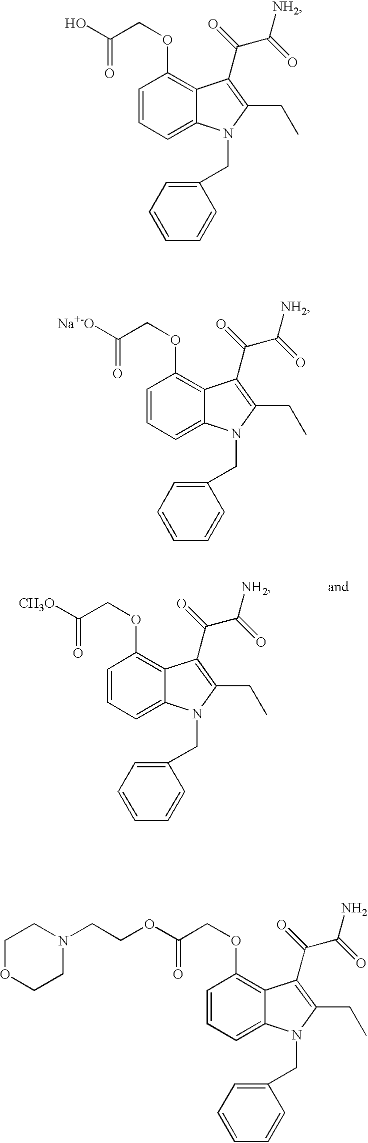

another highly preferred compound is the indole-3-glyoxylamide morpholino ethyl ester of represented by the formula:

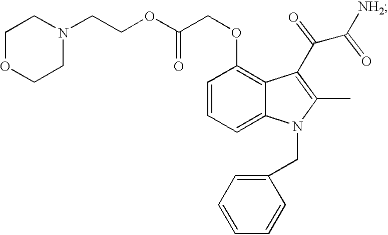

The preparation of which is further described in U.S. provisional patent application SNo. 60/063,646 filed Oct. 27, 1997.

Synthesis methods for 1H-indole-3-glyoxylamide sPLA₂ inhibitors are additionally depicted in the following reaction scheme:

1H-indole-3-glyoxylamide Reaction Scheme

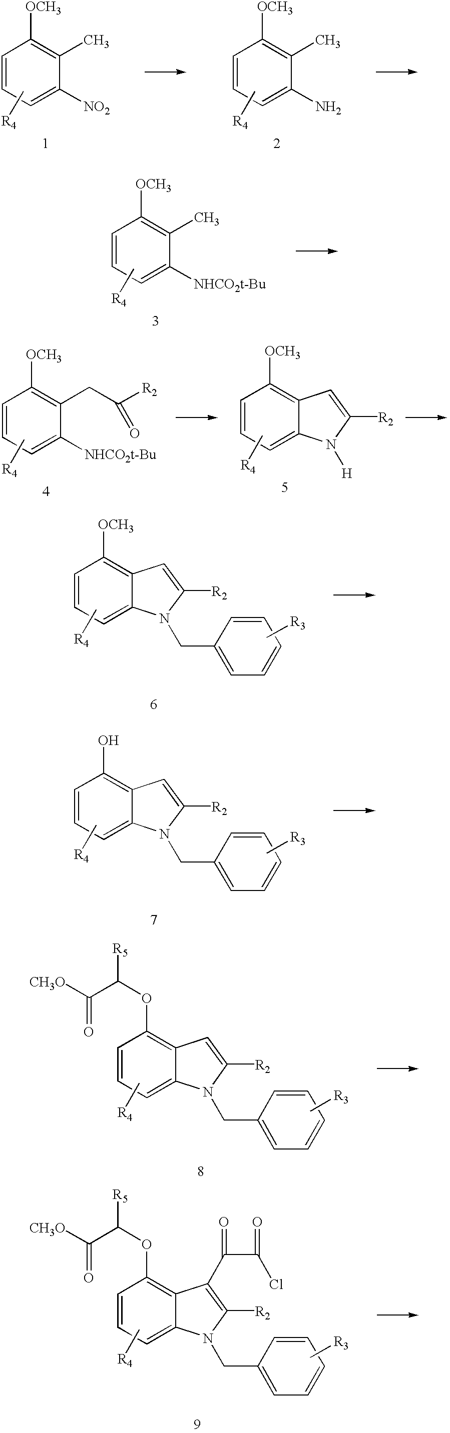

Explanation of Reaction Scheme:

To obtain the glyoxylamides substituted in the 4-position with an acidic function through an oxygen atom, the reactions outlined in scheme 1 are used (for conversions 1 through 5, see Robin D. Clark, Joseph M. Muchowski, Lawrence E. Fisher, Lee A. Flippin, David B. Repke, Michel Souchet, Synthesis, 1991, 871-878, the disclosures of which are incorporated herein by reference. The ortho-nitrotoluene, 1, is readily reduced to the 2-methylaniline, 2, using Pd/C as catalyst. The reduction can be carried out in ethanol or tetrahydrofuran (THF) or a combination of both, using a low pressure of hydrogen. The aniline, 2, on heating with di-tert-butyl dicarbonate in THF at reflux temperature is converted to the N-tert-butylcarbonyl derivative, 3, in good yield. The dilithium salt of the dianion of 3 is generated at −40 to −20° C. in THF using sec-butyl lithium and reacted with the appropriately substituted N-methoxy-N-methylalkanamide. This product, 4, may be purified by crystallization from hexane, or reacted directly with trifluoroacetic acid in methylene chloride to give the 1,3-unsubstituted indole 5. The 1,3-unsubstituted indole 5 is reacted with sodium hydride in dimethylformamide at room temperature (20-25° C.) for 0.5-1.0 hour. The resulting sodium salt of 5 is treated with an equivalent of arylmethyl halide and the mixture stirred at a temperature range of 0-100° C., usually at ambient room temperature, for a period of 4 to 36 hours to give the 1-arylmethylindole, 6. This indole, 6, is O-demethylated by stirring with boron tribromide in methylene chloride for approximately 5 hours (see Tsung-Ying Shem and Charles A Winter, Adv. Drug Res., 1977, 12, 176, the disclosure of which is incorporated herein by reference). The 4-hydroxyindole, 7, is alkylated with an alpha bromoalkanoic acid ester in dimethylformamide (DMF) using sodium hydride as a base, with reactions conditions similar to that described for the conversion of 5 to 6. The a-[(indol-4-yl)oxy]alkanoic acid ester, 8, is reacted with oxalyl chloride in methylene chloride to give 9, which is not purified but reacted directly with ammonia to give the glyoxamide 10. This product is hydrolyzed using 1N sodium hydroxide in methanol. The final glyoxylamide, 11, is isolated either as the free carboxylic acid or as its sodium salt or in both forms.

The most preferred compound, [[3-(2-Amino-1,2-dioxoethyl)-2-ethyl-1-(phenylmethyl)-1H-indol-4-yl]oxy]acetic acid (as well as its sodium salt and methyl ester) useful in the practice of the method of the invention, may be prepared by the following procedure:

preparation of [[3-(2-Amino-1,2-dioxoethyl)-2-ethyl-1-(phenylmethyl)-1H-indol-4-yl]oxy]acetic acid, a compound represented by the formula:

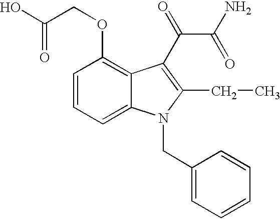

Part A. Preparation of 2-Ethyl-4-methoxy-1H-indole.

A solution of 140 ml (0.18 mol) of 1.3M sec-butyl lithium in cyclohexane is added slowly to N-tert-butoxycarbonyl-3-methoxy-2-methylaniline (21.3 g, 0.09 mol) in 250 ml of THF keeping the temperature below -40° C. with a dry ice-ethanol bath. The bath is removed and the temperature allowed to rise to 0° C. and then the bath replaced. After the temperature has cooled to −60° C., 18.5 g (0.18 mmol) of N-methoxy-N-methylpropanamide in an equal volume of THF iss added dropwise. The reaction mixture is stirred 5 minutes, the cooling bath removed and stirred an additional 18 hours. It is then poured into a mixture of 300 ml of ether and 400 ml of 0.5N HCl. The organic layer is separated, washing with water, brine, dried over MgSO₄, and concentrated at reduced pressure to give 25.5 g of a crude of 1-[2-(tert-butoxycarbonylamino)-6-methoxyphenyl]-2-butanone. This material is dissolved in 250 ml of methylene chloride and 50 ml of trifluoroacetic acid and stirred for a total of 17 hours. The mixture is concentrated at reduced pressure and ethyl acetate and water added to the remaining oil. The ethyl acetate is separated, washed with brine, dried (MgSO₄) and concentrated. The residue is chromatographed three times on silica eluting with 20% EtOAc/hexane to give 13.9 g of 2-ethyl-4-methoxy-1H-indole.

Analysis for C₁₁H₁₃NO:

| | |
|---|---|
| Calculated: | C, 75.40; H, 7.48; N, 7.99; |
| Found: | C, 74.41; H, 7.64; N, 7.97. |

Part B. Preparation of 2-Ethyl-4-methoxy-1-(phenylmethyl)-1H-indole.

2-Ethyl-4-methoxy-1H-indole (4.2 g, 24 mmol) is dissolved in 30 ml of DMF and 960 mg (24 mmol) of 60% NAH/mineral oil is added. After 1.5 hours, 2.9 ml(24 mmol) of benzyl bromide is added. After 4 hours, the mixture is diluted with water extracting twice with ethyl acetate. The combined ethyl acetate is washed with brine, dried (MgSO₄) and concentrated at reduced pressure. The residue is chromatographed on silica gel and eluted with 20% EtOAc/hexane to give 3.1 g (49% yield) of 2-ethyl-4-methoxy-1-(phenylmethyl)-1H-indole.

Part C. Preparation of 2-Ethyl-4-hydroxy-1-(phenylmethyl)-1H-indole.

A solution of 3.1 g (11.7 mmol) of 2-ethyl-4-methoxy-1-(phenylmethyl)-1H-indole and 48.6 ml of 1M BBr₃/CH₂Cl₂ in 50 ml of methylene chloride is stirred at room temperature for 5 hours and concentrated at reduced pressure. The residue is dissolved in ethyl acetate, washed with brine and dried (mgso₄). After concentrating at reduced pressure, the residue is chromatographed on silica gel eluting with 20% EtOAc/hexane to give 1.58 g (54% yield) of 2-ethyl-4-hydroxy-1-(phenylmethyl)-1H-indole, mp, 86-90° C.

Analysis for C₁₇H₁₇NO:

| | |
|---|---|
| Calculated: | C, 81.24; H, 6.82; N, 5.57; |
| Found: | C, 81.08; H, 6.92; N, 5.41. |

Part D. Preparation of [[2-Ethyl-1-(phenylmethyl)-1H-indol-4-yl]oxy]acetic acid tert-butyl ester.

2-Ethyl-4-hydroxy-1-(phenylmethyl)-1H-indole (5.82 g, 20 mmol) is added to 7.82 g (24 mmol) cesium carbonate in 25 ml DMF and the mixture is stirred at 35° C. for 30 minutes. After cooling to 20° C., a solution of tert-butyl bromoacetate (4.65 g, 23.8 mmol) in 5 ml DMF is added and stirring maintained until the reaction is judged complete by TLC analysis (several hours). The mixture is diluted with water and extracted with ethyl acetate. The ethyl acetate solution is washed with brine, dried (MgSO₄) and concentrated at reduced pressure to give 6.8 g of solid.

Mass spectrum: 365

Analyses for C₂₃H₂₇NO₃:

| | |
|---|---|
| Calculated: | C, 75.59; H, 7.75; N, 3.83; |
| Found: | C, 75.87; H, 7.48; N, 3.94. |

Part E. Preparation of [[2-Ethyl-1-(phenylmethyl)-3-ureido-1H-indol-4-yl]oxy]acetic acid tert-butyl ester.

A solution of 2.3 g (6.3 mmol) [[2-ethyl-1-(phenylmethyl)-1H-indol-4-yl]oxy]acetic acid tert-butyl ester and 4.8 g (12.6 mmol) bis(2,2,2-trichloroethyl)-azodicarboxylate in diethyl ether is stirred for 24 hours at room temperature. The resulting solid is filtered and vacuum dried. This adduct (1 g, 1.3 mmol) is dissolved in 10 ml of THF and treated with zinc (1 g) and glacial acetic acid (0.5 ml). After stirring for 30 minutes at room temperature an excess of trimethylsilylisocyanate in 1 ml of THF is added and stirring is continued at room temperature for 18 hours. The mixture is diluted with water and extracted with ethyl acetate. The organic layer is washed with brine, dried over MgSO₄ and concentrated to dryness to give 0.385 g (69% yield) of the subtitled material.

Mass spectrum: 423.

Analyses for C₂₄H₂₉N₃O₄:

| | |
|---|---|
| Calculated: | C, 68.07; H, 6.90; N, 9.92; |
| Found: | C, 67.92; H, 6.84; N, 9.70. |

Part F. Preparation of [[3-(2-Amino-1,2-dioxoethyl)-2-ethyl-1-(phenylmethyl)-1H-indol-4-yl]oxy]acetic acid.

A mixture of 788 mg (2 mmol) of [3-(2-amino-1,2-dioxoethyl)-2-ethyl-1-(phenylmethyl)-1H-indol-4-yl]oxy]-acetic acid methyl ester, 10 ml of in NaOH and 30 ml of methanol is heated to maintain reflux for 0.5 hour, stirred at room temperature for 0.5 hour and concentrated at reduced pressure. The residue is taken up in ethyl acetate and water, the aqueous layer separated and made acidic to pH 2-3 with 1N HCl. The precipitate is filtered and washed with ethyl acetate to give 559 mg (74% yield) of [[3-(2-amino-1,2-dioxoethyl)-2-ethyl-1-(phenylmethyl)-1H-indol-4-yl]oxy]acetic acid, mp, 230-234° C.

Analyses for C₂₁H₂₀N₂O₅:

| | |
|---|---|
| Calculated: | C, 65.96; H, 5.80; N, 7.33; |
| Found: | C, 66.95; H, 5.55; N, 6.99. |

B) 1H-indole-3-hydrazide sPLA₂ inhibitors useful in practicing the method of the invention are described in U.S. Pat. No. 5,578,634; the entire disclosure of which is incorporated herein by reference. The method of the invention is for treatment of a mammal, including a human, afflicted with renal dysfunction, said method comprising administering to said human a therapeutically effective amount of the compounds described as 1H-indole-3-acetic acid hydrazides represented by the formula (Ib), and pharmaceutically acceptable salts, and prodrugs thereof;

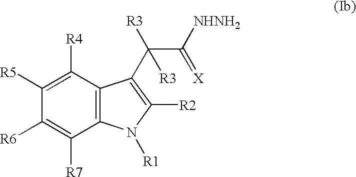

wherein;

X is oxygen or sulfur;

R¹ is selected from groups (i), (ii) and (iii) where;

(i) is C₄-C₂₀ alkyl, C₄-C₂₀ alkenyl, C₄-C₂₀ alkynyl, C₄-C₂₀ haloalkyl, C₄-C₁₂ cycloalkyl, or

(ii) is aryl or aryl substituted by halo, —CN, —CHO, —OH, —SH, C₁-C₁₀ alkylthio, C₁-C₁₀ alkoxy, C₁-C₁₀ alkyl, carboxyl, amino, or hydroxyamino;

(iii) is

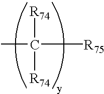

where y is from 1 to 8, R₇₄ is, independently, hydrogen or C₁-C₁₀ alkyl, and R₇₅ is aryl or aryl substituted by halo, —CN, —CHO, —OH, nitro, phenyl, —SH, C₁-C₁₀ alkylthio, C₁-C₁₀ alkoxy, C₁-C₁₀ alkyl, amino, hydroxyamino or a substituted or unsubstituted 5- to 8-membered heterocyclic ring;

R₂ is halo, C₁-C₃ alkyl, ethenyl, C₁-C₂ alkylthio, C₁-C₂ alkoxy, —CHO, —CN;

each R₃ is independently hydrogen, C₁-C₃ alkyl, or halo;

R₄, R₅, R₆, and R₇ are each independently hydrogen, C₁-C₁₀ alkyl, C₁-C₁₀ alkenyl, C₁-C₁₀ alkynyl, C₃-C₈ cycloalkyl, aryl, aralkyl, or any two adjacent hydrocarbyl groups in the set R₄, R₅, R₆, and R₇ combined with the ring carbon atoms to which they are attached to form a 5- or 6-membered substituted or unsubstituted carbocyclic ring; or C₁-C₁₀ haloalkyl, C₁-C₁₀ alkoxy, C₁—. C₁₀ haloalkoxy, C₄-C₈ cycloalkoxy, phenoxy, halo, hydroxy, carboxyl, —SH, —CN, —S(C₁-C₁₀ alkyl), arylthio, thioacetal, —C(O)O(C₁-C₁₀ alkyl), hydrazino, hydrazido, —NH₂, —NO₂, —NR₈₂R₈₃, and —C(O)NR₈₂R₈₃, where, R₈₂ and R₈₃ are independently hydrogen, C₁-C₁₀ alkyl, C₁-C₁₀ hydroxyalkyl, or taken together with N, R₈₂ and R₈₃ form a 5- to 8-membered heterocyclic ring; or a group having the formula;

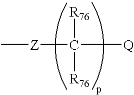

where,

each R₇₆ is independently selected from Hydrogen, C₁-C₁₀ alkyl, hydroxy, or both R₇₆ taken together are ═O;

P is 1 to 8,

Z is a bond, —O—, —N(C₁-C₁₀ alkyl)-, —NH, or —S—; and

Q is —CON(R₈₂R₈₃), -5-tetrazolyl, —SO₃H,

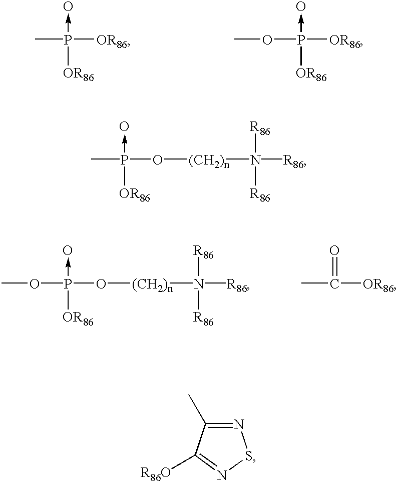

where R₈₆ is independently selected from hydrogen, a metal, or C₁-C₁₀ alkyl.

C) 1H-indole-3-acetamide sPLA₂ inhibitors and methods of making these inhibitors are set out in U.S. Pat. No. 5,684,034, the entire disclosure of which is incorporated herein by reference. The method of the invention is for treatment of a mammal, including a human, afflicted with renal dysfunction, said method comprising administering to said human a therapeutically effective amount of the compound represented by (IIb), and pharmaceutically acceptable salts and prodrug derivatives thereof,

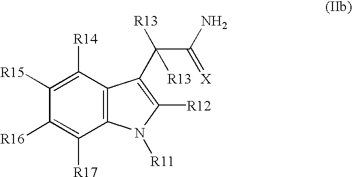

wherein

X is oxygen or sulfur;

R₁₁ is selected from groups (i), (ii) (iii) and (iv) where;

(i) is C₆-C₂₀ alkyl, C₆-C₂₀ alkenyl, C₆-C₂₀ alkynyl, C₆-C₂₀ haloalkyl, C₄-C₁₂ cycloalkyl, or

(ii) is aryl or aryl substituted by halo, nitro, —CN, —CHO, —OH, —SH, C₁-C₁₀ alkyl, C₁-C₁₀ alkylthio, C₁-C₁₀ alkoxyl, carboxyl, amino, or hydroxyamino; or

(iii) is —(CH₂)ₙ—(R₈₀), or —(NH)—(R₈₁), where n is 1 to 8, and R₈₀ is a group recited in (i), and R₈₁ is selected from a group recited in (i) or (ii);

(iv) is

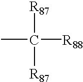

where R₈₇ is hydrogen or C₁-C₁₀ alkyl, and R₈₈ is selected from the group; phenyl, naphthyl, indenyl, and biphenyl, unsubstituted or substituted by halo, —CN, —CHO, —OH, —SH, C₁-C₁₀ alkylthio, C₁-C₁₀ alkoxyl, phenyl, nitro, C₁-C₁₀ alkyl, C₁-C₁₀ haloalkyl, carboxyl, amino, hydroxyamino; or a substituted or unsubstituted 5 to 8 membered heterocyclic ring;

R₁₂ is halo, C₁-C₂ alkylthio, or C₁-C₂ alkoxy;

each R₁₃ is independently hydrogen, halo, or methyl;

R₁₄, R₁₅, R₁₆, and R₁₇ are each independently hydrogen, C₁-C₁₀ alkyl, C₁-C₁₀ alkenyl, C₁-C₁₀ alkynyl, C₃-C₈ cycloalkyl, aryl, aralkyl, or any two adjacent hydrocarbyl groups in the set R₁₄, R₁₅, R₁₆, and R₁₇, combine with the ring carbon atoms to which they are attached to form a 5 or 6 membered substituted or unsubstituted carbocyclic ring; or C₁-C₁₀ haloalkyl, C₁-C₁₀ alkoxy, C₁-C₁₀ haloalkoxy, C₄-C₈ cycloalkoxy, phenoxy, halo, hydroxy, carboxyl, —SH, —CN, C₁-C₁₀ alkylthio, arylthio, thioacetal, —C(O)O(C₁-C₁₀ alkyl), hydrazide, hydrazino, hydrazido, —NH₂, —NO₂, —NR₈₂R₈₃, and —C(O)NR₈₂R₈₃, where, R₈₂ and R₈₃ are independently hydrogen, C₁-C₁₀ alkyl, C₁-C₁₀ hydroxyalkyl, or taken together with N, R₈₂ and R₈₃ form a 5- to 8-membered heterocyclic ring; or a group having the formula;

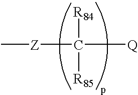

where,

R₈₄ and R₈₅ are each independently selected from Hydrogen, C₁-C₁₀ alkyl, hydroxy, or R₈₄ and R₈₅ taken together are ═O;

P is 1 to 5,

Z is a bond, —O—, —N(C₁-C₁₀ alkyl)-, —NH—, or —S—; and

Q is —CON(R₈₂R₈₃), -5-tetrazolyl, —SO₃H,

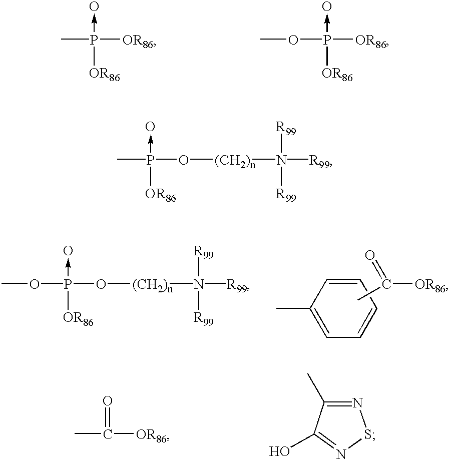

where n is 1 to 8, R₈₆ is independently selected from hydrogen, a metal, or C₁-C₁₀ alkyl, and R₉₉ is selected from hydrogen or C₁-C₁₀ alkyl.

D) 1H-indole-1-functional sPLA₂ inhibitors of the hydrazide, amide, or glyoxylamide types as described in U.S. Pat. No. 5,641,800, the entire disclosure of which is incorporated herein by reference are useful for treatment of a mammal, including a human afflicted with renal dysfunction. 1H-indole-1-acetamide or a pharmaceutically acceptable salt, solvate or prodrug derivative thereof; wherein said compound is represented by the formula (Ic);

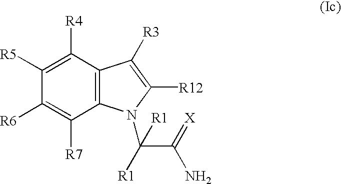

wherein for Formula (Ic);

X is oxygen or sulfur;

each R¹ is independently hydrogen, or C₁-C₃ alkyl;

R₃ is selected from groups (a), (b) and (c) where;

(a) is C₇-C₂₀ alkyl, C₇-C₂₀ alkenyl, C₇-C₂₀ alkynyl, carbocyclic radical, or heterocyclic radical, or

(b) is a member of (a) substituted with one or more independently selected non-interfering substituents; or

(c) is the group -(L)-R₈₀; where, -(L)- is a divalent linking group of 1 to 12 atoms

and where R₈₀ is a group selected from (a) or (b);

R₂ is hydrogen, halo, C₁-C₃ alkyl, C₃-C₄ cycloalkyl, C₃-C₄ cycloalkenyl, —O—(C₁-C₂ alkyl), —S—(C₁-C₂ alkyl), or a non-interfering substituent having a total of 1 to 3 atoms other than hydrogen;

R₆ and R₇ are independently selected from hydrogen, a non-interfering substituent, or the group, -(Lₐ)-(acidic group); wherein -(Lₐ)-, is an acid linker having an acid linker length of 1 to 10; provided, that at least one of R₆ and R₇ must be the group, -(Lₐ)-(acidic group);

R₄ and R₅ are each independently selected from hydrogen, non-interfering substituent, carbocyclic radical, carbocyclic radical substituted with non-interfering substituents, heterocyclic radical, and heterocyclic radical substituted with non-interfering substituents.

1H-indole-1-hydrazide compounds useful as sPLA₂ inhibitors in the practice of the method of the invention are as follows:

1H-indole-1-hydrazide compound or a pharmaceutically acceptable salt, solvate or prodrug derivative thereof; is represented by the formula (IIc);

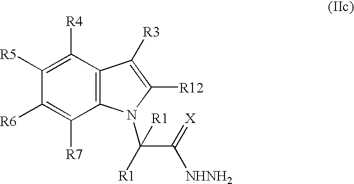

wherein for formula (IIc);

X is oxygen or sulfur;

Each R₁ is independently hydrogen, or C₁-C₃ alkyl;

R₃ is selected from groups (a), (b) and (c) where;

(a) is C₇-C₂₀ alkyl, C₇-C₂₀ alkenyl, C₇-C₂₀ alkynyl, carbocyclic radical, or heterocyclic radical, or

(b) is a member of (a) substituted with one or more independently selected non-interfering substituent; or

(c) is the group -(L)-R₈₀; where, -(L)- is a divalent linking group of 1 to 12 atoms and where R₈₀ is a group selected from (a) or (b);

R₂ is hydrogen, halo, C₁-C₃ alkyl, C₃-C₄ cycloalkyl, C₃-C₄ cycloalkenyl, —O—(C₁-C₂ alkyl), —S—(C₁-C₂ alkyl), or a non-interfering substituent having a total of 1 † to 3 atoms other than hydrogen;

R₆ and R₇ are independently selected from hydrogen, a non-interfering substituent, or the group, -(Lₐ)-(acidic group); wherein -(Lₐ)-, is an acid linker having an acid linker length of 1 to 10; provided, that at least one of R₆ and R₇ must be the group, -(Lₐ)-(acidic group);

R₄ and R₅ are each independently selected from hydrogen, non-interfering substituent, carbocyclic radical, carbocyclic radical substituted with non-interfering substituents, heterocyclic radical, and heterocyclic radical substituted with non-interfering substituents.

E) Indolizine sPLA₂ inhibitors and their method of preparation are described in U.S. patent application Ser. No. 08/765,566, filed Jul. 20, 1995 (titled, “Synovial Phospholipase A2 Inhibitor Compounds Having an Indolizine Type Nucleus, Parmaceutical Formulations Containing Said compounds, and Therapeutic Methods of Using said Compounds”), the entire disclosure of which is incorporated herein by reference; and also in European Patent Publication No. 0772596, published May 14, 1997. The 1H-indole-1-functional compound or a pharmaceutically acceptable salt, solvate or prodrug derivative thereof; is represented by the formula (Id);

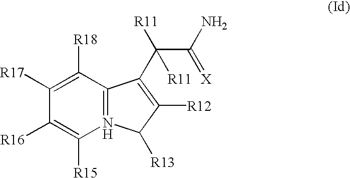

wherein;

X is oxygen or sulfur;

each R¹¹ is independently hydrogen, C₁-C₃ alkyl, or halo;

R₁₃ is selected from groups (a), (b) and (c) where;

(a) is C₇-C₂₀ alkyl, C₇-C₂₀ alkenyl, C₇-C₂₀ alkynyl, carbocyclic radical, or heterocyclic radical, or

(b) is a member of (a) substituted with one or more independently selected non-interfering substituents; or

(c) is the group -(L)-R₈₀; where, -(L)- is a divalent linking group of 1 to 12 atoms and where R₈₀ is a group selected from (a) or (b);

R₁₂ is hydrogen, halo, C₁-C₃ alkyl, C₃-C₄ cycloalkyl, C₃-C₄ cycloalkenyl, —O—(C₁-C₂ alkyl), —S—(C₁-C₂ alkyl), or a non-interfering substituent having a total of 1 to 3 atoms other than hydrogen;

R₁₇ and R₁₈ are independently selected from hydrogen, a non-interfering substituent, or the group, -(Lₐ)-(acidic group); wherein -(Lₐ)-, is an acid linker having an acid linker length of 1 to 10; provided, that at least one of R₁₇ and R₁₈ must be the group, -(Lₐ)-(acidic group); and

R₁₅ and R₁₆ are each independently selected from hydrogen, non-interfering substituent, carbocyclic radical, carbocyclic radical substituted with non-interfering substituents, heterocyclic radical, and heterocyclic radical substituted with non-interfering substituents.

Particularly preferred 1H-indole-1-functional compounds useful as sPLA₂ inhibitors in the practice of the method of the invention are as follows:

an indolizine-1-acetic acid hydrazide compound or a pharmaceutically acceptable salt, solvate or prodrug derivative thereof where said compound is represented by the formula (IId);

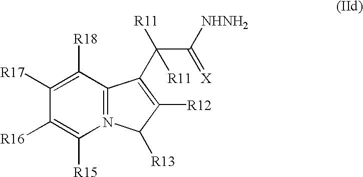

particularly preferred 1H-indole-1-functional compounds useful as sPLA₂ inhibitors in the practice of the method of the invention are as follows:

an indolizine-1-glyoxylamide compound or a pharmaceutically acceptable salt, solvate or prodrug derivative thereof; wherein said compound is represented by the formula (IIId);

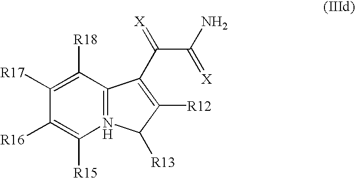

another preferred 1H-indole-1-functional compounds useful as sPLA₂ inhibitors in the practice of the method of the invention are as follows:

an indolizine-3-acetamide compound or a pharmaceutically acceptable salt, solvate or prodrug derivative thereof; wherein said compound is represented by the formula (IVd), as set out below:

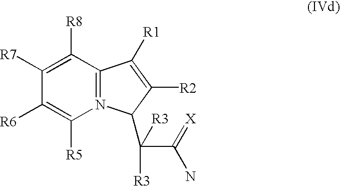

wherein;

X is selected from oxygen or sulfur;

each R₃ is independently hydrogen, C₁-C₃ alkyl, or halo;

R¹ is selected from groups (a), (b) and (c) where;

(a) is C₇-C₂₀ alkyl, C₇-C₂₀ alkenyl, C₇-C₂₀ alkynyl, carbocyclic radical, or heterocyclic radical, or

(b) is a member of (a) substituted with one or more independently selected non-interfering substituents; or

(c) is the group -(L)-R₈₀; where, -(L)- is a divalent linking group of 1 to 12 atoms and where R₈₀ is a group selected from (a) or (b);

R₂ is hydrogen, halo, C₁-C₃ alkyl, C₃-C₄ cycloalkyl, C₃-C₄ cycloalkenyl, —O—(C₁-C₂ alkyl), —S—(C₁-C₂ alkyl), or a non-interfering substituent having a total of 1 to 3 atoms other than hydrogen;

R₅ and R₆ are independently selected from hydrogen, a non-interfering substituent, or the group, -(Lₐ)-(acidic group); wherein -(Lₐ)-, is an acid linker having an acid linker length of 1 to 10; provided, that at least one of R₅ and R₆ must be the group, -(Lₐ)-(acidic group);

R₇ and R₈ are each independently selected from hydrogen, non-interfering substituent, carbocyclic radical, carbocyclic radical substituted with non-interfering substituents, heterocyclic radical, and heterocyclic radical substituted with non-interfering substituents.

Particularly preferred 1H-indole-1-functional compounds useful as sPLA₂ inhibitors in the practice of the method of the invention are as follows:

an indolizine-3-hydrazide compound or a pharmaceutically acceptable, salt, solvate or prodrug derivative thereof; wherein said compound is represented by the formula (Vd), as set out below:

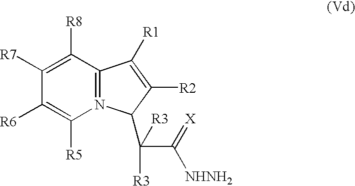

Particularly preferred 1H-indole-1-functional compounds useful as sPLA₂ inhibitors in the practice of the method of the invention are as follows:

an indolizine-3-glyoxylamide compound or a pharmaceutically acceptable salt, solvate or prodrug derivative thereof; wherein said compound is represented by the formula (vid), as set out below:

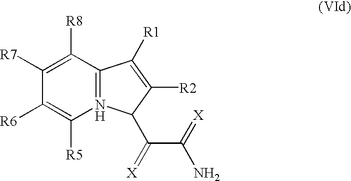

particularly preferred 1H-indole-1-functional compounds useful as sPLA₂ inhibitors in the practice of the method of the invention are as follows:

an indolizine-1-acetamide functional compound or a pharmaceutically acceptable salt, solvate or prodrug derivative thereof; wherein said compound is selected from the group represented by the following formulae:

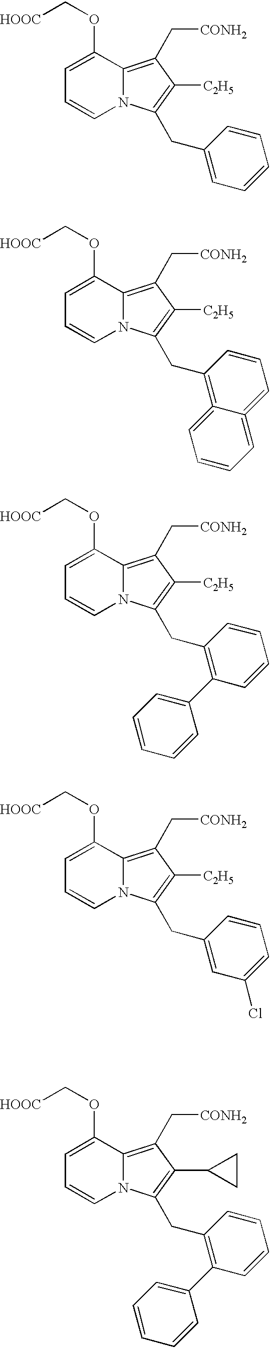

and mixtures of the above compounds.

F) Indene sPLA₂ inhibitors as described in U.S. patent application Ser. No. 08/776,618 filed Jul. 20, 1995, (titled, Synovial Phospholipase A2 Inhibitor Compounds having an Indene Type Nucleus, Pharmaceutical Formulations Containing said Compounds, and Therapeutic Methods of Using Said Compounds”), the entire disclosure of which is incorporated herein by reference, are useful in practicing the method of the invention.

An indene-1-acetamide compound or a pharmaceutically acceptable salt, solvate or prodrug derivative thereof; is is represented by the formula (If);

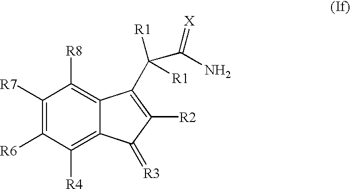

wherein;

X is oxygen or sulfur;

each R¹ is independently hydrogen, C₁-C₃ alkyl, or halo;

R₃ is selected from groups (a), (b) and (c) where;

(a) is C₇-C₂₀ alkyl, C₇-C₂₀ alkenyl, C₇-C₂₀ alkynyl, carbocyclic radical, or heterocyclic radical, or

(b) is a member of (a) substituted with one or more independently selected non-interfering substituents; or

(c) is the group -(L)-R₈₀; where, -(L)- is a divalent linking group of 1 to 12 atoms and where R₈₀ is a group selected from (a) or (b);

R₂ is hydrogen, halo, C₁-C₃ alkyl, C₃-C₄ cycloalkyl, C₃-C₄ cycloalkenyl, —O—(C₁-C₂ alkyl), —S—(C₁-C₂ alkyl), or a non-interfering substituent having a total of 1 to 3 atoms other than hydrogen;

R₆ and R₇ are independently selected from hydrogen, a non-interfering substituent, or the group, -(Lₐ)-(acidic group); wherein -(Lₐ)-, is an acid linker having an acid linker length of 1 to 10; provided, that at least one of R₆ and R₇ must be the group, -(Lₐ)-(acidic group); and R₄ and R₅ are each independently selected from hydrogen, non-interfering substituent, carbocyclic radical, carbocyclic radical substituted with non-interfering substituents, heterocyclic radical, and heterocyclic radical substituted with non-interfering substituents.

Suitable indene compounds also include the following:

an indene-1-acetic acid hydrazide compound or a pharmaceutically acceptable salt, solvate or prodrug derivative thereof; wherein said compound is represented by the formula (iif);

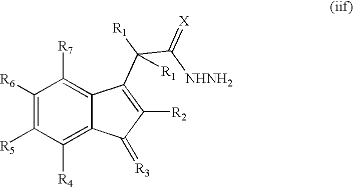

wherein:

X is oxygen or sulfur;

each R₁ is independently hydrogen, C₁-C₃ alkyl, or halo;

R₃ is selected from groups (a), (b) and (c) where;

(a) is C₇-C₂₀ alkyl, C₇-C₂₀ alkenyl, C₇-C₂₀ alkynyl, carbocyclic radical, or heterocyclic radical, or

(b) is a member of (a) substituted with one or more independently selected non-interfering substituents; or

(c) is the group -(L)-R₈₀; where, -(L)- is a divalent linking group of 1 to 12 atoms and where R₈₀ is a group selected from (a) or (b);

R₂ is hydrogen, halo, C₁-C₃ alkyl, C₃-C₄ cycloalkyl, C₃-C₄ cycloalkenyl, —O—(C₁-C₂ alkyl), —S—(C₁-C₂ alkyl), or a non-interfering substituent having a total of 1 to 3 atoms other than hydrogen;

R₆ and R₇ are independently selected from hydrogen, a non-interfering substituent, or the group, -(Lₐ)-(acidic group); wherein -(Lₐ)-, is an acid linker having an acid linker length of 1 to 10; provided, that at least one of R₆ and R₇ must be the group, -(Lₐ)-(acidic group); and

R₄ and R₅ are each independently selected from hydrogen, non-interfering substituent, carbocyclic radical, carbocyclic radical substituted with non-interfering substituents, heterocyclic radical, and heterocyclic radical substituted with non-interfering substituents.

Suitable indene compounds for use in the method of the invention also include the following:

an indene-1-glyoxylamide compound or a pharmaceutically acceptable salt, solvate or prodrug derivative thereof; wherein said compound is represented by the formula (iiif);

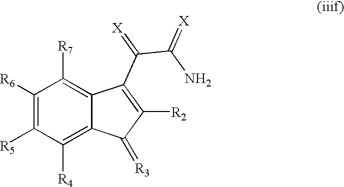

X is oxygen or sulfur;

R₃ is selected from groups (a), (b) and (c) where;

(a) is C₇-C₂₀ alkyl, C₇-C₂₀ alkenyl, C₇-C₂₀ alkynyl, carbocyclic radical, or heterocyclic radical, or

(b) is a member of (a) substituted with one or more independently selected non-interfering substituents; or

(c) is the group -(L)-R₈₀; where, -(L)- is a divalent linking group of 1 to 12 atoms and where R₈₀ is a group selected from (a) or (b);

R₂ is hydrogen, halo, C₁-C₃ alkyl, C₃-C₄ cycloalkyl, C₃-C₄ cycloalkenyl, —O—(C₁-C₂ alkyl), —S—(C₁-C₂ alkyl), or a non-interfering substituent having a total of 1 to 3 atoms other than hydrogen;

R₆ and R₇ are independently selected from hydrogen, a non-interfering substituent, or the group, -(Lₐ)—(acidic group); wherein -(Lₐ)-, is an acid linker having an acid linker length of 1 to 1a; provided, that at least one of R₆ and R₇ must be the group, -(Lₐ)-(acidic group);

R₄ and R₅ are each independently selected from hydrogen, non-interfering substituent, carbocyclic radical, carbocyclic radical substituted with non-interfering substituents, heterocyclic radical, and heterocyclic radical substituted with non-interfering substituents.

G) Carbazole and tetrahydrocarbazole sPLA₂ inhibitors and methods of making these compounds are set out in U.S. patent application Ser. No. 09/063,066 filed Apr. 21, 1998 (titled, “Substituted Carbazoles and 1,2,3,4-Tetrahydrocarbazoles”), the entire disclosure of which is incorporated herein by reference. The method of the invention includes treatment of a mammal with these compounds.

The method of the invention is for treatment of a mammal, including a human, afflicted with renal dysfunction, said method comprising administering to said human a therapeutically effective amount carbazole or tetrahydrocarbazole represented by the following:

a compound of the formula (Ie)

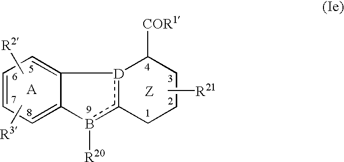

wherein;

a is phenyl or pyridyl wherein the nitrogen is at the 5-, 6-, 7- or 8-position;

one of B or D is nitrogen and the other is carbon; Z is cyclohexenyl, phenyl, pyridyl, wherein the nitrogen is at the 1-, 2-, or 3-position, or a 6-membered heterocyclic ring having one heteroatom selected from the group consisting of sulfur or oxygen at the 1-, 2- or 3-position, and nitrogen at the 1-, 2-, 3- or 4-position;

[symbol]

is a double or single bond;

R²⁰ is selected from groups (a), (b) and (c) where;

(a) is —(C₅-C₂₀)alkyl, —(C₅-C₂₀)alkenyl, —(C₅-C₂₀), alkynyl, carbocyclic radicals, or heterocyclic radicals, or

(b) is a member of (a) substituted with one or more independently selected non-interfering substituents; or

(c) is the group -(L)-R⁸⁰; where, -(L)- is a divalent linking group of 1 to 12 atoms selected from carbon, hydrogen, oxygen, nitrogen, and sulfur; wherein the combination of atoms in -(L)- are selected from the group consisting of (i) carbon and hydrogen only, (ii) one sulfur only, (iii) one oxygen only, (iv) one or two nitrogen and hydrogen only, (v) carbon, hydrogen, and one sulfur only, and (vi) and carbon, hydrogen, and oxygen only; and where R⁸⁰ is a group selected from (a) or (b);

R²¹ is a non-interfering substituent;

R₁′ is —NHNH₂, —NH₂ or —CONH₂;

R₂′ is selected from the group consisting of —OH, and —O(CH₂)ₜr5′ where

R^{5′} is H, —CN, —NH₂, —CONH₂, —CONR₉R¹⁰—NHSO₂R¹⁵; —CONHSO₂R¹⁵, where R¹⁵ is —(C₁-C₆)alkyl or —CF₃; phenyl or phenyl substituted with —CO₂H or —CO₂(C₁-C₄)alkyl; and -(Lₐ)-(acidic group), wherein -(Lₐ)- is an acid linker having an acid linker length of 1 to 7 and t is 1-5;

R³, is selected from non-interfering substituent, carbocyclic radicals, carbocyclic radicals substituted with non-interfering substituents, heterocyclic radicals, and heterocyclic radicals substituted with non-interfering substituents; or a pharmaceutically acceptable racemate, solvate, tautomer, optical isomer, prodrug derivative or salt thereof;

provided that; when R³, is H, R²⁰ is benzyl and m is 1 or 2; R^{2′} cannot be —O(CH₂)ₘh; and

Provided that when D is nitrogen, the heteroatom of Z is selected from the group consisting of sulfur or oxygen at the l-, 2- or 3-position and nitrogen at the l-, 2-, 3- or 4-position.

Preferred in the practice of the method of the invention are compounds represented by the formula (IIe):

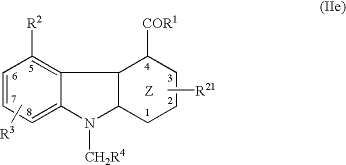

wherein;

Z is cyclohexenyl, or phenyl;

R²¹ is a non-interfering substituent;

R¹ is —NHHH₂ or —NH₂;

R² is selected from the group consisting of —OH and —O(CH₂)ₘ^{r5} where

R⁵ is H, —CO₂H, —CONH₂, —CO₂(C₁-C₄ alkyl);

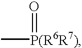

where R⁶ and R⁷ are each independently —OH or —O(C₁-C₄)alkyl; —SO₃H, —SO₃(C₁-C₄ alkyl), tetrazolyl, —CN, —NH₂, —NHSO₂R¹⁵; —CONHSO₂R¹⁵, where R¹⁵ is —(C₁-C₆)alkyl or —CF₃, phenyl or phenyl substituted with —CO₂H or —CO₂(C₁-C₄)alkyl where m is 1-3;

R³ is H, —O(C₁-C₄)alkyl, halo, —(C₁-C₆)alkyl, phenyl, —(C₁-C₄)alkylphenyl; phenyl substituted with —(C₁-C₆)alkyl, halo, or —CF₃; —CH₂OSi(C₁-C₆)alkyl, furyl, thiophenyl, —(C₁-C₆)hydroxyalkyl; or —(CH₂)ₙr⁸ where R⁸ is H, —CONH₂, —NR⁹R¹⁰, —CN or phenyl where R⁹ and R¹⁰ are independently —(C₁-C₄)alkyl or -phenyl(C₁-C₄)alkyl and n is 1 to 8;

R⁴ is H, —(C₅-C₁₄)alkyl, —(C₃-C₁₄)cycloalkyl, pyridyl, phenyl or phenyl substituted with —(C₁-C₆)alkyl, halo, —CF₃, —OCF₃, —(C₁-C₄)alkoxy, —CN, —(C₁-C₄)alkylthio, phenyl(C₁-C₄)alkyl, —(C₁-C₄)alkylphenyl, phenyl, phenoxy or naphthyl; or a pharmaceutically acceptable racemate, solvate, tautomer, optical isomer, prodrug derivative or salt, thereof.

Preferred specific compounds including all salts and prodrug derivatives thereof, for practicing the method of the invention are as follows:

9-benzyl-5,7-dimethoxy-1,2,3,4-tetrahydrocarbazole-4-carboxylic acid hydrazide;

9-benzyl-5,7-dimethoxy-1,2,3,4-tetrahydrocarbazole-4-carboxamide;

[9-benzyl-4-carbamoyl-7-methoxy-1,2,3,4-tetrahydrocarbazol-5-yl]oxyacetic acid sodium salt;

[9-benzyl-4-carbamoyl-7-methoxycarbazol-5-yl]oxyacetic acid;

Methyl [9-benzyl-4-carbamoyl-7-methoxycarbazol-5-yl]oxyacetic acid;

9-benzyl-7-methoxy-5-cyanomethyloxy-1,2,3,4-tetrahydrocarbazole-4-carboxamide;

9-benzyl-7-methoxy-5-(1H-tetrazol-5-yl-methyl)oxy)-1,2,3,4-tetrahydrocarbazole-4-carboxamide;

{9-[(phenyl)methyl]-5-carbamoyl-2-methyl-carbazol-4-yl}oxyacetic acid;

{9-[(3-fluorophenyl)methyl]-5-carbamoyl-2-methyl-carbazol-4-yl}oxyacetic acid;

{9-[(3-methylphenyl)methyl]-5-carbamoyl-2-methyl-carbazol-4-yl}oxyacetic acid;

{9-[(phenyl)methyl]-5-carbamoyl-2-(4-trifluoromethylphenyl)-carbazol-4-yl}oxyacetic acid;

9-benzyl-5-(2-methanesulfonamido)ethyloxy-7-methoxy-1,2,3,4-tetrahydrocarbazole-4-carboxamide;

9-benzyl-4-(2-methanesulfonamido)ethyloxy-2-methoxycarbazole-5-carboxamide;

9-benzyl-4-(2-trifluoromethanesulfonamido)ethyloxy-2-methoxycarbazole-5-carboxamide;

9-benzyl-5-methanesulfonamidoylmethyloxy-7-methoxy-1,2,3,4-tetrahydrocarbazole-4-carboxamide;

9-benzyl-4-methanesulfonamidoylmethyloxy-carbazole-5-carboxamide;

[5-carbamoyl-2-pentyl-9-(phenylmethyl)carbazol-4-yl]oxyacetic acid;

[5-carbamoyl-2-(1-methylethyl)-9-(phenylmethyl)carbazol-4-yl]oxyacetic acid;

[5-carbamoyl-9-(phenylmethyl)-2-[(tri (-1-methylethyl)silyl)oxymethyl]carbazol-4-yl]oxyacetic acid;

[5-carbamoyl-2-phenyl-9-(phenylmethyl)carbazol-4-yl]oxyacetic acid[5-carbamoyl-2-(4-chlorophenyl)-9-(phenylmethyl)carbazol-4-yl]oxyacetic acid;

[5-carbamoyl-2-(2-furyl)-9-(phenylmethyl) carbazol-4-yl]oxyacetic acid;

[5-carbamoyl-9-(phenylmethyl)-2-[(tri(-1-methylethyl)silyl)oxymethyl]carbazol-4-yl]oxyacetic acid, lithium salt;

{9-[(phenyl)methyl]-5-carbamoylcarbazol-4-yl}oxyacetic acid;

{9-[(3-fluorophenyl)methyl]-5-carbamoylcarbazol-4-yl}oxyacetic acid;

{9-[(3-phenoxyphenyl)methyl]-5-carbamoylcarbazol-4-yl}oxyacetic acid;

{9-[(2-Fluorophenyl)methyl]-5-carbamoylcarbazol-4-yl}oxyacetic acid;

{9-[(2-trifluoromethylphenyl)methyl]-5-carbamoylcarbazol-4-yl}oxyacetic acid;

{9-[(2-benzylphenyl)methyl]-5-carbamoylcarbazol-4-yl}oxyacetic acid;

{9-[(3-trifluoromethylphenylYmethyl]-5-carbamoylcarbazol-4-yl}oxyacetic acid;

{9-[(1-naphthyl)methyl]-5-carbamoylcarbazol-4-yl}oxyacetic acid;

{9-[(2-cyanophenyl)methyl]-5-carbamoylcarbazol-4-yl}oxyacetic acid;

{9-[(3-cyanophenyl)methyl]-5-carbamoylcarbazol-4-yl}oxyacetic acid;

{9-[(2-methylphenyl)methyl]-5-carbamoylcarbazol-4-yl}oxyacetic acid;

{9-[(3-methylphenyl)methyl]-5-carbamoylcarbazol-4-yl}oxyacetic acid;

{9-[(3,5-dimethylphenyl)methyl]-5-carbamoylcarbazol-4-yl}oxyacetic acid;

{9-[(3-iodophenyl)methyl]-5-carbamoylcarbazol-4-yl}oxyacetic acid;

{9-[(2-Chlorophenyl)methyl]-5-carbamoylcarbazol-4-yl}oxyacetic acid;

{9-[(2,3-difluorophenyl)methyl]-5-carbamoylcarbazol-4-yl}oxyacetic acid;

{9-[(2,6-difluorophenyl)methyl]-5-carbamoylcarbazol-4-yl}oxyacetic acid;

{9-[(2,6-dichlorophenyl)methyl]-5-carbamoylcarbazol-4-yl}oxyacetic acid;

{9-[(3-trifluoromethoxyphenyl)methyl]-5-carbamoylcarbazol-4-yl}oxyacetic acid;

{9-[(2-biphenyl)methyl]-5-carbamoylcarbazol-4-yl}oxyacetic acid;

{9-[(2-Biphenyl)methyl)-5-carbamoylcarbazol-4-yl}oxyacetic acid; the {9-[(2-Biphenyl)methyl]-5-carbamoylcarbazol-4-yl}oxyacetic acid;

[9-Benzyl-4-carbamoyl-1,2,3,4-tetrahydrocarbaole-5-yl]oxyacetic acid;

{9-[(2-Pyridyl)methyl]-5-carbamoylcarbazol-4-yl}oxyacetic acid;

{9-[(3-Pyridyl)methyl]-5-carbamoylcarbazol-4-yl}oxyacetic acid;

[9-benzyl-4-carbamoyl-8-methyl-1,2,3,4-tetrahydrocarbazol-5-yl]oxyacetic acid;

[9-benzyl-5-carbamoyl-1-methylcarbazol-4-yl]oxyacetic acid;

[9-benzyl-4-carbamoyl-8-fluoro-1,2,3,4-tetrahydrocarbazol-5-yl]oxyacetic acid;

[9-benzyl-5-carbamoyl-1-fluorocarbazol-4-yl]oxyacetic acid;

[9-benzyl-4-carbamoyl-8-chloro-1,2,3,4-tetrahydrocarbazol-5-yl]oxyacetic acid;

[9-benzyl-5-carbamoyl-1-chlorocarbazol-4-yl]oxyacetic acid;

[9-[(Cyclohexyl)methyl]-5-carbamoylcarbazol-4-yl]oxyacetic acid;

[9-[(Cyclopentyl)methyl]-5-carbamoylcarbazol-4-yl]oxyacetic acid;

5-carbamoyl-9-(phenylmethyl)-2-[[(propen-3-yl)oxy]methyl]carbazol-4-yl]oxyacetic acid;

[5-carbamoyl-9-(phenylmethyl)-2-[(propyloxy)methyl]carbazol-4-yl]oxyacetic acid;

9-benzyl-7-methoxy-5-((carboxamidomethyl)oxy)-1,2,3,4-tetrahydrocarbazole-4-carboxamide;

9-benzyl-7-methoxy-5-cyanomethyloxy-carbazole-4-carboxamide;

9-benzyl-7-methoxy-5-((1H-tetrazol-5-yl-methyl)oxy)-carbazole-4-carboxamide;

9-benzyl-7-methoxy-5-((carboxamidomethyl)oxy)-carbazole-4-carboxamide; and

[9-Benzyl-4-carbamoyl-1,2,3,4-tetrahydrocarbaole-5-yl]oxyacetic acid or a pharmaceutically acceptable racemate, solvate, tautomer, optical isomer, prodrug derivative or salt, thereof.

Other desirable carbazole compounds suitable for practicing the method of thein invention are selected from those represented by the formula (XXX):

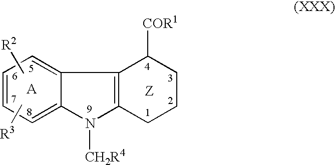

wherein:

R¹ is —NHNH₂, or —NH_{2;}

R² is selected from the group consisting of —OH and —O(CH₂)ₘr⁵ where

R⁵ is H, —CO₂H, —CO₂(C1-C4 alkyl);

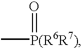

where R⁶ and R⁷ are each independently —OH or —O(C₁-C₄)alkyl; —SO₃H, —SO₃(C₁-C₄ alkyl), tetrazolyl, —CN, —NH₂, —NHSO₂R¹⁵; —CONHSO₂R¹⁵, where R¹⁵ is —(C₁-C₆)alkyl or —CF₃, phenyl or phenyl substituted with —CO₂H or —CO₂(C₁-C₄)alkyl where m is 1-3;

R³ is H, —O(C₁-C₄)alkyl, halo, —(C₁-C₆)alkyl, phenyl, —(C₁-C₄)alkylphenyl; phenyl substituted with —(C₁-C₆)alkyl, halo, or —CF₃; —CH₂OSi(C₁-C₆)alkyl, furyl, thiophenyl, —(C₁-C₆)hydroxyalkyl; or —(CH₂)ₙr⁸ where R⁸ is H, —CONH₂, —NR⁹R¹⁰, —CN or phenyl where R⁹ and R¹⁰ are independently —(C₁-C₄)alkyl or -phenyl(C₁-C₄)alkyl and n is 1 to 8;

R⁴ is H, —(C₅-C₁₄)alkyl, —(C₃-C₁₄)cycloalkyl, pyridyl, phenyl or phenyl substituted with —(C₁-C₆)alkyl, halo, —CF₃, —OCF₃, —(C₁-C₄)alkoxy, —CN, —(C₁-C₄)alkylthio, phenyl(C₁-C₄)alkyl, —(C₁-C₄)alkylphenyl, phenyl, phenoxy or naphthyl;

a is phenyl or pyridyl wherein the nitrogen is at the 5-, 6-, 7- or 8-position;

Z is cyclohexenyl, phenyl, pyridyl wherein the nitrogen is at the 1-, 2- or 3-position or a 6-membered heterocyclic ring having one heteroatom selected from the group consisting of sulfur or oxygen at the 1-, 2- or 3-position and nitrogen at the 1-, 2-, 3- or 4-position, or

wherein one carbon on the heterocyclic ring is optionally substituted with ═O; or a pharmaceutically acceptable racemate, solvate, tautomer, optical isomer, prodrug derivative or salt thereof;

provided that one of A or Z is a heterocyclic ring.

Further desirable specific compounds suitable for the method of the invention are selected from the following:

(R, S)-(9-benzyl-4-carbamoyl-1-oxo-3-thia-1,2,3,4-tetrahydrocarbazol-5-yl)oxyacetic acid; (R,S)-(9-benzyl-4-carbamoyl-1-oxo-3-thia-1,2,3,4-tetrahydrocarbazol-5-yl)oxyacetic acid; [N-benzyl-1-carbamoyl-1-aza-1,2,3,4-tetrahydrocarbazol-8-yl]oxyacetic acid; 4-methoxy-6-methoxycarbonyl-10-phenylmethyl-6,7,8,9-tetrahydropyrido[1,2-a]indole; (4-carboxamido-9-phenylmethyl-4,5-dihydrothiopyrano[3,4-b]indol-5-yl)oxyacetic acid; 3,4-dihydro-4-carboxamidol-5-methoxy-9-phenylmethylpyrano[3,4-b]indole; 2-[(2,9 bis-benzyl-4-carbamoyl-1,2,3,4-tetrahydro-beta-carbolin-5-yl)oxy]acetic acid or a pharmaceutically acceptable racemate, solvate, tautomer, optical isomer, prodrug derivative or salt thereof.

Particularly preferred compounds for the treatment of renal dysfunction are represented by the formulae (Xe) and (xie) below:

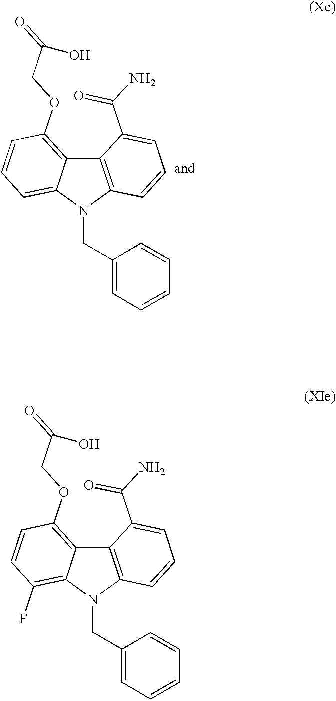

For all of the above compounds of the carbazole or tetrahydrocarbazole type it is advantageous to use them in their (i) acid form, or (ii) pharmaceutically acceptable (e.g., Na, K) form, or (iii) and prodrugs derivatives (e.g., Methyl ester, ethyl ester, n-butyl ester, morpholino ethyl ester).

Prodrugs are derivatives of sPLA₂ inhibitors used in the method of the invention which have chemically or metabolically cleavable groups and become by solvolysis or under physiological conditions the compounds of the invention which are pharmaceutically active in vivo. Derivatives of the compounds of this invention have activity in both their acid and base derivative forms, but the acid derivative form often offers advantages of solubility, tissue compatibility, or delayed release in a mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acidic compound with a suitable alcohol, or amides prepared by reaction of the parent acid compound with a suitable amine. Simple aliphatic or aromatic esters derived from acidic groups pendent on the compounds of this invention are preferred prodrugs. In some cases it is desirable to prepare double ester type prodrugs such as (acyloxy) alkyl esters or ((alkoxycarbonyl)oxy)alkyl esters. Specific preferred prodrugs are ester prodrugs inclusive of methyl ester, ethyl ester, n-propyl ester, isopropyl ester, n-butyl ester, sec-butyl, tert-butyl ester, N,N-diethylglycolamido ester, and morpholino-N-ethyl ester. Methods of making ester prodrugs are disclosed in U.S. Pat. No. 5,654,326. Additional methods of prodrug synthesis are disclosed in U.S. Provisional Patent Application Serial No. 60/063,280 filed Oct. 27, 1997 (titled, N,N-diethylglycolamido ester Prodrugs of Indole sPLA₂ Inhibitors), the entire disclosure of which is incorporated herein by reference; U.S. Provisional Patent Application Serial No. 60/063,646 filed Oct. 27, 1997 (titled, Morpholino-N-ethyl Ester Prodrugs of Indole sPLA₂ Inhibitors), the entire disclosure of which is incorporated herein by reference; and U.S. Provisional Patent Application Serial No. 60/063,284 filed Oct. 27, 1997 (titled, Isopropyl Ester Prodrugs of Indole sPLA₂ Inhibitors), the entire disclosure of which is incorporated herein by reference.

Carbazole and tetrahydrocarbazole sPLA₂ inhibitor compounds useful for practicing the method of the invention may be made by the following general methods:

the compounds of formula Ie where Z is cyclohexene are prepared according to the following reaction Schemes Ig (a) and (c).

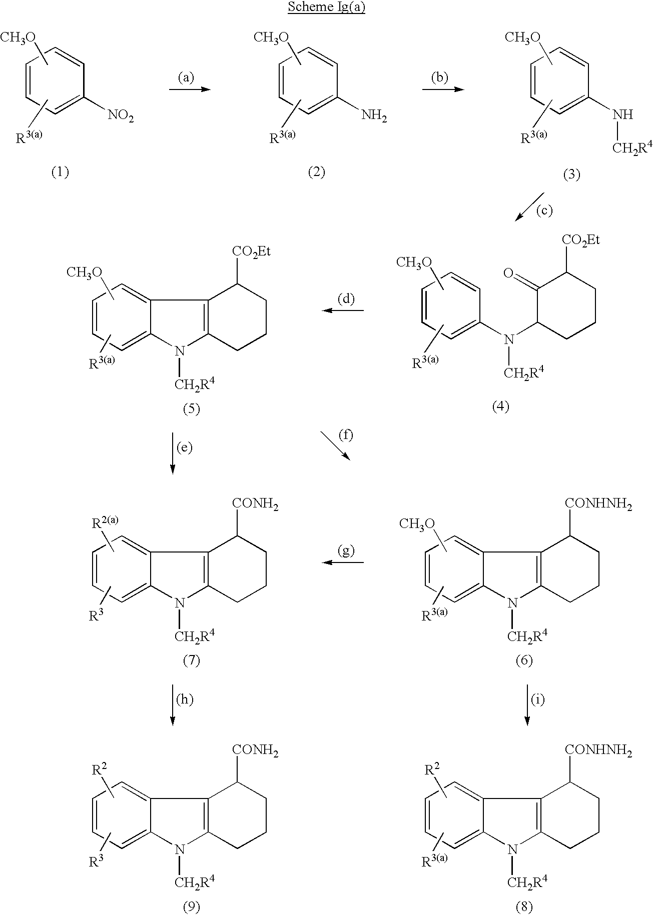

wherein;

R¹ is —NH², R³(a) is H, —O(C₁-C₄)alkyl, halo, —(C₁-C₆)alkyl, phenyl, —(C₁-C₄)alkylphenyl; phenyl substituted with —(C₁-C₆)alkyl, halo, or —CF₃; —CH₂OSi(C₁-C₆)alkyl, furyl, thiophenyl, —(C₁-C₆)hydroxyalkyl, —(C₁-C₆)alkoxy(C₁-C₆)alkyl, —(C₁-C₆)alkoxy(C₁-C₆)alkenyl; or —(CH₂)ₙr⁸ where R⁸ is H, —CONH₂, —NR⁹R¹⁰, —CN or phenyl where R⁹ and R¹⁰ are independently hydrogen, —CF₃, phenyl, —(C₁-C₄)alkyl, —(C₁-C₄)alkylphenyl or -phenyl(C₁-C₄)alkyl and n is 1 to 8;

when R¹ is —NHNH², R³(a) is H, —O(C₁-C₄)alkyl, halo, —(C₁-C₆)alkyl, phenyl, —(C₁-C₄)alkylphenyl; phenyl substituted with —(C₁-C₆)alkyl, halo or —CF₃; —CH₂OSi(C₁-C₆)alkyl, furyl, thiophenyl, —(C₁-C₆)hydroxyalkyl, —(C₁-C₆)alkoxy(C₁-C₆)alkyl, —(C₁-C₆)alkoxy(C₁-C₆)alkenyl; or —(CH₂)ₙr⁸ where R⁸ is H, —NR⁹R¹⁰, —CN or phenyl where R⁹ and R¹⁰ are independently hydrogen, —CF₃, phenyl, —(C₁-C₄)alkyl, —(C₁-C₄)alkylphenyl or -phenyl(C₁-C₄)alkyl and n is 1 to 8;

R^{2(a)} is —OCH₃ or —OH.

An appropriately substituted nitrobenzene (1) can be reduced to the aniline (2) by treatment with a reducing agent, such as hydrogen in the presence of Pd/C, preferably at room temperature.

Compound (2) is N-alkylated at temperatures of from about 0 to 20° C. using an alkylating agent such as an appropriately substituted aldehyde and sodium cyanoborohydride to form (3). Alternately, an appropriately substituted benzyl halide may be used for the first alkylation step. The resulting intermediate is further N-alkylated by treatment with 2-carbethoxy-6-bromocyclohexanone, preferably at temperatures of about 80° C. to yield (4) or by treatment with potassium hexamethyldisilazide and the bromoketoester.

The product (4) is cyclized to the tetrahydrocarbazole (5) by refluxing with zncl₂ in benzene for from about 1 to 2 days, preferably at 80 C (see Julia, M.; Lenzi, J. Preparation d′acides tetrahydro-1,2,3,4-carbazole-1 ou -4. Bull. Soc. Chim. France, 1962, 2262-2263). Compound (5) is converted to the hydrazide (6) by treatment with hydrazine at temperatures of about 100° C., or to the amide (7) by reacting with methylchloroaluminum amide in benzene (see Levin, J. I.; Turos, E.; Weinreb, S. M. An alternative procedure for the aluminum-mediated conversion of esters to amides. Syn. Comm., 1982, 12, 989-993). Alternatively, (7) may be produced by treatment of (6) with Raney nickel active catalyst.

It will be readily appreciated that when R³(a) is:

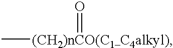

Conversion to the amide will also be achieved in this procedure.

Compounds (6) and (7) may be dealkylated, preferably at 0° C. to room temperature, with a dealkylating agent, such as boron tribromide or sodium thioethoxide, to give compound (7) where R²(a) is —OH, which may then be further converted to compound (9), by realkylating with a base, such as sodium hydride, and an alkylating agent, such as Br(CH₂)ₘr⁵, where R⁵ is the carboxylate or phosphonic diester or nitrile as defined above. Conversion of R² to the carboxylic acid may be accomplished by treatment with an aqueous base. When R² is nitrile, conversion to the tetrazole may be achieved by reacting with tri-butyl tin azide or conversion to the carboxamide may be achieved by reacting with basic hydrogen peroxide. When R² is the phosphonic diester, conversion to the acid may be achieved by reacting with a dealkylating agent such as trimethylsilyl bromide. The monoester may be accomplished by reacting the diester with an aqueous base.

When R² and R³ are both methoxy, selective demethylation can be achieved by treating with sodium ethanethiolate in dimethylformamide at 100° C.

An alternative synthesis of intermediate (5) is shown in Scheme I(b), as follows.

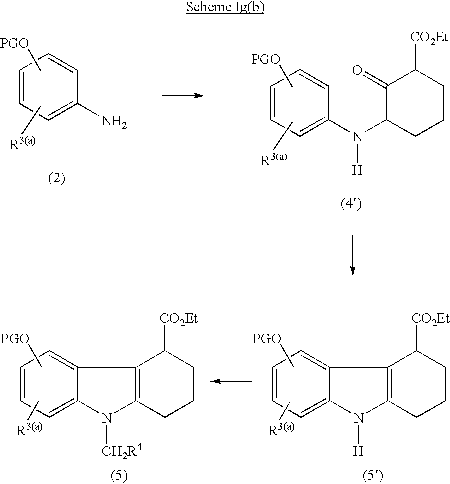

where PG is a protecting group;

R³a is as defined in Scheme 1, above.

The aniline (2) is N-alkylated with 2-carbethoxy-6-bromocyclohexanone in dimethyl formamide in the presence of sodium bicarbonate for 8-24 hours at 50° C. Preferred protecting groups include methyl, carbonate, and silyl groups, such as t-butyldimethylsilyl. The reaction product (4′) is cyclized to (5′) using the zncl₂ in benzene conditions described in Scheme I(a), above. N-alkylation of (5′) to yield (5) is accomplished by treatment with sodium hydride and the appropriate alkyl halide in dimethylformamide at room temperature for 4-8 hours.

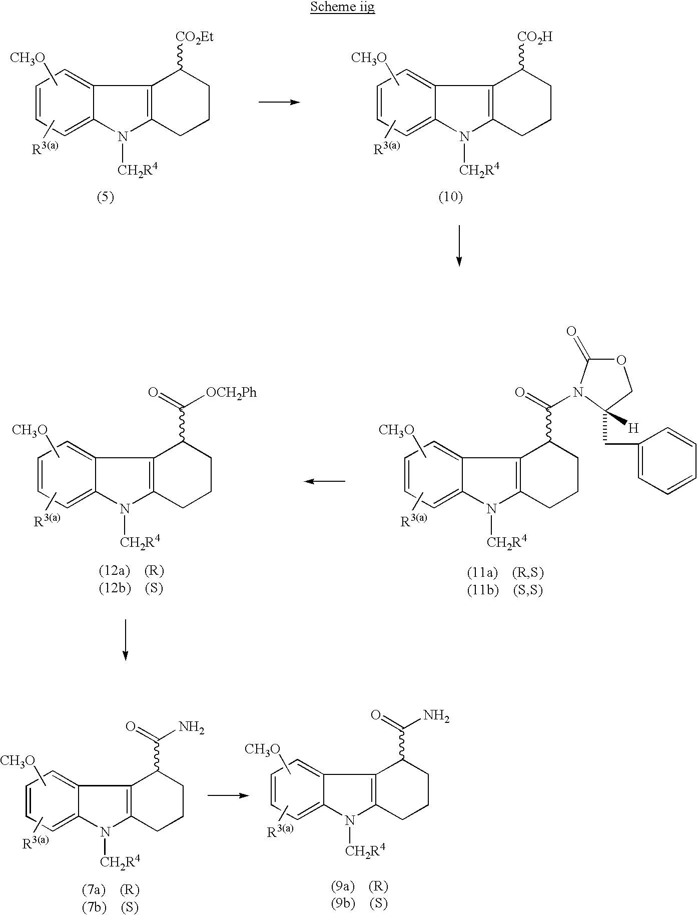

R³(a) is as defined in Scheme Ig.

As discussed in Scheme I above, carbazole (5) is hydrolyzed to the carboxylic acid (10) by treatment with an aqueous base, preferably at room temperature to about 100° C. The intermediate is then converted to an acid chloride utilizing, for example, oxalyl chloride and dimethylformamide, and then further reacted with a lithium salt of (S) or (R)-4-alkyl-2-oxazolidine at a temperature of about −75° C., to give (11a) and (11b), which are separable by chromatography.

The diastereomers are converted to the corresponding enantiomeric benzyl esters (12) by brief treatment at temperatures of about 0° C. to room temperature with lithium benzyl oxide (see Evans, D. A.; Ennis, M. D.; Mathre, D. J. Asymmetric alkylation reactions of chiral imide enolates. A practical approach to the enantioselective synthesis of alpha-substituted carboxylic acid derivatives J. Am. Chem. Soc., 1982, 104, 1737-1738).

The esters (12) are then converted to (7) preferably by treatment with methylchloroaluminum amide (Ref 2, above) or, alternately, by hydrogenation using, for example, hydrogen and palladium on carbon, as described above, to make the acid and then reacting with an acyl azide, such as diphenylphosphoryl azide followed by treatment with ammonia. Using the procedure described above in Scheme I, compound (9a) or (9b) may be accomplished.

Compounds of formula Ie where Z is phenyl can be prepared as follows in Schemes III(a)-(f), below.

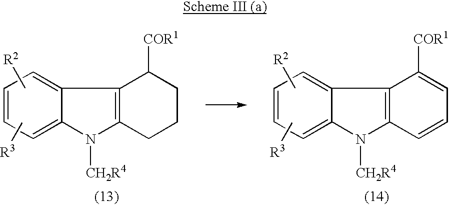

A 1,2,3,4-tetrahydrocarbazole-4-carboxamide or 4-carboxhydrazide (13) is dehydrogenated by refluxing in a solvent such as carbitol in the presence of Pd/C to produce the carbazole-4-carboxamide. Alternately, treatment of (13) with DDQ in an appropriate solvent such as dioxane yields carbozole (14).

Depending on the substituent pattern oxidation as described above may result in de-alkylation of the nitrogen. For example when R³ is substituted at the 8-position with methyl, oxidation results in dealkylation of the nitrogen which may be realkylated by treatment with sodium hydride and the appropriate alkyl halide as described in Scheme I(a) above to prepare the deired product (14).

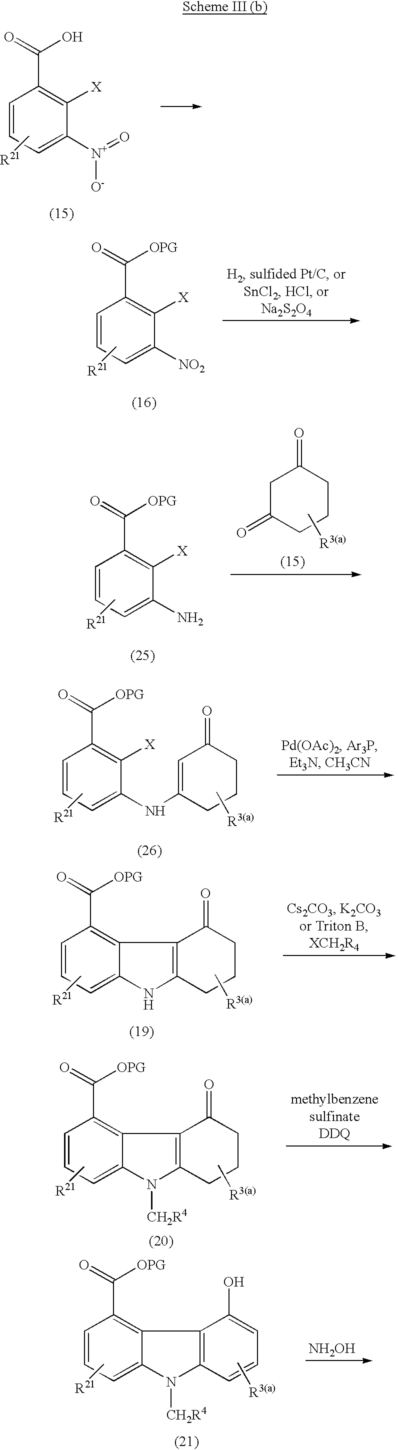

Benzoic acid derivative(16) where X is preferably chlorine, bromine or iodine and the protecting group is preferably —CH₃, are reduced to the corresponding aniline (25) with a reducing agent, such as stannous chloride in the presence of acid under the general conditions of Sakamoto et al, Chem Pharm. Bull. 35 (5), 1823-1828 (1987).

Alternatively, reduction with sodium dithionite in the presence of a base, such as sodium carbonate in a noninterferring solvent, such as water, ethanol, and/or tetrahydrofuran affords starting material (16).

Alternatively, reduction by hydrogenation over a sulfided platinum catalyst supported on carbon with hydrogen at 1 to 60 atmospheres in a noninterfering solvent, preferably ethyl acetate, to form a starting material (16).

The reactions are conducted at temperatures from about 0 to 100° C. Preferably at ambient temperature, and are substantially complete in about 1 to 48 hours depending on conditions.

The aniline (25) and dione (15) are condensed under dehydrating conditions, for example, using the general procedure of Iida, et al., (Ref 5), with or without a noninterfering solvent, such as toluene, benzene, or methylene chloride, under dehydrating conditions at a temperature about 10 to 150° C. The water formed in the process can be removed by distillation, azetropic removal via a Dean-Stark apparatus, or the addition of a drying agent, such as molecular sieves, magnesium sulfate, calcium carbonate, sodium sulfate, and the like.

The process can be performed with or without a catalytic amount of an acid, such a p-toluenesulfonic acid or methanesulfonic acid. Other examples of suitable catalysts include hydrochloric acid, phenylsulfonic acid, calcium chloride, and acetic acid.

Examples of other suitable solvents include tetrahydrofuran, ethyl acetate, methanol, ethanol, 1, 1,2,2-tetrachloroethane, chlorobenzene, bromobenzene, xylenes, and carbotetrachloride.

The condensation of the instant process is preferably carried out neat, at a temperature about 100 to 150° C. with the resultant water removed by distillation via a stream of inert gas, such as, nitrogen or argon.

The reaction is substantially complete in about 30 minutes to 24 hours.

Intermediate (26) may then be readily cyclized in the presence of a palladium catalyst, such as Pd(oac)₂ or Pd(pph₃)₄ and the like, a phosphine, preferably a trialkyl- or triarylphosphine, such as triphenylphosphine, tri-o-tolylphosphine, or tricyclohexylphosphine, and the like, a base, such as, sodium bicarbonate, triethylamine, or diisopropylethylamine, in a noninterfering solvent, such as, acetonitrile, triethylamine, or toluene at a temperature about 25 to 200° C. to form (19).

Examples of other suitable solvents include tetrahydrofuran, benzene, dimethylsulfoxide, or dimethylformamide.

Examples of other suitable palladium catalysts include Pd(pph₃)Cl₂, Pd(OCOCF₃)₂, [(CH₃C₆H₄)₃P]2pdcl₂, [(CH₃CH₂)₃P]₂pdcl₂, [(C₆H11)₃P]₂pdcl₂, and [(C₆H₅)₃P]2pdbr₂.

Examples of other suitable phosphines include triisopropylphosphine, triethylphosphine, tricyclopentylphosphine, 1,2-bis(diphenylphosphino)ethane, 1,3-bis(diphenylphosphino)propane, and 1,4-bis(diphenylphosphino)butane.

Examples of other suitable bases include tripropyl amine, 2,2,6,6-tetramethylpiperidine, 1,5-diazabicyclo[2.2.2]octane (DABCO), 1,8-diazabicyclo[5.4.0]undec-7-ene (DBU), 1,5-diazabicyclo[4.3.0]non-5-ene, (DBN) sodium carbonate, potassium carbonate, and potassium bicarbonate.

The cyclization of the instant process is preferably carried out with palladium(II)acetate as catalyst in the presence of either triphenylphosphine, tri-o-tolylphosphine, 1,3-bis(diphenylphosphino)propane, or tricyclohexylphosphine in acetonitrile as solvent and triethylamine as base at a temperature about 50 to 150° C. The reaction is substantially complete in about 1 hour to 14 days.

Alternatively, a preferred process for cyclization consists of the reaction of intermediate (26) with a palladacycle catalyst such as trans-di(μ-acetato)-bis[o-(di-o-tolylphosphino)benzyl]dipalladium (II) in a solvent such as dimethylacetamide (DMAC) at 120-140° C. in the presence of a base such as sodium acetate.

Intermediate (19) may be alkylated with an alkylating agent XCH₂R₄, where X is halo in the presence of a base to form (20). Suitable bases include potassium carbonate, sodium carbonate, lithium carbonate, cesium carbonate, sodium bicarbonate, potassium bicarbonate, potassium hydroxide, sodium hydroxide, sodium hydride, potassium hydride, lithium hydride, and Triton B (N-benzyltrimethylammonium hydroxide).

The reaction may or may not be carried out in the presence of a crown ether. Potassium carbonate and Triton B are preferred. The amount of alkylating agent is not critical, however, the reaction is best accomplished using an excess of alkyl halide relative to the starting material.

A catalytic amount of an iodide, such as sodium iodide or lithium iodide may or may not be added to the reaction mixture. The reaction is preferably carried out in an organic solvent, such as, acetone, dimethylformamide, dimethylsulfoxide, or acetonitrile. Other suitable solvents include tetrahydrofuran, methyl ethyl ketone, and t-butyl methyl ether.

The reaction is conducted at temperatures from about −10 to 100° C. Preferably at ambient temperature, and is substantially complete in about 1 to 48 hours depending on conditions. Optionally, a phase transfer reagent such as tetrabutylammonium bromide or tetrabutylammonium chloride may be employed.

Intermediate (20) May by dehydrogenated by oxidation with 2,3-dichloro-5,6-dicyano-1,4-benzoquinone in a noninterfering solvent to form (21).

Suitable solvents include methylene chloride, chloroform, carbon tetrachloride, diethyl ether, methyl ethyl ketone, and t-butyl methyl ether. Toluene, benzene, dioxane, and tetrahydrofuran are preferred solvents. The reaction is carried out at a temperature about 0 to 120° C. Temperatures from 50 to 120° C. are preferred. The reaction is substantially complete in about 1 to 48 hours depending on conditions.

Intermediate (21) may be aminated with ammonia in the presence of a noninterfering solvent to form a(22). Ammonia may be in the form of ammonia gas or an ammonium salt, such as ammonium hydroxide, ammonium acetate, ammonium trifluoroacetate, anmonium chloride, and the like. Suitable solvents include ethanol, methanol, propanol, butanol, tetrahydrofuran, dioxane, and water. A mixture of concentrated aqueous ammonium hydroxide and tetrahydrofuran or methanol is preferred for the instant process. The reaction is carried out at a temperature about 20 to 100° C. Temperatures from 50 to 60° C. are preferred. The reaction is substantially complete in about 1 to 48 hours depending on conditions.

Alkylation of (22) is achieved by treatment with an alkylating agent of the formula XCH₂R⁹ where X is halo and R⁷⁰ is —CO₂R⁷¹, —SO₃R⁷¹, —P(O)(OR⁷¹)₂, or —P(O)(OR⁷¹)H, where R⁷¹ is an acid protecting group or a prodrug function, in the presence of a base in a noninterfering solvent to form (23). Methyl bromoacetate and t-butyl bromoacetate are the preferred alkylating agents.

Suitable bases include potassium carbonate, sodium carbonate, lithium carbonate, cesium carbonate, sodium bicarbonate, potassium bicarbonate, potassium hydroxide, sodium hydroxide, sodium hydride, potassium hydride, lithium hydride, and Triton B (N-benzyltrimethylammonium hydroxide). The reaction may or may not be carried out in the presence of a crown ether. Cesium carbonate and Triton B are preferred.

The amount of alkylating agent is not critical, however, the reaction is best accomplished using an excess of alkyl halide relative to the starting material. The reaction is preferably carried out in an organic solvent, such as, acetone, dimethylformamide, dimethylsulfoxide, or acetonitrile. Other suitable solvents include tetrahydrofuran, methyl ethyl ketone, and t-butyl methyl ether.

The reaction is conducted at temperatures from about −10 to 100° C. Preferably at ambient temperature, and is substantially complete in about 1 to 48 hours depending on conditions. Optionally, a phase transfer reagent such as tetrabutylammonium bromide or tetrabutylammonium chloride may be employed.

Intermediate (23) may be optionally hydrolyzed with a base or acid to form desired product (24) and optionally salified.

Hydrolysis of (23) is achieved using a base such as sodium hydroxide, potassium hydroxide, lithium hydroxide, aqueous potassium carbonate, aqueous sodium carbonate, aqueous lithium carbonate, aqueous potassium bicarbonate, aqueous sodium bicarbonate, aqueous lithium bicarbonate, preferably sodium hydroxide and a lower alcohol solvent, such as, methanol, ethanol, isopropanol, and the like. Other suitable solvents include acetone, tetrahydrofuran, and dioxane.

Alternatively, the acid protecting group may be removed by organic and inorganic acids, such as trifluoroacetic acid and hydrochloric acid with or without a noninterferring solvent. Suitable solvents include methylene chloride, tetrahydrofuran, dioxane, and acetone. The t-butyl esters are preferably removed by neat trifluoroacetic acid.

The reaction is conducted at temperatures from about −10 to 100° C. Preferably at ambient temperature, and is substantially complete in about 1 to 48 hours depending on conditions.

The starting material (16) is prepared by esterifying compound (15) with a alkyl halide=XPG; where X is halo and PG is an acid protecting group, in the presence of a base, preferably potassium carbonate or sodium cabonate, in a noninterferring solvent, preferably dimethylformamide or dimethylsulfoxide. The preferred alkyl halide is methyl iodide. The reaction is conducted at temperatures from about 0 to 100° C. Preferably at ambient temperature, and is substantially complete in about 1 to 48 hours depending on conditions.

Alternatively the starting material (16) may be prepared by condensation with an alcohol HOPG, where PG is an acid protecting group, in the presence of a dehydrating catalyst such as, dicyclohexylcarbodiimide (DCC) or carbonyl diimidazole.

In addition, U.S. Pat. No. 4,885,338 and Jpn. Kokai Tokkyo Koho 05286912, November 1993 Hesei teach a method for preparing 2-fluoro-5-methoxyaniline derivatives.

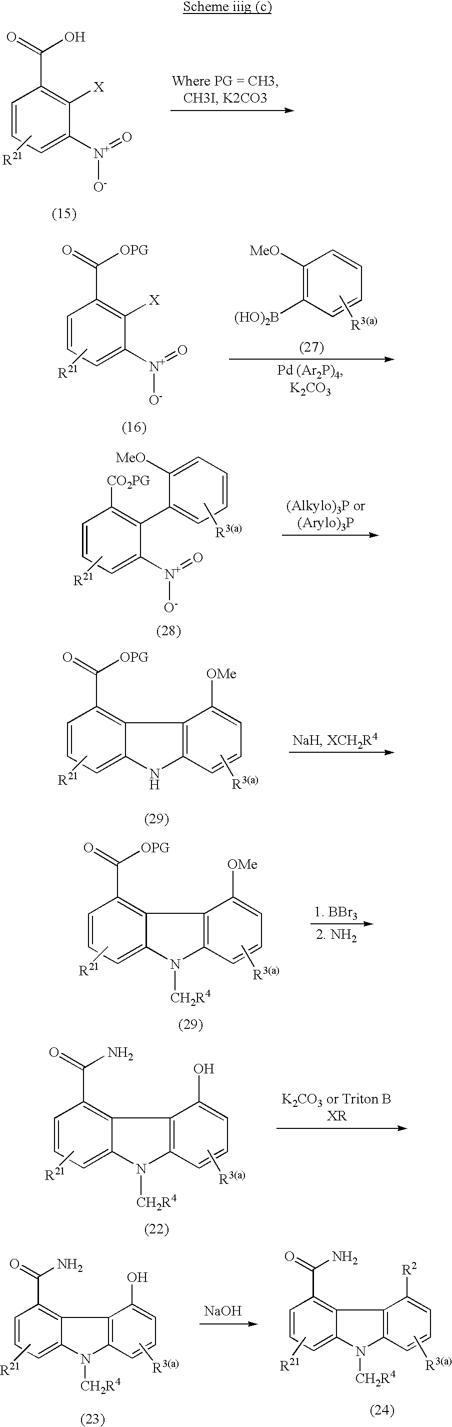

R is as defined in Scheme iiig(b),

R^{3(a)} is as defined in Scheme Ig(a), above; and

X is halo.

Benzoic acid derivatives (16) (X═Cl, Br, or I) and boronic acid derivative (27) (either commercially available or readily prepared by known techniques from commercially available starting materials) are condensed under the general procedure of Miyaura, et al., (Ref 8a) or Trecourt, et al., (Ref 8b) in the presence of a palladium catalyst, such as Pd(Ph₃P)₄, a base, such as sodium bicarbonate, in an inert solvent, such as THF, toluene or ethanol, to afford compound (28).

Compound (28) is converted to the carbazole product (29) by treatment with a trialkyl or triaryl phosphite or phosphine, such as, triethylphosphite or triphenyl phosphine, according to the general procedure of Cadogan, et al., J. Chem. Soc., 4831 (1965).

Compound (29) is N-alkylated with an appropriately substituted alkyl or aryl halide XCH₂R⁴ in the presence of a base, such as sodium hydride or potassium carbonate, in a noninterfering solvent, such as toluene, dimethylformamide, or dimethylsulfoxide to afford carbazole (30).

Compound (30) is converted to the corresponding amide (22) by treatment with boron tribromide or sodium thioethoxide, followed by ammonia or an ammonium salt, such as ammonium acetate, in an inert solvent, such as water or alcohol, or with methylchloroaluminum amide in an inert solvent, such as toluene, at a temperature between 0 to 110° C.

When R^{3(a)} is substituted at the 8-position with chloro, de-alkylation of (30) with boron tribromide results in de-benzylation of the nitrogen as described above. Alkylation may be readily accomplished in a two step process. First, an O-alkylation by treatment with a haloalkyl acetate such as methyl bromo acetate using sodium hydride in tetrahydrofuran, followed by N-alkylation using for example a base such as sodium hydride and an appropriately substituted alkyl or aryl halide in dimethoxy formamide. Compound (22) can be converted to product carbazole product (24) as described previously in Scheme iiig(b) above.

Conversion to the desired prodrug may be accomplished by techniques known to the skilled artisan, such as for example, by treatment with a primary or secondary halide to make an ester prodrug.

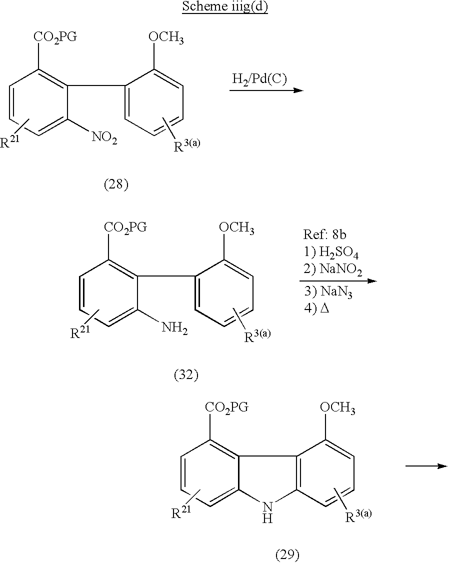

Alternatively, reduction of the nitro group of compound (28) with a reducing agent, such as hydrogen in the presence of palladium on carbon, in a noninterfering solvent, such as ethanol, at 1 to 60 atmospheres, at a temperature of 0 to 60° C. affords the corresponding aniline (32). Compound (32) is converted to the carbazole (29>according to the general procedure described by Miyaura, et al., Synthetic Communications 11, 523(1981). The aniline is treated with sulfuric acid and sodium nitrite, followed by sodium azide to form an intermediate azide which is cyclized to carbazole (29) by heating in an inert sovent, such as toluene. Compound (29) is converted to carbazole product (24) as described previously in Schemes iiig(b) and iiig(c).

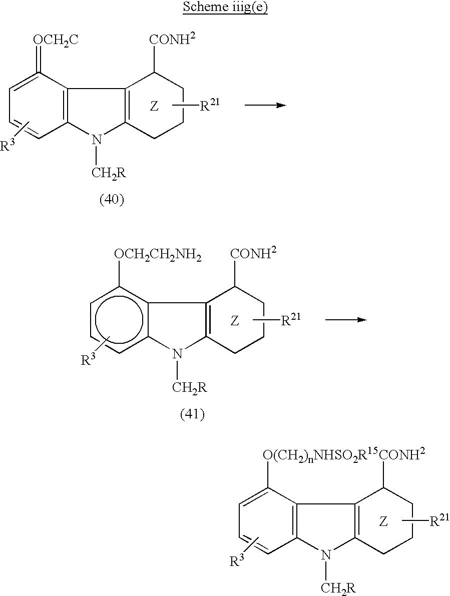

In an aprotic solvent, preferably tetrahydrofuran, reduction of (40) is achieved using a reducing agent such as aluminum trihydride. Preferably, the reaction is conducted under inert atmosphere such as nitrogen, at room temperature. Sulfonylation may be achieved with an appropriate acylating agent in the presence of an acid scavenger such as triethyl amine.

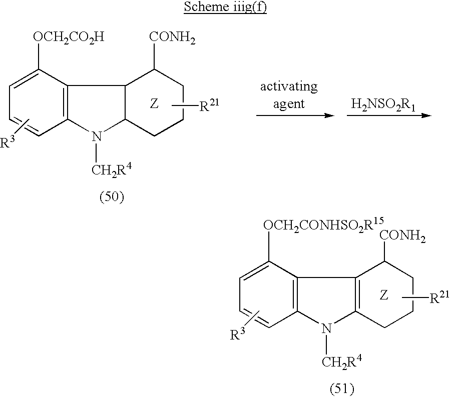

In a two-step, one-pot process, intermediate (50), prepared as described in Scheme I(a) above, is first activated with an activating agent such as carbonyl diimidazole. The reaction is preferably run in an aprotic polar or non-polar solvent such as tetrahydrofuran. Acylation with the activated intermediate is accomplished by reacting with H₂NSOR¹⁵ in the presence of a base, preferably diazabicycloundecene.

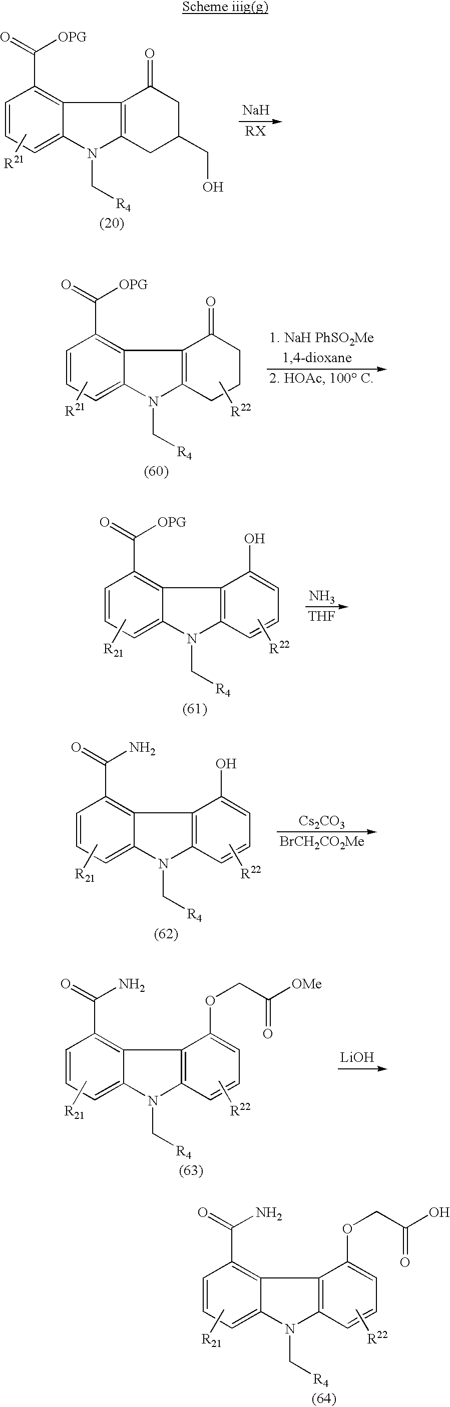

PG is an acid protecting group;

R²² is (C₁-C₆)alkoxy (C₁-C₆)alkyl is (C₁-C₆)alkoxy (C₁-C₆)alkenyl.

Starting material (20) is O-alkylated with an alkyl halide or alkenyl halide, using a base such as NaH, in an aprotic polar solvent preferably anhydrous DMF, at ambient temperature under a nitrogen atmosphere. The process of aromatization from a cyclohexenone functionality to a phenol functionality can be performed by treating the tetrahydrocabazole intermediate (60) with a base such as NaH in the presence of methyl benzenesulfinate in an anhydrous solvent, such as 1,4-dioxane or DMF, to form the ketosulfoxide derivative. Upon heating at about 100° C. for 1-2 hours, the ketosulfoxide derivative (60) is converted to the phenol derivative (61). Conversion of the ester (61) to the amide (62) can be achieved by treating a solution of (61) in an aprotic polar solvent such as tetrahydrofuran with ammonia gas. Phenolic O-alkylation of (62) with, for example, methyl bromoacetate can be carried out in anhydrous DMF at ambient temperature using Cs₂CO₃ or K₂CO₃ as a base to form (63). Desired product (64) can be derived from the basic hydrolysis of ester (63) using LiOH or NaOH as a base in an H₂O/CH₃OH/THF solution at 50° C. for 1-2 hours.

When R²² is —(C₁-C₆)alkoxy(C₁-C₆)alkenyl, hydrogenation of the double bond can be performed by treating (63) in THF using PtO₂ as a catalysis under a hydrogen atmosphere. Desired product can then be derived as described above in Scheme III(g) from the basic hydrolysis of ester (63) using LiOH or NaOH as a base in an H₂O/CH₃OH/THF solution at 50° C. for 1-2 hours.

H) Pyrazole sPLA₂ Inhibitors

The method of the invention may be practiced using pyrazole sPLA₂ inhibitors, which are described (together with the method of making) in US Patent Application No. 08/984,261, filed Dec. 3, 1997, the entire disclosure of which is incorporated herein by reference. Suitable pyrazole compounds are represented by formula (Ih)

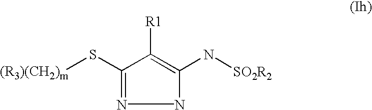

wherein:

R¹ is phenyl, isoquinolin-3-yl, pyrazinyl, pyridin-2-yl, pyridin-2-yl substituted at the 4-position with —(C₁-C₄)alkyl, (C₁-C₄)alkoxyl, —CN or —(CH₂)ₙconh₂ where n is 0-2;

R² is phenyl; phenyl substituted with 1 to 3 substituents selected from the group consisting of —(C₁-C₄)alkyl, —CN, halo, —NO₂, CO₂(C₁-C₄)alkyl and —CF₃; naphthyl; thiophene or thiophene substituted with 1 to 3 halo groups;

R³ is hydrogen; phenyl; phenyl(C₂-C₆)alkenyl; pyridyl; naphthyl; quinolinyl; (C₁-C₄)alkylthiazolyl;

Phenyl substituted with 1 to 2 substituents selected from the group consisting of —(C₁-C₄)alkyl, —CN, —CONH₂, —NO₂, —CF₃, halo, (C₁-C₄)alkoxy, CO₂(C₁-C₄)alkyl, phenoxy and SR⁴ where R⁴ is —(C₁-C₄)alkyl or halophenyl;

Phenyl substituted with one substituent selected from the group consisting of

—O(CH₂)ₚr⁵ where p is 1 to 3 and R⁵ is —CN, —CO₂H, —CONH₂, or tetrazolyl,

Phenyl and

—OR⁶ where R⁶ is cyclopentyl, cyclohexenyl, or phenyl substituted with halo or (C₁-C₄)alkoxy;

Or phenyl substituted with two substituents which, when taken together with the phenyl ring to which they are attached form a ethylenedioxy ring; and M is 1 to 5; or a pharmaceutically acceptable salt thereof.

Particularly preferred are pyrazole type sPLA₂ inhibitors as follows:

a pyrazole compound of formula (I), supra, wherein:

R¹ is pyridine-2-yl or pyridine-2-yl substituted at the 4-position with —(C₁-C₄)alkyl, (C₁-C₄)alkoxy, —CN or —(CH₂)ₙCONH₂ where n is 0-2;

R² is phenyl substituted with 1 to 3 substituents selected from the group consisting of —(C₁-C₄)alkyl, —CN, halo, —NO₂, CO₂(C₁-C₄)alkyl and —CF₃; and

R³ is phenyl; phenyl(C₂-C₆)alkenyl; phenyl substituted with 1 or 2 substituents selected from the group consisting of —(C₁-C₄)alkyl, —CN, —CONH₂, —NO₂, —CF₃, halo, (C₁-C₄)alkoxy, CO₂(C₁-C₄)alkyl, phenoxy and SR₄ where R⁴ is —(C₁-C₄)alkyl or halo phenyl;

phenyl substituted with one substituent selected from the group consisting of —O(CH₂)ₚr⁵ where p is 1 to 3 and R⁵ is —CN, —CO₂H, —CONH₂ or tetrazolyl, phenyl and —OR⁶ where R⁶ is cyclopentyl, cyclohexenyl or phenyl substituted with halo or (C₁-C₄)alkoxy;

or phenyl substituted with two substituents which when taken together with the phenyl ring to which they are attached form a methylenedioxy ring.

Specific suitable pyrazole type sPLA₂ inhibitors useful in the method of the invention are as follows: Compounds selected from the group consisting of 3-(2-chloro-6-methylphenylsulfonylamino)-4-(2-(4-acetamido)pyridyl)-5-(3-(4-fluorophenoxy)benzylthio)-(1H)-pyrazole and 3-(2,6-dichlorophenylsulfonylamino)-4-(2-(4-acetamido)pyridyl)-5-(3-(4-fluorophenoxy)benzylthio)-(1H)-pyrazole.

I) Phenyl glyoxamide sPLA₂ inhibitors (and the method of making them) are described in U.S. patent application Ser. No. 08/979,446, filed Nov. 24, 1997 (titled, Phenyl Glyoxamides as sPLA₂ Inhibitors), the entire disclosure of which is incorporated herein by reference.

The method of the invention is for treatment of a mammal, including a human, afflicted with renal dysfunction, said method comprising administering to said human a therapeutically effective amount a phenyl glyoxamide type sPLA₂ inhibitors useful in the method of the invention are as follows:

a compound of the formula (Ii)

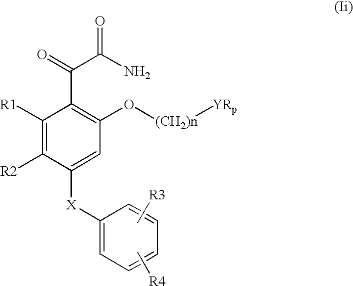

wherein:

X is —O— or —(CH₂)ₘ₋, where m is 0 or 1;

Y is —CO₂—, —PO₃—, —SO₃—;

R is independently —H or —(C₁-C₄)alkyl;

R¹ and R² are each independently —H, halo or —(C₁-C₄)alkyl;

R³ and R⁴ are each independently —H, —(C₁-C₄alkyl, (C₁-C₄)alkoxy, (C₁-C₄)alkylthio, halo, phenyl or phenyl substituted with halo;

N is 1-8; and

P is 1 when Y is —CO₂— or —SO₃— and 1 or 2 when Y is —PO₃—;.

or a pharmaceutically acceptable salt thereof.

A specific suitable phenyl glyoxamide type sPLA₂ inhibitors is 2-(4-carboxybut-1-yl-oxy)-4-(3-phenylphenoxy)phenylglyoxamide.

J) Pyrrole sPLA₂ inhibitors and methods of making them are disclosed in U.S. Patent Applicaton Ser. No. 08/985,518 filed Dec. 5, 1997 (titled, “Pyrroles as sPLA₂ Inhibitors”), the entire disclosure of which is incorporated herein by reference.

The method of the invention is for treatment of a mammal, including a human, afflicted with renal dysfunction, said method comprising administering to said human a therapeutically effective amount a pyrrole sPLA₂ inhibitors useful in the method of the invention as follows:

a compound of the formula (Ij)

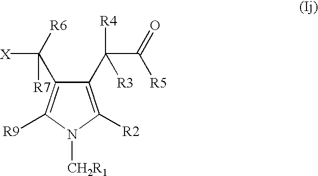

R¹ is hydrogen, (C₁-C₄)alkyl, phenyl or phenyl substituted with one or two substituents selected from the group consisting of —(C₁-C₄)alkyl, (C₁-C₄)alkoxy, phenyl(C₁-C₄)alkyl, (C₁-C₄)alkylthio, halo and phenyl;

R² is hydrogen, —(C₁-C₄)alkyl, halo, (C₁-C₄)alkoxy or (C₁-C₄)alkylthio;

R³ and R⁴ are each hydrogen or when taken together are ═O;

R⁵ is —NH₂ or —NHNH_{2;}

R⁶ and R⁷ are each hydrogen or when one of R⁶ and R⁷ is hydrogen, the other is —(C₁-C₄)alkyl, —(CH₂)ₙr¹⁰ where R¹⁰ is —CO₂R¹¹, —PO₃(R¹¹)₂, —PO₄(R¹¹)₂ or —SO₃R¹¹ where R¹¹ is independently hydrogen or —(C₁-C₄)alkyl and n is 0 to 4; or R⁶ and R⁷, taken together, are ═O or ═S;

X is R⁸(C₁-C₆)alkyl; R⁸(C₂-C₆)alkenyl or phenyl substituted at the ortho position with R⁸ where R⁸ is (CH₂)ₙr¹⁰ where R¹⁰ is —CO₂R¹¹, —PO₃(R¹¹)₂, —PO₄(R¹¹) or —SO₃R¹¹, R¹¹ and n is 1 to 4 as defined above, and additionally substituted with one or two substituents selected from the group consisting of hydrogen, —(C₁-C₄)alkyl, halo, (C₁-C₄)alkoxy, or two substituents which, when taken together with the phenyl ring to which they are attached, form a naphthyl group; and

R⁹ is hydrogen or methyl or ethyl;

or a pharmaceutically acceptable salt thereof.

Preferred pyrrole sPLA₂ inhibitors useful in the method of the invention are compounds of formula Ij wherein;

R¹ is phenyl;

R² is methyl or ethyl;

R⁵ is —NH₂;

R⁶ and R⁷ are each hydrogen;

X is R⁸(C₁-C₆)alkyl or phenyl substituted at the ortho position with R⁸ where

R⁸ is —CO₂R¹¹; and

R⁹ is methyl or ethyl.

A specific suitable pyrrole sPLA₂ inhibitors useful in the method of the invention is 2-[1-benzyl-2,5-dimethyl-4-(2-carboxyphenylmethyl)pyrrol-3-yl]glyoxamide.

K) Naphthyl glyoxamide sPLA₂ inhibitors and methods of making them are described in U.S. patent application Ser. No. 09/091,079, filed Dec. 9, 1966 (titled, “Naphthyl Glyoxamides as sPLA₂ Inhibitors”), the entire disclosure of which is incorporated herein by reference.

The method of the invention is for treatment of a mammal, including a human, afflicted with renal dysfunction, said method comprising administering to said human a therapeutically effective amount a naphthyl glyoxamide sPLA₂ inhibitors useful in the method of the invention are as follows:

a naphthyl glyoxamide compound or a pharmaceutically acceptable salt, solvate or prodrug derivative thereof; wherein said compound is represented by the formula Ik

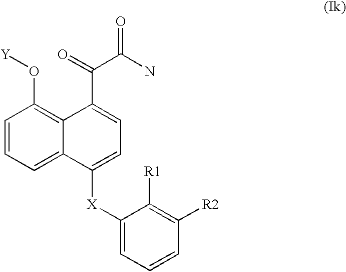

wherein:

R¹ and R² are each independently hydrogen or a non-interfering substituent with the proviso that at least one of R¹ or R² must be hydrogen;

X is —CH₂— or —O—; and

Y is (CH₂)ₙz where n is a number from 1-3 and Z is an acid group selected from the group consisting of CO₂H, —SO₃H or —PO(OH)₂.

A specific suitable naphthyl glyoxamide sPLA₂ inhibitors useful in the method of the invention has the following structural formula:

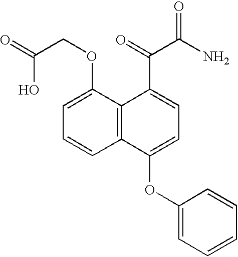

L) Phenyl acetamide sPLA₂ inhibitors and methods of making them are disclosed in U.S. patent application Ser. No. 08/976,858, filed Nov. 24, 1997 (titled, “Phenyl Acetamides as sPLA₂ Inhibitors”), the entire disclosure of which is incorporated herein by reference.

The method of the invention is for treatment of a mammal, including a human, afflicted with renal dysfunction, said method comprising administering to said human a therapeutically effective amount of a phenyl acetamide sPLA₂ inhibitor represented by formula (Il) as follows:

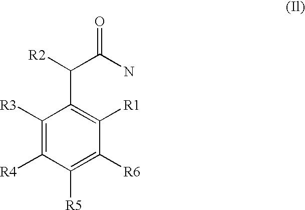

wherein:

R¹ is —H or —O(CH₂)ₙz;

R² is —H or —OH;

R³ and R⁴ are each independently —H, halo or —(C₁-C₄)alkyl;

one of R⁵ and R⁶ is —YR⁷ and the other is —H, where Y is —O— or —CH₂— and R⁷ is phenyl or phenyl substituted with one or two substituents selected from the group consisting of halo, —(C₁-C₄)alkyl, (C₁-C₄)alkoxy, phenyl or phenyl substituted with one or two halo groups;

Z is —CO₂R, —PO₃R₂ or —SO₃R where R is —H or —(C₁-C₄)alkyl; and

N is 1-8;

or a pharmaceutically acceptable salt, racemate or optical isomer thereof;

provided that when R⁶ is YR⁷, R¹ is hydrogen; and

when R¹, R², R³, R⁴ and R⁶ are hydrogen and R⁵ is YR⁷ where Y is —O—, R⁷ cannot be phenyl; and

when R¹, R², R³, R⁴ and R⁶ are hydrogen, R⁵ is YR⁷ where Y is CH₂, R⁷ cannot be phenyl substituted with one methoxy or two chloro groups.

Preferred suitable phenyl acetamide sPLA₂ inhibitors useful in the method of the invention are as follows:

compounds of formula I wherein R², R³ and R⁴ is H, Y is oxygen or CH₂, R⁷ is phenyl or phenyl substituted at the meta position with one or two substituents selected from halo, —(C₁-C₄)alkyl, (C₁-C₄)alkoxy, phenyl or phenyl substituted with halo and n is 4-5.

A specific suitable phenyl acetamide sPLA₂ inhibitors useful in the method of the invention is 2-(4-carboxybutoxy)-4-(3-phenylphenoxy)phenylacetamide.

M) Naphthyl acetamide sPLA₂ inhibitors and the method of making them are described in U.S. patent application Ser. No. 09/091,077, filed Dec. 9, 1996 (titled, “Benzyl naphthalene sPLA₂ Inhibitors”), the entire disclosure of which is incorporated herein by reference.

A naphthyl acetamide sPLA₂ inhibitor is represented by formula (Im) as follows:

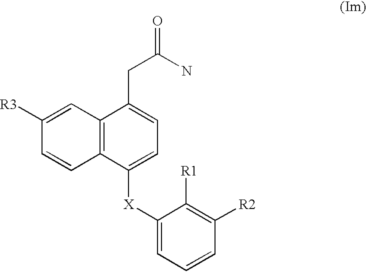

wherein:

R¹ and R² are each independently hydrogen or a non-interfering substituent with the proviso that at least one of R¹ and R² must be hydrogen;

R³ is hydrogen, —O(CH₂)ₙY,

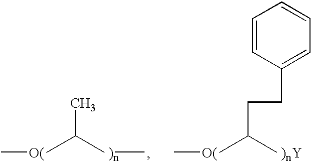

where n is from 2 to 4 and Y is —CO₂H, —PO₃H₂ or SO₃H; and

X is —O— or —CH₂—.

N) The method of the invention is for treatment of a mammal, including a human, afflicted with renal dysfunction, said method comprising administering to said human a therapeutically effective amount of pyrrolo[1,2-a]pyrazine derivative sPLA₂ inhibitors useful in the method of the invention as follows:

a compound of the formula (In)

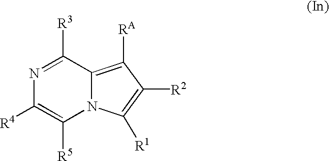

wherein R¹ is a group selected from (a) C6 to C20 alkyl, C6 to C20 alkenyl, C6 to C20 alkynyl, carbocyclic groups, and heterocyclic groups, (b) the groups represented by (a) each substituted independently with at least one group selected from non-interfering substituents, and (c) -(L¹)—R⁶ wherein L¹ is a divalent linking group of 1 to 18 atom(s) selected from hydrogen atom(s), nitrogen atom(s), carbon atom(s), oxygen atom(s), and sulfur atom(s), and R⁶ is a group selected from the groups (a) and (b);

R² is hydrogen atom, or a group containing 1 to 4 non-hydrogen atoms;

R³ is -(L²)-(acidic group) wherein L² is an acid linker having an acid linker length of 1 to 5;

R⁴ and R⁵ are selected independently from hydrogen atom, non-interfering substituents, carbocyclic groups, carbocyclic groups substituted with a non-interfering substituent(s), heterocyclic groups, and heterocyclic groups substituted by a non-interfering substituent(s) and;

R^{A} is a group represented by the formula:

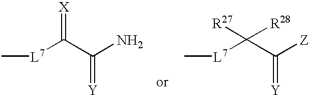

wherein L⁷ is a divalent linker group selected from a bond or a divalent group selected from —CH₂—, —O—, —S—, —NH—, or —CO—, R¹⁷ and R²⁸ are independently hydrogen atom, C1 to C3 alkyl or a halogen; X and Y are independently an oxygen atom or a sulfur atom; and Z is —NH₂ or —NHNH₂; the prodrugs thereof; or their pharmaceutically acceptable salts; or their solvates.

A preferred subclass of compounds of formula (In) are those where for R¹ the divalent linking group -(L₁)- is a group represented by any one of the following formulae (Ina) or (Inb) or (Inc):

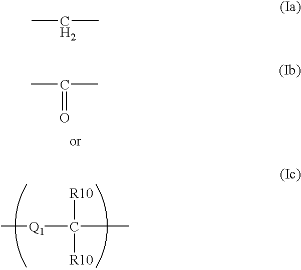

where Q₁ is a bond or any of the divalent groups (Ia) or (Ib) and each R¹⁰ is independently hydrogen, C₁₋₈ alkyl, C₁₋₈ haloalkyl or C₁₋₈ alkoxy.

Particularly preferred as the linking group -(L₁)- of R¹ is an alkylene chain of 1 or 2 carbon atoms, namely, —(CH₂)— or —(CH₂—CH₂)—.

Preferred sPLA₂ inhibitor compounds of the invention are represented by the formula (IIn):

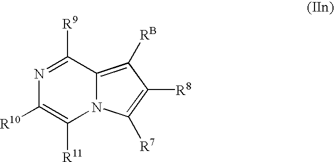

wherein R⁷ is —(CH₂)ₘ—R¹² wherein m is an integer from 1 to 6, and R¹² is (d) a group represented by the formula:

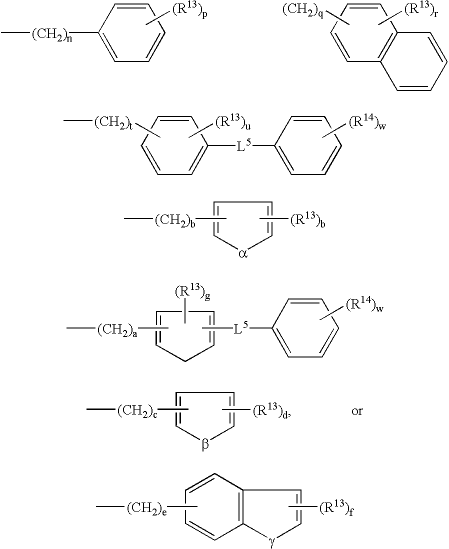

wherein a, c, e, n, q, and t are independently an integer from 0 to 2, R¹³ and R¹⁴ are independently selected from a halogen, C₁ to C₁₀alkyl, C₁ to C₁₀alkyloxy, C₁ to C₁₀ alkylthio, aryl, heteroaryl, and C₁ to C₁₀haloalkyl, a is an oxygen atom or a sulfur atom, L⁵ is a bond, —(CH₂)v-, —C═C—, —CC—, —O—, or —S—, v is an integer from 0 to 2, β is —CH₂— or —(CH₂)₂—, γ is an oxygen atom or a sulfur atom, b is an integer from 0 to 3, d is an integer from 0 to 4, f, p, and w are independently an integer from 0 to 5, r is an integer from 0 to 7, and u is an integer from 0 to 4, or is (e) a member of (d) substituted with at least one substituent selected from the group consisting of C₁ to C₆ alkyl, C₁ to C₆ alkyloxy, C₁ to C₆ haloalkyloxy, C₁ to C₆ haloalkyl, aryl, and a halogen;

R⁸ is C₁ to C₃ alkyl, C₃ to C₄ cycloalkyl, C₃ to C₄ cycloalkenyl, C₁ to C₂ haloalkyl, C₁ to C₃ alkyloxy, or C₁ to C₃ alkylthio;

R⁹ is -(L³)-R¹⁵ wherein L³ is represented by the formula:

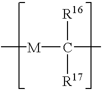

wherein M is —CH₂—, —O—, —N(R²⁴)—, or —S—, R¹⁶ and R¹⁷ are independently hydrogen atom, C₁ to C₁₀alkyl, aryl, aralkyl, alkyloxy, haloalkyl, carboxy, or a halogen, and R²⁴ is hydrogen atom or C₁ to C₆ alkyl, and R¹⁵ is represented by the formula:

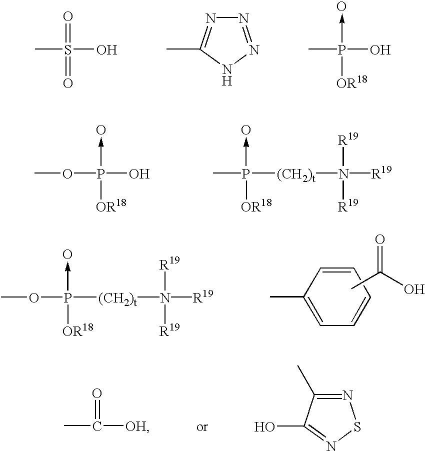

wherein R¹⁸ is hydrogen atom, a metal, or C₁ to C₁₀ alkyl, R¹⁹ is independently hydrogen atom, or C₁ to C₁₀ alkyl, and t is an integer from 1 to 8;

R¹⁰ and R¹¹ are independently hydrogen atom or a non-interfering substituent selected from hydrogen, C₁ to C₈ alkyl, C₁ to C₈ alkenyl, C₁ to C₈ alkynyl, C₇ to C₁₂ aralkyl, C₇ to C₁₂ alkaryl, C₃ to C₈ cycloalkyl, C₃ to C₈ cycloalkenyl, phenyl, tolyl, xylyl, biphenyl, C₁ to C₈ alkyloxy, C₂ to C₈ alkenyloxy, C₂ to C₈ alkynyloxy, C₂ to C₁₂ alkyloxyalkyl, C₂ to C₁₂ alkyloxyalkyloxy, C₂ to C₁₂ alkylcarbonyl, C₂ to C₁₂ alkylcarbonylamino, C₂ to C₁₂ alkyloxyamino, C₂ to C₁₂ alkyloxyaminocarbonyl, C₂ to C₁₂ alkylamino, C₁ to C₆ alkylthio, C₂ to C₁₂ alkylthiocarbonyl, C₁ to C₈ alkylsulfinyl, C₁ to C₈ alkylsulfonyl, C₂ to C₈ haloalkyloxy, C₁ to C₈ haloalkylsulfonyl, C₂ to C₈ haloalkyl, C₁ to C₈ hydroxyalkyl, —C(O)O(C₁ to C₈ alkyl), —(CH₂)_{z}—O—(C₁ to C₈ alkyl), benzyloxy, aryloxy, arylthio, —(CONHSO₂R²⁵), CHO, amino, amidino, halogen, carbamyl, carboxyl, carbalkoxy, —(CH₂)_{Z}—CO₂H, cyano, cyanoguanidinyl, guanidino, hydrazide, hydrazino, hydrazido, hydroxy, hydroxyamino, iodo, nitro, phosphono, —SO₃H, thioacetal, thiocarbonyl, or carbonyl, R²⁵ is C₁ to C₆ alkyl or aryl, z is an integer from 1 to 8; and R^{B} is a group represented by the formula:

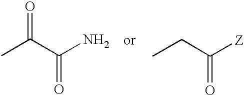

wherein Z is the same as defined above; the prodrugs thereof, or their pharmaceutically acceptable salts, or their solvates.

When the above b, d, f, p, r, u, and/or w are 2 or more, a plural number of R¹³ or R¹⁴ may be different from one another. When R¹³ is a substituent on the naphthyl group, the substituent may be substituted at any arbitrary position on the naphthyl group.

The invention further relates to specific preferred sPLA₂ inhibitor compounds of formule (I) or (II) namely a pyrrolo[1,2-a]pyrazine compound selected from the group consisting of:

[6-Benzyl-7-ethyl-8-oxamoylpyrrolo[1,2-a]pyrazin-1-yl]oxyacetic acid,

[6-Cyclohexylmethyl-7-ethyl-8-oxamoylpyrrolo[1,2-a]pyrazin-1-yl]oxyacetic acid,

[7-Ethyl-6-(3-methoxybenzyl)-8-oxamoylpyrrolo[1,2-a]pyrazin-1-yl]oxyacetic acid,

[6-(Benzo[b]thiophen-6-ylmethyl)-7-ethyl-8-oxamoylpyrrolo[1,2-a]pyrazin-1-yl]oxyacetic acid,

[6-Benzyl-7-ethyl-3-methyl-8-oxamoylpyrrolo[1,2-a]pyrazin-1-yl]oxyacetic acid,

[7-Ethyl-6-(4-fluorobenzyl)-3-methyl-8-oxamoylpyrrolo[1,2-a]pyrazin-1-yl]oxyacetic acid,

[6-(2-Biphenylmethyl)-7-ethyl-3-methyl-8-oxamoylpyrrolo[1,2-a]pyrazin-1-yl]oxyacetic acid,

[6-Cyclopentylmethyl-7-ethyl-3-methyl-8-oxamoylpyrrolo[1,2-a]pyrazin-1-yl]oxyacetic acid,

[6-(2-Benzyl)benzyl-7-ethyl-3-methyl-8-oxamoylpyrrolo[1,2-a]pyrazin-1-yl]oxyacetic acid,

[7-Ethyl-6-(2-(4-fluorophenyl)benzyl)-3-methyl-8-oxamoylpyrrolo[1,2-a]pyrazin-1-yl]oxyacetic acid,

[7-Ethyl-6-(3-fluorobenzyl)-3-methyl-8-oxamoylpyrrolo[1,2-a]pyrazin-1-yl]oxyacetic acid,

[6-Benzyl-7-ethyl-3-isopropyl-8-oxamoylpyrrolo[1,2-a]pyrazin-1-yl]oxyacetic acid,

[6-Benzyl-3,7-diethyl-8-oxamoylpyrrolo[1,2-a]pyrazin-1-yl]oxyacetic acid,

[6-Benzyl-7-ethyl-8-oxamoyl-3-phenylpyrrolo[1,2-a]pyrazin-1-yl]oxyacetic acid,

[6-Benzyl-7-ethyl-3-isobutyl-8-oxamoylpyrrolo[1,2-a]pyrazin-1-yl]oxyacetic acid,

[3,6-Dibenzyl-7-ethyl-8-oxamoylpyrrolo[1,2-a]pyrazin-1-yl]oxyacetic acid,

[7-Ethyl-3-methyl-8-oxamoyl-6-(2-(2-thienyl)benzyl)pyrrolo[1,2-a]pyrazin-1-yl]oxyacetic acid,

[7-Ethyl-3-methyl-8-oxamoyl-6-(2-phenylethynylbenzyl)pyrrolo[1,2-a]pyrazin-1-yl]oxyacetic acid,

[7-Ethyl-3-methyl-8-oxamoyl-6-(2-phenyloxybenzyl) pyrrolo[1,2-a]pyrazin-1-yl]oxyacetic acid,

[7-Ethyl-3-methyl-8-oxamoyl-6-(2-(3-thienyl)benzyl)pyrrolo[1,2-a]pyrazin-1-yl]oxyacetic acid,

[7-Ethyl-3-methyl-6-(2-(5-methylthien-2-yl)benzyl)-8-oxamoylpyrrolo[1,2-a]pyrazin-1-yl]oxyacetic acid,

[7-Ethyl-6-(2-(4-methoxyphenyl)benzyl)-3-methyl-8-oxamoylpyrrolo[1,2-a]pyrazin-1-yl]oxyacetic acid,

[7-Ethyl-3-methyl-6-(2-(4-methylphenyl)benzyl)-8-oxamoylpyrrolo[1,2-a]pyrazin-1-yl]oxyacetic acid,

[7-Ethyl-3-methyl-8-oxamoyl-6-(2-(2-phenylethyl)benzyl)pyrrolo[1,2-a]pyrazin-1-yl]oxyacetic acid,

[6-Benzyl-7-cyclopropyl-3-methyl-8-oxamoylpyrrolo[1,2-a]pyrazin-1-yl]oxyacetic acid,

[7-Cyclopropyl-6-(4-fluorobenzyl)-3-methyl-8-oxamoylpyrrolo[1,2-a]pyrazin-1-yl]oxyacetic acid,

[6-Benzyl-3-cyclohexyl-7-ethyl-8-oxamoylpyrrolo[1,2-a]pyrazin-1-yl]oxyacetic acid,

[6-(2-Biphenylmethyl)-3-cyclohexyl-7-ethyl-8-oxamoylpyrrolo[1,2-a]pyrazin-1-yl]oxyacetic acid,

[6-Benzyl-3,7-dimethyl-8-oxamoylpyrrolo[1,2-a]pyrazin-1-yl]oxyacetic acid,

[7-Ethyl-3-methyl-6-(5-methylthien-2-ylmethyl)-8-oxamoylpyrrolo[1,2-a]pyrazin-1-yl]oxyacetic acid,

[6-(Benzo[b]thiophen-3-ylmethyl)-7-ethyl-3-methyl-8-oxamoylpyrrolo[1,2-a]pyrazin-1-yl]oxyacetic acid,

Sodium [7-ethyl-6-(4-fluorobenzyl)-3-methyl-8-oxamoylpyrrolo[1,2-a]pyrazin-1-yl]oxyacetate,

Sodium [7-ethyl-6-(2-(4-fluorophenyl)benzyl)-3-methyl-8-oxamoylpyrrolo[1,2-a]pyrazin-1-yl]oxyacetate,

Sodium [7-ethyl-3-methyl-8-oxamoyl-6-(2-(2-thienyl)benzyl)pyrrolo[1,2-a]pyrazin-1-yl]oxyacetate,

Sodium [7-ethyl-3-methyl-8-oxamoyl-6-(2-(3-thienyl)benzyl)pyrrolo[1,2-a]pyrazin-1-yl]oxyacetate, and the prodrugs thereof; the parent acids thereof, or their pharmaceutically acceptable salts; or their solvates.

Most preferred as sPLA₂ inhibitors of the invention are

[7-ethyl-6-(2-(4-fluorophenyl)benzyl)-3-methyl-8-oxamoylpyrrolo[1,2-a]pyrazin-1-yl]oxyacetate, methyl ester;

[7-ethyl-6-(2-(4-fluorophenyl)benzyl)-3-methyl-8-oxamoylpyrrolo[1,2-a]pyrazin-1-yl]oxyacetate, ethyl ester;

[7-ethyl-6-(2-(4-fluorophenyl)benzyl)-3-methyl-8-oxamoylpyrrolo[1,2-a]pyrazin-1-yl]oxyacetate, morpholinylethyl ester;

[7-ethyl-6-(2-(4-fluorophenyl)benzyl)-3-methyl-8-oxamoylpyrrolo[1,2-a]pyrazin-1-yl]oxyacetate, sodium salt;

[7-ethyl-3-methyl-8-oxamoyl-6-(2-(2-thienyl)benzyl)pyrrolo[1,2-a]pyrazin-1-yl]oxyacetic acid, methyl ester;

[7-ethyl-3-methyl-8-oxamoyl-6-(2-(2-thienyl)benzyl)pyrrolo[1,2-a]pyrazin-1-yl]oxyacetic acid, ethyl ester;

[7-ethyl-3-methyl-8-oxamoyl-6-(2-(2-thienyl)benzyl)pyrrolo[1,2-a]pyrazin-1-yl]oxyacetic acid, morpholinylethyl ester;

[7-ethyl-3-methyl-8-oxamoyl-6-(2-(2-thienyl)benzyl)pyrrolo[1,2-a]pyrazin-1-yl]oxyacetic acid, sodium salt.

Formulations Suitable for use in the Method of the Invention

The sPLA₂ inhibitors used in the method of the invention may be administered to treat renal dysfunction by any means that produces contact of the active agent with the agent's site of action in the animal body. They can be administered by any conventional means available for use in conjunction with pharmaceuticals, either as individual therapeutic agents or in a combination of therapeutic agents. The sPLA₂ inhibitors can be administered alone, but are generally administered with a pharmaceutical carrier selected on the basis of the chosen route of administration and standard pharmaceutical practice.

Suitable formulations are those comprising a therapeutically effective amount of sPLA₂ inhibitor together with a pharmaceutically acceptable diluent or carrier, the composition being adapted for the particular route of administration chosen. By “pharmaceutically acceptable” it is meant the carrier, diluent or excipient must be compatible with the sPLA₂ inhibitor (“active compound”) in the formulation and not deleterious to the subject being treated.

For the pharmaceutical formulations any suitable carrier known in the art can be used. In such a formulation, the carrier may be a solid, liquid, or mixture of a solid and a liquid. A solid carrier can be one or more substances which may also act as flavoring agents, lubricants, solubilisers, suspending agents, binders, tablet disintegrating agents and encapsulating material.

Tablets for oral administration may contain suitable excipients such as calcium carbonate, sodium carbonate, lactose, calcium phosphate, together with disintegrating agents, such as maize, starch, or alginic acid, and/or binding agents, for example, gelatin or acacia, and lubricating agents such as magnesium stearate, stearic acid, or talc. In tablets the sPLA₂ inhibitor is mixed with a carrier having the necessary binding properties in suitable proportions and compacted in the shape and size desired. The powders and tablets preferably contain from about 0.01 to about 99 weight percent of the sPLA₂ inhibitor.

Sterile liquid form formulations include suspensions, emulsions, syrups and elixirs. The active compound can be dissolved or suspended in a pharmaceutically acceptable carrier, such as sterile water, saline, dextrose solution, sterile organic solvent or a mixture of both.

The active compound can be administered orally in solid dosage forms, such as capsules, tablets, and powders, or in liquid dosage forms, such as elixirs, syrups, and suspensions. It can also be administered parenterally, in sterile liquid dosage forms. It can also be administered by inhalation in the form of a nasal spray or lung inhaler. It can also be administered topically as an ointment, cream, gel, paste, lotion, solution, spray, aerosol, liposome, or patch. Dosage forms used to administer the active compound usually contain suitable carriers, diluents, preservatives, or other excipients, as described in Remington's Pharmaceutical Sciences, Merck Publishing Company, a standard reference text in the field.

Gelatin capsules may be prepared containing the active compound and powdered carriers, such as lactose, sucrose, mannitol, starch, cellulose derivatives, magnesium stearate, stearic acid, and the like. Similar diluents can be used to make compressed tablets and powders. Both tablets and capsules can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

Liquid dosage forms for oral administration can contain coloring and flavoring to increase patient acceptance.

For parenteral solutions, water, a suitable oil, saline, aqueous dextrose (glucose), and related sugar solutions and glycols such as propylene glycol or polyethylene glycols are suitable carriers for parenteral solutions. Solutions for parenteral administration contain the active compound, suitable stabilizing agents, and if necessary, buffer substances. Anti-oxidizing agents such as sodium bisulfite, sodium sulfite, or ascorbic acid either alone or combined are suitable stabilizing agents. Also used are citric acid and its salts and sodium EDTA. In addition, parenteral solutions can contain preservatives, such as benzalkonium chloride, methyl- or propyl-paraben, and chlorobutanol.

Topical ointments, creams, gels, and pastes contain with the active compound diluents such as waxes, paraffins, starch, polyethylene glycol, silicones, bentonites, silicic acid, animal and vegetable fats, talc and zinc oxide or mixtures of these or other diluents.

Topical solutions and emulsions can, for example, contain with the active compound, customary diluents (with the exclusion of solvents having a molecular weight below 200 except in the presence of a surface-active agent), such as solvents, dissolving agents and emulsifiers; specific examples are water, ethanol, 2-propanol, ethyl carbonate, benzyl alcohol, propylene glycol, oils, glycerol, and fatty acid esters of sorbitol or mixtures thereof. Compositions for topical dosing may also contain preservatives or anti-oxidizing agents.

Powders and sprays can contain along with the active compound, the usual diluents, such as lactose, talc, silicic acid, aluminum hydroxide, calcium silicate, and polyamide powders or mixtures of these materials. Aerosol sprays can contain the usual propellants. Liposomes can be made from such materials as animal or vegetable fats which will form lipid bilayers in which the active compound can be incorporated.

Formulations containing compounds of the invention may be administered through the skin by an appliance such as a transdermal patch. Patches can be made of a matrix such as polyacrylamide and a semipermeable membrane made from a suitable polymer to control the rate at which the material is delivered to the skin. Other suitable transdermal patch formulations and configurations are described in U.S. Pat. Nos. 5,296,222 and 5,271,940, the disclosures of which are incorporated herein by reference. Lipophilic prodrug derivatives of the sPLA₂ inhibitors are particularly well suited for transdermal absorption administration and delivery systems.

For all of the above formulations the preferred active compound are the 1H-indole-3-glyoxylamide compounds as previously described and methods of making as described in n U.S. Pat. No. 5,654,326 (the disclosure of which is incorporated herein by reference). Most preferred compounds within the general class of 1H-indole-3-glyoxylamides are ((3-(2-amino-1,2-dioxoethyl)-2-ethyl-1-(phenylmethyl)-1H-indol-4yl)oxy)acetic acid, sodium salt; and 1H-indole-3-glyoxylamides are ((3-(2-amino-1,2-dioxoethyl)-2-ethyl-1-(phenylmethyl)-1H-indol-4yl)oxy)acetic acid, methyl ester.

Proportion and Weight of Active Compounds used in the Method of the Invention

The 1H-indole-3-glyoxylamide compound may be used at a concentration of 0.1 to 99.9 weight percent of the formulation.

Preferably the pharmaceutical-formulation is in unit dosage form. The unit dosage form can be a capsule or tablet itself, or the appropriate number of any of these. The quantity of active compound in a unit dose of composition may be varied or adjusted from about 0.1 to about 1000 milligrams or more according to the particular treatment involved.

Compositions (dosage forms) suitable for internal administration contain from about 1 milligram to about 500 milligrams of active compound per unit. In these pharmaceutical compositions the active compound will ordinarily be present in an amount of about 0.5-95% by weight based on the total weight of the composition.

Examples of useful pharmaceutical compositions and their proportions of ingredients are illustrated as follows:

Capsules: Capsules may be prepared by filling standard two-piece hard gelatin capsules each with 50 mg of powdered active compound, 175 mg of lactose, 24 mg of talc, and 6 mg of magnesium stearate. Soft Gelatin Capsules: A mixture of active compound in soybean oil is prepared and injected by means of a positive displacement pump into gelatin to form soft gelatin capsules containing 50 mg of the active compound. The capsules are washed in petroleum ether and dried. Tablets: Tablets may be prepared by conventional procedures so that the dosage unit is 50 mg of active compound, 6 mg of magnesium stearate, 70 mg of microcrystalline cellulose, 11 mg of cornstarch, and 225 mg of lactose. Appropriate coatings may be applied to increase palatability or delay absorption.

Suspensions: An aqueous suspension is prepared for oral administration so that each 5 ml contain 25 mg of finely divided active compound, 200 mg of sodium carboxymethyl cellulose, 5 mg of sodium benzoate, 1.0 g of sorbitol solution, U.S.P., and 0.025 mg of vanillin. Injectables: A parenteral composition suitable for administration by injection is prepared by stirring 1.5% by weight of active compound in 10% by volume propylene glycol and water. The solution is sterilized by commonly used techniques.

Nasal Spray: An aqueous solution is prepared such that each 1 ml contains 10 mg of active compound, 1.8 mg methylparaben, 0.2 mg propylparaben and 10 mg methylcellulose. The solution is dispensed into 1 ml vials.

The active compound may be used at a concentration of 0.01 to 99.9 weight percent of the formulation.

Aerosol formulations are capable of dispersing into particle sizes of from about 0.5 to about 10 microns and have sufficient sPLA₂ inhibitor to achieve concentrations of the inhibitor on the airway surfaces of from about 10⁻¹⁰ to 10⁻² moles per liter.

The Practice of the Method of the Invention

The use of sPLA₂ inhibitors in the method of the invention prevents progressive deterioration by inhibiting or reducing the degree of renal dysfunction that may be a primary pathologic process in renal dysfunction. The method of the invention is preferably used early in the symptomatic-life of the patient afflicted with renal dysfunction.

The method of the invention can be practiced using pharmaceutical formulations containing sPLA₂ inhibitors (preferably, sPLA₂ inhibitors identified as preferred herein) or formulations containing such sPLA₂ inhibitors as taught in the preceding section.

Although it is believed that the underlying causes of renal dysfunction will not be prevented by the method of this invention, the symptoms will be reduced in severity or extent by administration of sPLA₂ inhibitors (and their formulations).

The dosage administered will vary depending upon known factors such as the pharmacodynamic characteristics of the particular agent, and its mode and route of administration; age, health, and weight of the recipient; nature and extent of symptoms, kind of concurrent treatment, frequency of treatment, and the effect desired. Usually a daily dosage of active compound can be about 0.1 to 200 milligrams per kilogram of body weight. Ordinarily 0.5 to 50, and preferably 1 to 25 milligrams per kilogram per day given in divided doses 1 to 6 times a day or in sustained release form is effective to obtain desired results.

In general, the sPLA₂ inhibitor will be administered to an animal so that a therapeutically effective amount is received. A therapeutically effective amount may conventionally be determined for an individual patient by administering the active compound in increasing doses and observing the effect on the patient, for example, improvement in exercise, increased appetite, or a reduction in other symptoms associated with renal dysfunction.

Generally, the compound must be administered in a manner and a dose to achieve in the animal a blood level concentration of sPLA₂ inhibitor of from 10 to 3000 nanograms/ml, and preferably a concentration of 100 to 800 nanograms/ml.

The treatment regimen may stretch over many days to months or to years. Oral dosing is preferred for patient convenience and tolerance. With oral dosing, one to four oral doses per day, each from about 0.01 to 25 mg/kg of body weight with preferred doses being from about 0.1 mg/kg to about 2 mg/kg.

Parenteral administration (particularly, intravenous administration) is often preferred in instances where rapid alleviation of patient distress is required. With parenteral administration doses of 0.01 to 100 mg/kg/day administered continuously or intermittently throughout the day may be used. For parenteral administration, the compound may be administered in a physiologic saline vehicle (e.g., 0.9% normal saline, 0.45% normal saline, etc.) a dextrose vehicle (e.g., 5% dextrose in water), or a combination of saline and dextrose vehicle (0.9% normal saline in 5% dextrose).

Inhalation therapy also may be useful either alone or as an adjunct to other routes of administration. With inhalation therapy, doses necessary to produce a decrease in the clinical symptoms of renal dysfunction are readily determined and used.

Testing Methods for Renal Dysfunction

The diagnostic criteria for renal dysfunction are those found in standard medical references (e.g., Harrison's Principles of Internal Medicine, thirteenth ed., 1994, by McGraw-Hill, Inc., ISBN 0-07-032370-4). These criteria, or criteria designated by competent medical opinion may be used to determine when to begin using the method of the invention, the frequency and degree of treatment, and the time for cessation of treatment.

For example, the renal dysfunction patient having renal disease may be evaluated with any conventional measure of renal capacity.

The renal dysfunction patient having gastrointestinal disease may be evaluated by conventional criteria for adequate nutrition.

The underlying renal dysfunction event may be evaluated and treated according to current standards of good medical practice, where the standard treatment is supplemented with the administration of a compound according to this invention.

### Results of a To Evaluate the Effects of Compound 374388 in Fischer 344 Rats with Surgically Induced Chronic Renal Failure

Study R⁰⁹¹⁹⁹

374388 is an oralsPLA₂ inhibitor compound of the present invention. The purpose of this study was to evaluate the effect of 374388 in a model of chronic renal failure in male Fisher 344 (F344) rats, when administered daily by gavage. Doses of 0, 3 or 30 mg/kg were administered for approximately 2 months (dosing begun approximately 60 days after the nephrectomy performed at Lilly Research Laboratories). Alterations in live phase, and morphologic and clinical pathology parameters were compared to findings in age-matched control, sham-nephrectomized control, and vehicle-control nephrectomized rats.

No compound-related alterations suggestive of toxicity occurred.

Chronic renal failure was successfully induced in nephrectomized rats. The only remarkable difference in mortality among the nephrectomized groups was an increased number of rats in the 30 mg/kg group that died during the pretreatment phase. The high pretreatment mortality in this group was likely due to the increased area of renal infarction seen in most rats in the 30-mg/kg group at necropsy, resulting in a more rapid onset of renal failure. Due to the mortality in this group, data from this group were not analyzed statistically. Body weight and body weight-gains were improved in uremic rats given 3 mg/kg 374388 compared to nephrectomized controls. Near the end of the treatment phase (Day 112), mean body weight for rats given 3 mg/kg was significantly increased relative to the nephrectomized control value and was within approximately 5% of the control group mean, as compared to 19% for nephrectomized control rats. Although not statistically analyzed, body weight change data for rats given 30 mg/kg followed the same general trend as that seen in the 3-mg/kg group.

Alterations in clinical and morphologic pathology parameters in nephrectomized rats were attributed to the model (chronic renal failure-uremia). Alterations in these parameters, in general, were less pronounced in nephrectomized rats given 374388, suggesting that this compound may ameliorate the effects of uremia. On Day 117, nephrectomized rats given 3 mg/kg had minimal increases in cholesterol, total protein, and total carbon dioxide and slight to moderate decreases in BUN, CREAT and IP compared to nephrectomized controls. Nephrectomized control rats surviving to study termination had more severe nephropathy, and a higher incidence of secondary parathyroid hyperplasia, fibrous osteodystrophy and periarteritis nodusus, compared to nephrectomized rats given 3 mg/kg.

In conclusion, this study demonstrated a reliable model of chronic renal failure in the rat. Data from this preliminary study suggest that daily doses of 3 mg/kg of 374388 ameliorated the effects of uremia in rats with surgically induced chronic renal failure.

Therefore, 374388 and other members of the class of compounds of formula I may be potential therapeutic agents for the treatment of chronic renal failure (uremia) in human beings.

While the present invention has been illustrated by certain specific embodiments, these are not intended to limit the scope of the invention.

1. A method for treatment of an animal afflicted with renal dysfunction, said method comprising administering to said animal a therapeutically effective amount of a composition including members selected from the group comprising: 1H-indole-3-glyoxylamide, 1H-indole-3-hydrazide, 1H-indole-3-acetamide, 1H-indole-1-glyoxylamide, 1H-indole-1-hydrazide, 1H-indole-1-acetamide, indolizine-1-acetamide, indolizine-1-acetic acid hydrazide, indolizine-1-glyoxylamide, indene-1-acetamide, indene-1-acetic acid hydrazide, indene-1-glyoxylamide, carbazole, tetrahydrocarbazole, pyrazole, phenyl glyoxamide, pyrrole, naphthyl glyoxamide, naphthyl acetamide, phenyl acetamide, 9H-carbazole, 9-benzylcarbazole and mixtures thereof.

2. A method for treatment of an animal afflicted with renal dysfunction, said method comprising administering to said animal a therapeutically effective amount of a 1H-indole-3-glyoxylamide represented by the formula (I), or a pharmaceutically acceptable salt or aliphatic ester prodrug derivative thereof; 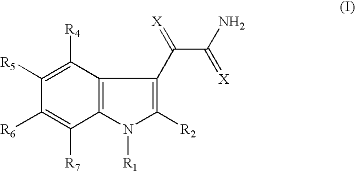
where;
X is oxygen,
R¹ is selected from the group consisting of —C₇-C₂₀ alkyl, 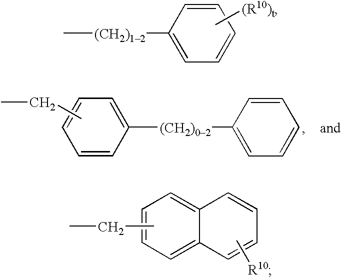
where
R¹⁰ is selected from the group consisting of halo, C₁-C₁₀ alkyl, C₁-C₁₀ alkoxy, —S—(C₁-C₁₀ alkyl) and halo(C₁-C₁₀)alkyl, and t is an integer from 0 to 5 both inclusive;
R₂ is selected from the group consisting of hydrogen, halo, cyclopropyl, methyl, ethyl, and propyl;
R₄ and R₅ are independently selected from the group consisting of hydrogen, a non-interfering substituent and the group, -(Lₐ)-(acidic group); where, at least one of R₄ and R₅ is the group, -(Lₐ)-(acidic group) and wherein the (acidic group) is selected from the group consisting of —CO₂H, —SO₃H, or —P(O)(OH)₂; where,
-(Lₐ)- is an acid linker with the proviso that; the acid linker group, -(Lₐ)-, for R₄ is selected from the group consisting of 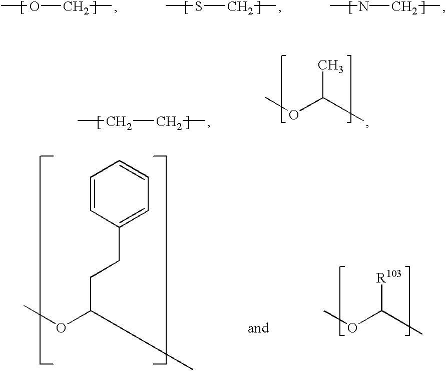
where R¹⁰³ is a non-interfering substituent, and where, the acid linker, -(Lₐ)-, for R⁵ is selected from the group consisting of 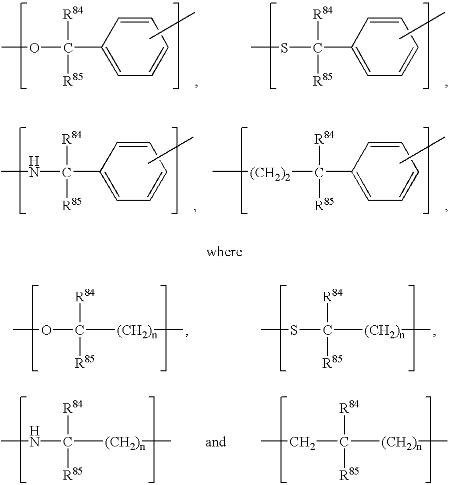
R⁸⁴ and R⁸⁵ are each independently selected from hydrogen, C₁-C₁₀ alkyl, aryl, C₁-C₁₀ alkaryl, C₁-C₁₀ arylkyl, carboxy, carbalkoxy, and halo where n is between 1 and 8 and,
R₆ and R₇ are each independently selected from hydrogen and non-interfering substituents,
where non-interfering substituents are selected from the group consisting of C₁-C₆ alkyl, C₂-C₆ alkenyl, C₂-C₆ alkynyl, C₇-C₁₂ arylenalkyl, C₇-C₁₂ alkaryl, C₃-C₈ cycloalkyl, C₃-C₈ cycloalkenyl, phenyl, tolulyl, xylenyl, biphenyl, C₁-C₆ alkoxy, C₂-C₆ alkenyloxy, C₂-C₆ alkynyloxy, C₂-C₁₂ alkoxyalkyl, C₂-C₁₂ alkoxyalkyloxy, C₂-C₁₂ alkylcarbonyl, C₂-C₁₂ alkylcarbonylamino, C₂-C₁₂ alkoxyamino, C₂-C₁₂ alkoxyaminocarbonyl, C₂-C₁₂ alkylamino, C₁-C₆ alkylthio, C₂-C₁₂ alkylthiocarbonyl, C₁-C₆ alkylsulfinyl, C₁-C₆ alkylsulfonyl, C₂-C₆ haloalkoxy, C₁-C₆ haloalkylsulfonyl, C₂-C₆ haloalkyl, C₁-C₆ hydroxyalkyl, —C(O)O(C₁-C₆ alkyl), —(CH₂)ₙ—O—(C₁-C₆ alkyl), benzyloxy, phenoxy, phenylthio, —(CONHSO₂R), —CHO, amino, amidino, bromo, carbamyl, carboxyl, carbalkoxy, —(CH₂)ₙ—CO₂H, chloro, cyano, cyanoguanidinyl, fluoro, guanidino, hydrazide, hydrazino, hydrazido, hydroxy, hydroxyamino, iodo, nitro, phosphono, —SO₃H, thioacetal, thiocarbonyl, and C₁-C₆ carbonyl.

3. A method for treatment of an animal afflicted with renal dysfunction, said method comprising administering to said animal a therapeutically effective amount of a compound represented by the formula (II), or a pharmaceutically acceptable salt or aliphatic ester prodrug derivative thereof; 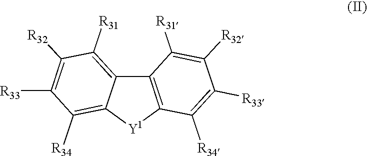
where Y₁ is selected from the group consisting of O, NH, NR₁ and S;
R¹ is selected from the group consisting of —C₇-C₂₀ alkyl, 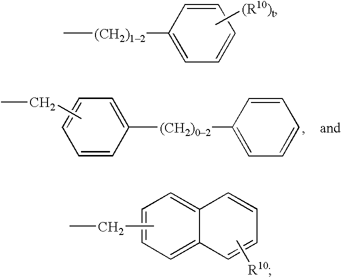
where
R¹⁰ is selected from the group consisting of halo, C₁-C₁₀ alkyl, C₁-C₁₀ alkoxy, —S—(C₁-C₁₀ alkyl) and halo(C₁-C₁₀)alkyl, and t is an integer from 0 to 5 both inclusive;
where R₃₁, R₃₂, R₃₃, R_{31′}, R_{32′}, R_{33′}, R₃₄ and R₃₄, are independently selected from the group consisting of hydrogen, CONR¹⁰¹R¹⁰², alkyl, alkylaryl, aryl, alkylheteroaryl, haloalkyl, alkylCONR¹⁰¹R¹⁰², a non-interfering substituent and the group -(Lₐ)-(acidic group);
where at least one of R₃₁, R₃₂, R₃₃ or R₃₄ is the group -(Lₐ)-(acidic group)
where -(Lₐ)- is an acid linker selected from the group consisting of 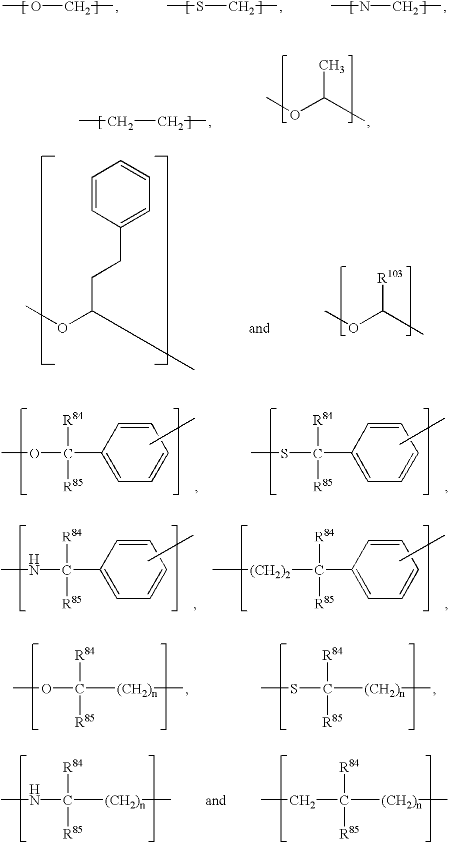
where R⁸⁴ and R⁸⁵ are each independently selected from the group consisting of hydrogen, C₁-C₁₀ alkyl, aryl, C₁-C₁₀ alkaryl, C₁-C₁₀ aralkyl, carboxy, carbalkoxy, and halo; and n is 1 or 2 and,
where the (acidic group) is selected from the group consisting of —CO₂H, —SO₃H, —CO₂NR¹⁰¹R¹⁰² and —P(O)(OH)₂ and,
where R¹⁰¹ and R¹⁰² are independently selected from the group consisting of hydrogen, alkyl, aryl, heteroaryl and haloalkyl and,
where non-interfering substituents are selected from the group consisting of C₁-C₆ alkyl, C₂-C₆ alkenyl, C₂-C₆ alkynyl, C₇-C₁₂ arylalkyl, C₇-C₁₂ alkylaryl, C₃-C₈ cycloalkyl, C₃-C₈ cycloalkyl, phenyl, tolulyl, xylyl, biphenyl, C₁-C₆ alkoxy, C₂-C₆ alkyloxy, C₂-C₆ alkynyloxy, C₂-C₁₂ alkoxyalkyl, C₂-C₁₂ alkoxyalkyloxy, C₂-C₁₂ alkylcarbonyl, C₂-C₁₂ alkylcarbonylamino, C₂-C₁₂ alkoxyamino, C₂-C₁₂ alkoxyaminocarbonyl, C₂-C₁₂ alkylamino, C₁-C₆ alkylthio, C₂-C₁₂ alkylthiocarbonyl, C₁-C₆ alkylsulfinyl, C₁-C₆ alkylsulfonyl, C₂-C₆ haloalkoxy, C₁-C₆ haloalkylsulfonyl, C₂-C₆ haloalkyl, C₁-C₆ hydroxyalkyl, —C(O)O(C₁-C₆ alkyl), —(CH₂)ₙ—O—(C₁-C₆ alkyl), benzyloxy, phenoxy, phenylthio, —(CONHSO₂(R)), —CHO, amino, amidino, bromo, carbamyl, carboxyl, carbalkoxy, —(CH₂)ₙ—CO₂H, chloro, cyano, cyanoguanidinyl, fluoro, guanidino, hydrazide, hydrazino, hydrazido, hydroxy, hydroxyamino, iodo, nitro, phosphono, —SO₃H, thioacetal, thiocarbonyl, and C₁-C₆ carbonyl and where n is between about 1 and 8 and,
R is selected from the group consisting of hydrogen and alkyl.

4. A method for treatment of an animal afflicted with renal dysfunction, said method comprising administering to said animal in need of such treatment, a therapeutically effective amount of a 1H-indole-3-glyoxylamide compound or a 9H-carbazole or a pharmaceutically acceptable salt, solvate, or a prodrug derivative thereof selected from the group consisting of compounds (A) through (AL):
(A) [[3-(2-amino-1,2-dioxoethyl)-2-methyl-1-(phenylmethyl)-1H indol-4-yl]oxy]acetic acid,
(B) dl-2-[[3-(2-amino-1,2-dioxoethyl)-2-methyl-1-(phenylmethyl)-1H indol-4-yl]oxy]propanoic acid,
(C) [[3-(2-amino-1,2-dioxoethyl)-1-([1,1′-biphenyl]-2-ylmethyl)-2 methyl-1H-indol-4-yl]oxy]acetic acid,
(D) [[3-(2-amino-1,2-dioxoethyl)-1-([1,1-biphenyl]-3-ylmethyl)-2-methyl-1H-indol-4-yl]oxy]acetic acid,
(E) [[3-(2-amino-1,2-dioxoethyl)-1-([1,1′-biphenyl]-4-ylmethyl)-2-methyl-1H-indol-4-yl]oxy]acetic acid,
(F) [[3-(2-amino-1,2-dioxoethyl)-1-[(2,6-dichlorophenyl)methyl]-2-methyl-1H-indol-4-yl]oxy]acetic acid
(G) [[3-(2-amino-1,2-dioxoethyl)-1-[4(-fluorophenyl)methyl]-2-methyl-1H-indol-4-yl]oxy]acetic acid,
(H) [[3-(2-amino-1,2-dioxoethyl)-2-methyl-1-[(1-naphthalenyl)methyl]-1H-indol-4-yl]oxy]acetic acid,
(I) [[3-(2-amino-1,2-dioxoethyl)-2-ethyl-1-(phenylmethyl)-1H-indol-4-yl]oxy]acetic acid,
(J) [[3-(2-amino-1,2-dioxoethyl)-1-[(3-chlorophenyl)methyl]-2-ethyl-1H-indol-4-yl]oxy]acetic acid,
(K) [[3-(2-amino-1,2-dioxoethyl)-1-([1,1′-biphenyl]-2-ylmethyl)-2-ethyl-1H-indol-4-yl]oxy]acetic acid,
(L) [[3-(2-amino-1,2-dioxoethyl)-1-([1,1-biphenyl]-2-ylmethyl)-2-propyl-1H-indol-4-yl]oxy]acetic acid,
(M) [[3-(2-amino-1,2-dioxoethyl)-2-cyclopropyl-1-(phenylmethyl)-1H-indol-4-yl]oxy]acetic acid,
(N) [[3-(2-amino-1,2-dioxoethyl)-1-([1,1′-biphenyl]-2-ylmethyl)-2-cyclopropyl-1H-indol-4-yl]oxy]acetic acid,
(O) 4-[[3-(2-amino-1,2-dioxoethyl)-2-ethyl-1-(phenylmethyl)-1H-indol-5-yl]oxy]butanoic acid,
(AG) 1-(9H-benzylcarbazol-1-halo-4-yloxy-5-alkylamido) alkylacetate,
(AH) 1-(9H-benzylcarbazol-4-yloxy-5-alkylamido) alkylacetate,
(AI) 1-(9H-benzylcarbazol-1-halo-4-yloxy-5-alkylamido) acetic acid,
(AJ) 1-(9H-benzylcarbazol-4-yloxy-5-alkylamido) acetic acid and
(AK) mixtures of (AG) through (AJ) and
(AL) mixtures of (A) through (AK) combined with an additional treatment composition.

5. A method for treatment of an animal afflicted with renal dysfunction, said method comprising administering to said animal in need of such treatment a therapeutically effective amount of a composition selected from the group comprising: 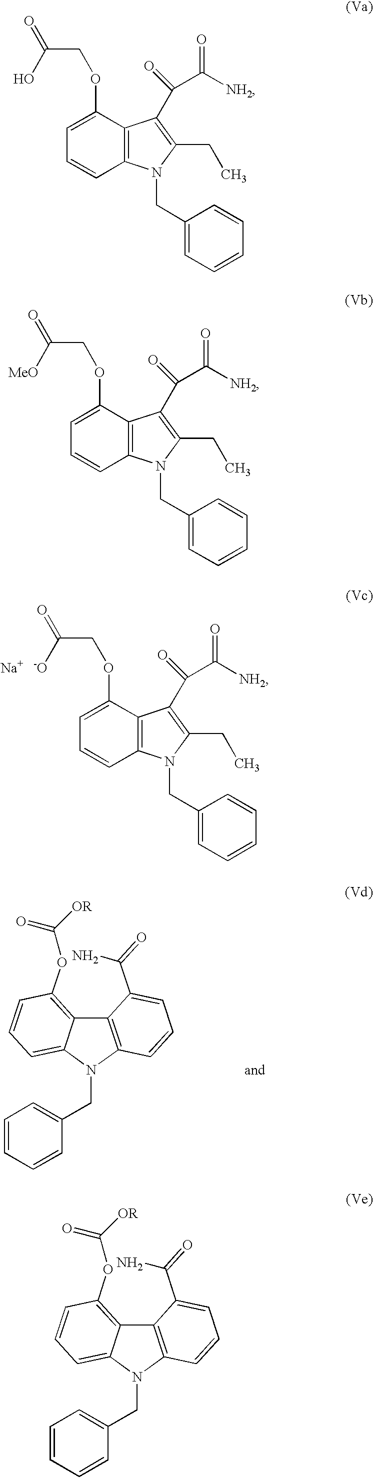
where R is independently selected from the group consisting of hydrogen, alkyl, aryl and heteroaryl.
R¹⁰⁵ is selected from the group consisting of NH₂, NHNH₂ and alkylamino and,
R¹¹ is selected from groups (a), (b) and (c) where;
(a) is C₇-C₂₀ alkyl, C₇-C₂₀ alkenyl, C₇-C₂₀ alkynyl, carbocyclic radical, or heterocyclic radical, or
(b) is a member of (a) substituted with one or more independently selected non-interfering substituents; or
(c) is the group -(L)- R⁸⁰; where, -(L)- is a divalent linking group of 1 to 12 atoms and where R⁸⁰ is a group selected from (a) or (b);
R² is hydrogen, halo, C₁-C₃ alkyl, C₃-C₄ cycloalkyl, C3C4 cycloalkenyl, —O—(C₁-C₂ alkyl), —S—(C₁-C₂ alkyl), or a non-interfering substituent having a total of 1 to 3 atoms other than hydrogen;
R¹⁶ and R¹⁷ are independently selected from hydrogen, a non-interfering substituent, or the group, -(Lₐ)-(acidic group); wherein -(Lₐ)-, is an acid linker having an acid linker length of 1 to 10; provided, that at least one of R¹⁶ and R¹⁷ must be the group, -(Lₐ)-(acidic group); and
R¹⁴ and R¹⁵ are each independently selected from hydrogen, non-interfering substituents, carbocyclic radical, carbocyclic radical substituted with non-interfering substituents, heterocyclic radical, and heterocyclic radical substituted with non-interfering substituents.

6. A method for treatment of an animal afflicted with renal dysfunction, said method comprising administering to said animal in need of such treatment, a therapeutically effective amount of a compound of the formula (XII) 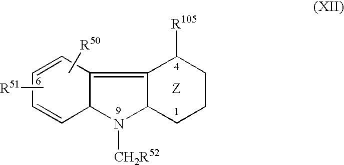
where:
R¹⁰⁵ is selected from the group consisting of NH₂, NHNH₂ and alkylamino and,
R⁵⁰ is —OH, or —O(CH₂)ₘ R⁵³ where
R⁵³ is selected from the group consisting of H, —CO₂H, —CO₂(C₁-C₄ alkyl), 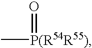
phenyl, —CO₂H substituted phenyl and —CO₂(C₁-C₄ alkyl) where
R⁵⁴ and R⁵⁵ are each independently selected from the group consisting of —OH and —O(C₁-C₄ alkyl) and,
m is 1, 2 or 3;
R⁵¹ is selected from the group consisting of H, —O(C₁-C₄ alkyl), and —(CH₂)ₙR⁵⁶ where
R⁵⁶ is selected from the group consisting of H, —N R⁵⁷R⁵⁸, 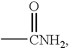
—CN,
and phenyl where,
R⁵⁷ and R⁵⁸ are independently selected from the group consisting of —(C₁-C₄)alkyl, and phenyl(C₁-C₄)alkyl and,
n is between about 0 and 9;
R⁵² is selected from the group consisting of H, —(C₅-C₁₄)alkyl, —(C₃-C₁₄)cycloalkyl, phenyl, or phenyl substituted with 1 or 2 substituents selected from the group consisting of —(C₁-C₄)alkyl, (C₁-C₄)alkoxy, phenyl(C₁-C₄)alkyl, (C₁-C₄)alkylthio, halo or phenyl; and
Z is cyclohexenyl or phenyl;
or a pharmaceutically acceptable salt, racemate or optical isomer thereof;
provided that when R⁵¹ is H, R⁵² is phenyl, m is 1 or 2 and R⁵⁰ is a substituent at the 6 position, R⁵³ cannot be H; and
when R¹⁰⁵ is NHNH², R⁸ cannot be

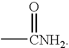

7. A method for treatment of an animal afflicted with renal dysfunction, wherein the method comprises administering to said animal in need of such treatment, a therapeutically effective amount of a compound selected from the group consisting of; 4-[(9-benzyl-4-carbamoyl-1,2,3,4-tetrahydrocarbazol-6-yl)oxy]butyric acid; 3-[(9-benzyl-4-carbamoyl-1,2,3,4-tetrahydrocarbazol-6-yl)oxy]propylphosphonic acid; 2-[(9-benzyl-4-carbamoyl-1,2,3,4-tetrahydrocarbazol-6-yl)oxy]methylbenzoic acid;
3-[(9-benzyl-4-carbamoyl-7-n-octyl-1,2,3,4-tetrahydrocarbazol-6-yl)oxy]propylphosphonic acid; 4-[(9-benzyl-4-carbamoyl-7-ethyl-1,2,3,4-tetrahydrocarbazol-6-yl)oxy]butyric acid; 3-[(9-benzyl-4-carbamoyl-7-ethyl-1,2,3,4-tetrahydrocarbazol-6-yl)oxy]propylphosphonic acid; 3-[(9-benzyl-4-carbamoyl-7-ethyl-1,2,3,4-tetrahydrocarbazol-6-yl)oxy]propylphosphonic acid; (S)-(+)-4-[(9-benzyl-4-carbamoyl-7-ethyl-1,2,3,4-tetrahydrocarbazol-6-yl)oxy]butyric acid; 4-[9-benzyl-4-carbamoyl-6-(2-cyanoethyl)-1,2,3,4-tetrahydrocarbazol-6-yl]oxybutyric acid; 4-[9-benzyl-4-carboxamido-7-(2-phenylethyl)-1,2,3,4-tetrahydrocarbazol-6-yl]oxybutyric acid; and 4-[9-benzyl-4-carboxamidocarbazol-6-yl]oxybutyric acid.

8. A method for treatment of an animal afflicted with renal dysfunction, wherein the method comprises administering to said animal in need of such treatment, a therapeutically effective amount of a compound represented by the formula (In): 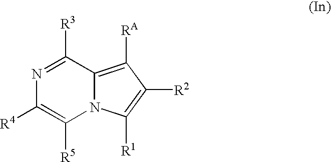
wherein R¹ is a group selected from (a) C₆ to C₂₀ alkyl, C₆ to C₂₀ alkenyl, C₆ to C₂₀ alkynyl, carbocyclic. groups, and heterocyclic groups, (b) the groups represented by (a) each substituted independently with at least one group selected from non-interfering substituents, and (c) -(L¹)—R⁶ wherein L¹ is a divalent linking group of 1 to 18 atom(s) selected from hydrogen atom(s), nitrogen atom(s), carbon atom(s), oxygen atom(s), and sulfur atom(s), and R⁶ is a group selected from the groups (a) and (b);
R² is hydrogen atom, or a group containing 1 to 4 non-hydrogen atoms;
R³ is -(L²)-(acidic group) wherein L² is an acid linker having an acid linker length of 1 to 5;
R⁴ and R⁵ are selected independently from hydrogen atom, non-interfering substituents, carbocyclic groups, carbocyclic groups substituted with a non-interfering substituent(s), heterocyclic groups, and heterocyclic groups substituted by a non-interfering substituent(s); and
R^{A} is a group represented by the formula: 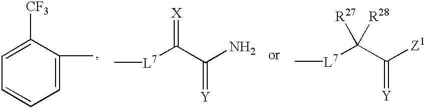
wherein L⁷ is a divalent linker group selected from a bond or a divalent group selected from —CH₂—, —O—, —S—, —NH—, or —CO—, R²⁷ and R²⁸ are independently hydrogen atom, C₁ to C₃ alkyl or a halogen; X and Y are independently an oxygen atom or a sulfur atom; and Z is —NH₂ OR —NHNH₂; the prodrugs thereof; or their pharmaceutically acceptable salts; or their solvates.

9. A method for treatment of an animal afflicted with renal dysfunction, wherein the method comprises administering to said animal in need of such treatment, a therapeutically effective amount of a pyrrolo[1,2-a]pyrazine compound selected from the group consisting of:
[7-ethyl-6-(2-(4-fluorophenyl)benzyl)-3-methyl-8-oxamoylpyrrolo[1,2-a]pyrazin-1-yl]oxyacetate, methyl ester;
[7-ethyl-6-(2-(4-fluorophenyl)benzyl)-3-methyl-8-oxamoylpyrrolo[1,2-a]pyrazin-1-yl]oxyacetate, ethyl ester;
[7-ethyl-6-(2-(4-fluorophenyl)benzyl)-3-methyl-8-oxamoylpyrrolo[1,2-a]pyrazin-1-yl]oxyacetate, morpholinylethyl ester;
[7-ethyl-6-(2-(4-fluorophenyl)benzyl)-3-methyl-8-oxamoylpyrrolo[1,2-a]pyrazin-1-yl]oxyacetate, sodium salt;
[7-ethyl-3-methyl-8-oxamoyl-6-(2-(2-thienyl)benzyl)pyrrolo[1,2-a]pyrazin-1-yl]oxyacetic acid, methyl ester;
[7-ethyl-3-methyl-8-oxamoyl-6-(2-(2-thienyl)benzyl)pyrrolo[1,2-a]pyrazin-1-yl]oxyacetic acid, ethyl ester;
[7-ethyl-3-methyl-8-oxamoyl-6-(2-(2-thienyl)benzyl)pyrrolo[1,2-a]pyrazin-1-yl]oxyacetic acid, morpholinylethyl ester; and
[7-ethyl-3-methyl-8-oxamoyl-6-(2-(2-thienyl)benzyl)pyrrolo[1,2-a]pyrazin-1-yl]oxyacetic acid, sodium salt.

10. A pharmaceutical composition comprising of a sPLA₂ inhibitor useful for the treatment of renal dysfunction.

11. The use of a sPLA₂ inhibitor in combination with therapeutically effective agents and or procedures selected from the group consisting of dialysis treatment to remove harmful toxins; drugs to restore salt and water balance; for the delay, prevention and/or treatment of acute or chronic renal failure.

12. The use of a sPLA₂ inhibitor in combination with atrial naturetic factor (ANF) for the delay, prevention and/or treatment of acute and chronic renal failure in a mammal.

13. The use of a sPLA₂ in combination with erythropoetin to stimulate red cell production in a mammal.

14. The present invention is also the use of a sPLA₂ inhibitor in combination with OKT3™ to prevent kidney rejection or reduce the symptoms associated with administration of OKT3™.

15. A method as in any one of claims 1 or 2 or 3 or 4 or 5 or 6 or 7 or 8 or 9 or 10 wherein the administration of sPLA₂ inhibitor compound is in an amount of from 0.01 mg/kg/day to 100 mg/kg/day.

16. The method as in any one of claims 1 or 2 or 3 or 4 or 5 or 6 or 7 or 8 or 9 or 10 wherein the administration of sPLA₂ compound is oral.

17. The method as in any one of claims 1 or 2 or 3 or 4 or 5 or 6 or 7 or 8 or 9 or 10 wherein treatment is of an animal afflicted with renal dysfunction and the sPLA₂ inhibitor is administered in a therapeutically effective amount to achieve an animal blood level inhibitor concentration of from 10 to 3000 nanograms/ml.

18. The method as in any one of claims 1 or 2 or 3 or 4 or 5 or 6 or 7 or 8 or 9 or 10 wherein the therapeutically effective amount is in the form of a pharmaceutical formulation comprising the sPLA₂ inhibitor and a suitable carrier or excipient therefor.

19. The method as in claim 11 wherein the therapeutically effective amount is in the form of a pharmaceutical formulation comprising: a sPLA₂ inhibitor compound and a suitable carrier or excipient therefor.

20. Use of a sPLA₂ inhibitor selected from 1H-indole-3-glyoxylamide, 1H-indole-3-hydrazide, 1H-indole-3-acetamide, 1H-indole-1-glyoxylamide, 1H-indole-1-hydrazide, 1H-indole-1-acetamide, indolizine-1-acetamide, indolizine-1-acetic acid hydrazide, indolizine-1-glyoxylamide, indene-1-acetamides, indene-1-acetic acid hydrazide, indene-1-glyoxylamide, carbazoles, tetrahydrocarbazoles, pyrazoles, phenyl glyoxamides, pyrroles, naphthyl glyoxamides, naphthyl acetamide, and phenyl acetamide for the manufacture of a medicament for therapeutic treatment of renal dysfunction.

21. Use of a compound selected from compounds represented by one of one of the following formulae 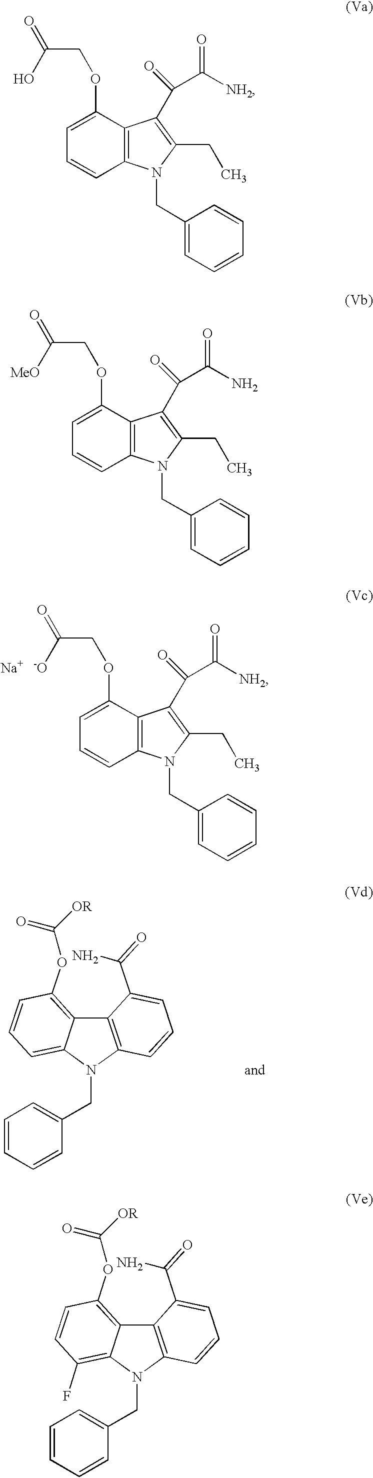
wherein R is independently selected from the group consisting of hydrogen, alkyl, aryl and heteroaryl for the manufacture of a medicament for therapeutic treatment of renal-dysfunction.

22. The method as in any one of claims 1 or 2 or 3 or 4 or 5 or 6 or 7 or 8 or 9 or 10 wherein the administration is transdermal.

23. The method as in any one of claims 1 or 2 or 3 or 4 or 5 or 6 or 7 or 8 or 9 or 10 wherein the administration is intramuscular.

24. Use of a composition including members selected from the group comprising: 1H-indole-3-glyoxylamide, 1H-indole-3-hydrazide, 1H-indole-3-acetamide, 1H-indole-1-glyoxylamide, 1H-indole-1-hydrazide, 1H-indole-1-acetamide, indolizine-1-acetamide, indolizine-1-acetic acid hydrazide, indolizine-1-glyoxylamide, indene-1-acetamide, indene-1-acetic acid hydrazide, indene-1-glyoxylamide, carbazole, tetrahydrocarbazole, pyrazole, phenyl glyoxamide, pyrrole, naphthyl glyoxamide, naphthyl acetamide, phenyl acetamide, 9H-carbazole, 9-benzylcarbazole, 1-(9H-benzylcarbazol-1-halo-4-yloxy-5-alkylamido) alkylacetate, 1-(9H-benzylcarbazol-4-yloxy-5-alkylamido) alkylacetate, 1-(9H-benzylcarbazol-1-halo-4-yloxy-5-alkylamido) acetic acid, 1-(9H-benzylcarbazol-4-yloxy-5-alkylamido) acetic acid and mixtures thereof for the manufacture of a medicament for the therapeutic treatment of renal dysfunction.
